(19) United States
(12) Patent Application Publication
(10) Pub. No.: US 2023/0332137 A1
(43) Pub. Date: Oct. 19, 2023

(54) METHODS AND COMPOSITIONS FOR NUCLEIC ACID ASSEMBLY

(71) Applicant: Mark S. CHEE, Encinitas, CA (US)

(72) Inventor: Mark S. CHEE, Encinitas, CA (US)

(21) Appl. No.: 18/026,096

(22) PCT Filed: Sep. 13, 2021
(86) PCT No.: PCT/US2021/050126

§ 371 Date: Mar. 13, 2023

Related U.S. Application Data
(60) Provisional application No. 63/078,178, filed on Sep. 14, 2020.

(51) Int. Cl. C12N 15/10

(52) U.S. Cl. CPC C12N 15/1065

(57) ABSTRACT

Disclosed in certain aspects herein are methods and compositions for the assembly of genes and even larger nucleic acid molecules, and methods of using assembled nucleic acids, e.g., as synthetic biology tools and/or products.

### CROSS-REFERENCE TO RELATED APPLICATIONS

This application claims priority from U.S. provisional application No. 63/078,178, filed Sep. 14, 2020, entitled “Methods and Compositions for Nucleic Acid Assembly,” the contents of which are incorporated by reference herein in their entirety for all purposes.

### FIELD

The present disclosure generally relates to methods and compositions for designing and/or generating a target nucleic acid.

### BACKGROUND

Advances in DNA sequencing techniques, e.g., next-generation sequencing, have allowed researchers to access the genetic codes of many organisms. While valuable insights have been made from reading DNA, synthetic biology researchers aim to further understand biological systems by synthesizing, or writing, DNA. An area of study with far-reaching applications, synthetic biology can facilitate the development of novel products (e.g., therapeutics), analytical tools, and manufacturing processes. Progress in the field critically depends on improved nucleic acid (e.g., gene) synthesis capabilities.

Methods of nucleic acid synthesis have evolved to address challenges related to cost, quantity, and sequence fidelity. This progress has enabled assembly of DNA constructs that encode bacterial genomes (Gibson et al. (2008) Science 319(5867): 1215-1220; and Hutchinson et al. (2016) Science 351(6280):aad6253) and eukaryotic chromosomes (Annaluru et al. (2014) Science 344(6179):55-58), far surpassing the length of a single gene. However, achievement of proof-of-concept large-scale DNA synthesis is not without technical challenges (Hughes and Ellington (2017) Cold Spring Harb Perspect Biol 9:a023812). For example, whereas the cost of sequencing has decreased precipitously over time, the cost of gene synthesis and oligonucleotide synthesis in general has not kept pace. The cost of gene synthesis is typically directly tied to the cost of oligonucleotide synthesis from which the genes are made, and the cost of oligonucleotide synthesis has not decreased appreciably in more than a decade, generally ranging from $0.05 to $0.15 per base depending on the synthesis scale, the length of the oligonucleotide, and the supplier. Special (i.e., higher) prices typically apply to sequences with “difficult” features and can raise the cost dramatically. Obstacles to low cost and high sequence fidelity synthetic DNA on the chromosome scale have yet to be overcome to truly enable the broad-ranging applications of synthetic biology. Compositions and methods to reduce the cost, increase the throughput, and ensure the fidelity of nucleic acid synthesis are needed, e.g., to close the DNA read-write cost gap. The present disclosure addresses these and other needs.

### BRIEF SUMMARY

The synthesis of artificial nucleic acids (e.g., synthetic DNA) is often referred to generically as “gene synthesis,” which comprises the synthesis of gene-length pieces of DNA (e.g., 250-2000 bp) directly from shorter single-stranded synthetic oligonucleotides. To enable larger-scale engineering efforts, longer nucleic acids, e.g., molecules of chromosome- or genome-lengths, may be needed.

Oligos for gene synthesis generally can be obtained from vendors as pools of hundreds to tens of thousands and potentially millions of oligos. However, the number of additions that is practical to carry out in a one-pot reaction is much less, which is generally limited by the specificity of joining reactions, oligo synthesis error rates, and/or joining error rates. Typically, the number of additions (e.g., joining oligos to form a longer oligo) in a one-pot reaction is on the order of tens. Therefore, there is a mismatch in scale of oligo synthesis and scale of the assembly reactions. In some aspects, the present disclosure describes an approach to bridge that gap in a scalable way. In some embodiments, hairpin oligos can be used in one-pot additive gene synthesis as well as other gene synthesis schemes.

In some embodiments, a hairpin oligo is designed to contain a capture tag sequence in a single-stranded loop region of the oligo, and a plurality of sets of oligos can be designed. For example, each set of oligos may be assembled in a one-pot reaction in parallel with other sets, and the oligos are sequentially added to a growing assembled product, e.g., in a predetermined order, in order to generate a target nucleic acid.

In some embodiments, each set of oligos intended to be combined in a one-pot reaction can be designed to have the same capture tag sequence or a set of capture tag sequences. For example, oligos may have a small set of capture tag sequences (e.g., two, three, four, five, or more capture tag sequences) that can be captured by a bead comprising one or more capture oligos capable of hybridizing to the set of capture tag sequences, thereby capturing the oligos of the same set on the bead. In this way, a large pool, in some embodiments millions of oligos, can be designed and partitioned into subsets, e.g., with one subset being immobilized on one bead. The partition, e.g., an emulsion droplet, can be used as a one-pot reaction volume for nucleic acid assembly, where reagents including the oligos and enzymes may be present in high concentrations for efficient reactions in the droplet. The oligo sequences, for example including the capture tag sequences, can be designed to enable more than one round of partitioning, if desired.

In some embodiments, a corresponding set of capture oligos is used to isolate each subset of “building block” oligos, such as a seed oligo, an addition oligo, and a terminal oligo, which can be the last addition oligo of a designed sequential addition process. In some embodiments, the capture oligos are attached to a support (e.g., bead or solid substrate), covalently or via a binding pair (e.g., biotin and streptavidin binding). For instance, a capture oligo may comprise a biotin moiety, and the oligos to be captured may comprise a biotin-binding moiety, such as an avidin or streptavidin or a variant, mutein, or fragment thereof. For instance, a capture oligo may comprise an avidin or streptavidin or a variant, mutein, or fragment thereof, and the oligos to be captured may comprise a avidin/streptavidin-binding moiety, such as a biotin or a variant, mutein, or fragment thereof. The capture oligos can comprise any suitable nucleic acids, such as natural nucleic acids (e.g., DNA or RNA), synthetic nucleic acids, modified nucleic acids, XNAs such as LNAs, HNAs, CeNAs, TNAs, GNAs, LNAs, PNAs, FANAs, or other nucleic acids or related polymers. In this way a pool of capture beads can be used to partition even a large number of different oligos in a simple, homogenous capture reaction.

In some embodiments, following capture and washing to remove non-specifically bound oligos, the beads can be partitioned into droplets in an emulsion. In some embodiments, each bead captures a mix of oligos that belong to the same subset by virtue of sharing a common capture tag sequence. In some embodiments, the emulsion comprises reagents for additive one-plot gene assembly and one or more starting sequences (e.g., a seed DNA oligo), which may be in solution, attached to the capture bead along with the capture oligos, or on a separate bead.

In some embodiments, the hairpin oligos are released from the capture oligos (e.g., by heating) and the addition reactions are carried out. In some embodiments, if necessary, the capture oligos on the bead can be prepared with blocked termini so that they cannot participate in the reactions such as the sequential addition of oligos.

In some embodiments, using bead capture and emulsion partitioning provides a number of advantages, including simplicity and scalability, and the ability to achieve high reagent concentrations inside the droplets to facilitate rapid reaction, by virtue of the small droplet volume. In some embodiments, methods other than bead capture and emulsion partitioning can be used. For instance, similar advantages can be achieved by appropriately designed microfluidics devices, which may also permit the handling and processing of beads.

In some embodiments, besides the synthesis enzymes (e.g., one or more ligases, one or more polymerases, and one or more restriction enzymes such as Type IIS enzymes), it is also possible to include primers such as PCR primers, so that an assembled product (e.g., a full length target nucleic acid to be produced or any intermediate thereof during assembly) can be amplified. In some embodiments, the primers comprise one or more universal primers, or one or more common primers to one or more subsets of the assembled products. In some embodiments, one or more ends of one or more assembled products are modified or processed, e.g., removed, prior to a next stage or a higher level of assembly. For example, sequences in one or more assembled products that contain a universal or common primer binding sequence may be removed to assemble an assembled product into an even longer product.

In some embodiments, the methods disclosed herein comprise assembling hairpin oligos, e.g., shorter oligos synthesized using variations of the phosphoramidite chemistry methods either on traditional column-based synthesizers or microarray-based synthesizers, which are typically commercially available at a reasonable price per base. These hairpin oligos are assembled in a first level of assembly in a highly parallel, multiplexed, and scalable way. In some embodiments, the methods disclosed herein further comprise a next tier of assembly, e.g., a second level, third level, or even higher level of assembly, where the assembled products from the previous level are further assembled into longer products. In some embodiments, the next tier or higher level assembly comprises the sequential addition reactions involving hairpin oligos as in the first level of assembly. In some embodiments, the methods disclosed herein comprise a first level of assembly and a second level of assembly, both levels involving sequential addition of hairpin oligos. In some embodiments, the methods disclosed herein comprise a first, a second, and a third level of assembly, all three levels involving sequential addition of hairpin oligos. In some embodiments, the methods disclosed herein comprise a first, a second, a third, and a fourth level of assembly, all four levels involving sequential addition of hairpin oligos.

Also provided herein are methods and compositions for identifying and/or selecting assembled molecules having one or more correct target sequences. In some embodiments, an assembled product comprises one or more unique molecular identifier (UMI) sequences, which may be used to identify products having the correct target sequences. In some embodiments, one or more primers that are complementary or capable of hybridizing to the one or more UMI sequences are used to amplify and/or select products having the correct target sequences. In some embodiments, one or more capture oligos (e.g., on a bead) that are complementary or capable of hybridizing to the one or more UMI sequences are used to capture and/or select products having the correct target sequences. In some embodiments, the one or more UMI sequences are complementary or capable of hybridizing to both the one or more primers and the one or more capture oligos.

In some embodiments, provided herein are methods of assembling a target polynucleotide, comprising partitioning a plurality of polynucleotides into a contained reaction volume, wherein the plurality of polynucleotides comprise a first polynucleotide and a second polynucleotide, wherein the second polynucleotide is attached to a support, the first polynucleotide comprises a first subsequence of a target polynucleotide, wherein the first polynucleotide comprises a single-stranded 3′ end sequence, the second polynucleotide comprises, in the 3′ to 5′ direction (i) a single-stranded 3′ end sequence, (ii) a second subsequence of the target polynucleotide, (iii) a Type IIS restriction enzyme recognition sequence, and (iv) a complementary sequence capable of hybridizing to all or a portion of the second subsequence, and the second polynucleotide is capable of forming a hairpin molecule comprising a 3′ overhang, a stem formed by intramolecular nucleotide base pairing between all or a portion of the second subsequence and the complementary sequence, and a loop comprising the Type IIS restriction enzyme recognition sequence in a configuration that is not cleaved by a Type IIS restriction enzyme; wherein the first polynucleotide and/or the second polynucleotide optionally further comprise a tag, a barcode, an amplification site, a unique molecular identifier (UMI), or any combination thereof; and wherein the first and second polynucleotides are connected within the contained reaction volume, thereby assembling the first and second subsequences. In some embodiments, the first and/or the second polynucleotide can further comprise a tag, a barcode, an amplification site, a unique molecular identifier (UMI), or any combination thereof.

In some embodiments, the first polynucleotide can comprise two nucleic acid strands forming a duplex. In any of the preceding embodiments, the first polynucleotide can be capable of forming one or more hairpins. In any of the preceding embodiments, the first polynucleotide and/or the last polynucleotide (e.g., a terminal oligo) can comprise one or more barcodes and/or one or more tags, e.g., a capture tag sequence. In any of the preceding embodiments, the first polynucleotide can comprise a capture tag sequence.

In any of the preceding embodiments, a useful sequence, such as the one or more barcodes and/or one or more tags, can be part of the target sequence that is assembled. The useful sequence may include any one or more of an adapter sequence (e.g., a universal adapter sequence or a sequencing adapter, e.g., P5 and/or P7), a tag sequence (e.g., for hybridization to one or more capture oligos on a support), a priming site (e.g., a universal primer binding sequence, e.g., for amplification of an assembled product), a cleavage site or sequence (e.g., a restriction enzyme recognition sequence and cleavage site), a unique molecular identifier (UMI), a unique identifier (UID), and a barcode, any one or more of which may be unique to a target sequence or to a subset of target sequences among a plurality of target sequences.

For example, a capture tag sequence can span the junction of two subsequences that are correctly assembled, and a capture oligo complementary to the capture tag sequence may be used to capture and/or enrich the correctly assembled sequence. In some embodiments, a capture tag sequence useful for identifying and/or selecting a correctly assembled sequence is not present in any of the individual subsequences or building block oligos comprising the subsequences. In some embodiments, a capture oligo complementary to the capture tag sequence does not capture and/or enrich any of the individual subsequences or building block oligos comprising the subsequences.

In any of the preceding embodiments, prior to connecting the first and second polynucleotides, the first polynucleotide can be not attached to the support.

In any of the preceding embodiments, prior to connecting the first and second polynucleotides, the first polynucleotide can be attached to the support. In some embodiments, the first polynucleotide can be directly or indirectly attached to the support. In any of the preceding embodiments, the first polynucleotide can be covalently or noncovalently attached to the support or a linker, e.g., a cleavable linker attached to the support. In any of the preceding embodiments, the first polynucleotide can be attached to the support via hybridization (e.g., between a capture probe sequence directly or indirectly on the support and a capture tag sequence of the first polynucleotide), the interaction between a binding pair (e.g., biotin/streptavidin binding), a covalent bond, or any combination thereof.

In any of the preceding embodiments, the first polynucleotide can remain attached to the support during and/or after connecting the first and second polynucleotides. In any of the preceding embodiments, the first polynucleotide can be released from the support after the first and second polynucleotides are connected.

In any of the preceding embodiments, the first polynucleotide can be released from the support before the first and second polynucleotides are connected.

In any of the preceding embodiments, the releasing can comprise heating the contained reaction volume and/or enzymatic cleavage of the first polynucleotide or a cleavable linker between the first polynucleotide and a support.

In any of the preceding embodiments, the second polynucleotide can comprise one or more barcodes and/or one or more tags, e.g., a capture tag sequence. In any of the preceding embodiments, the second polynucleotide can comprise a capture tag sequence.

In any of the preceding embodiments, the second polynucleotide can be directly or indirectly attached to the support. In any of the preceding embodiments, the second polynucleotide can be covalently or noncovalently attached to the support or a linker, e.g., a cleavable linker attached to the support. In any of the preceding embodiments, the second polynucleotide can be attached to the support via hybridization (e.g., between a capture probe sequence directly or indirectly on the support and a capture tag sequence of the second polynucleotide), the interaction between a binding pair (e.g., biotin/streptavidin binding), a covalent bond, or any combination thereof.

In any of the preceding embodiments, prior to connecting the first and second polynucleotides, the second polynucleotide can be not released from the support. In some embodiments, the second polynucleotide can remain attached to the support during and/or after connecting the first and second polynucleotides. In any of the preceding embodiments, the second polynucleotide can be released from the support after the first and second polynucleotides are connected.

In any of the preceding embodiments, prior to connecting the first and second polynucleotides, the second polynucleotide can be released from the support.

In any of the preceding embodiments, the releasing can comprise heating the contained reaction volume and/or enzymatic cleavage of the second polynucleotide or a cleavable linker between the second polynucleotide and a support.

In any of the preceding embodiments, the first and second polynucleotides can be connected in the contained reaction volume when both are not attached to the support.

In any of the preceding embodiments, the second polynucleotide can form the hairpin molecule before and/or during connecting the first and second polynucleotides.

In any of the preceding embodiments, the 5′ end of the second polynucleotide can be blocked from ligation, extension, and/or hybridization. In any of the preceding embodiments, the 5′ end of the second polynucleotide can be blocked from ligation. For instance, the 5′ end of the second polynucleotide may lack a 5′ phosphate group and/or may comprise a blocking modification or group.

In any of the preceding embodiments, the second polynucleotide can further comprise, between the second subsequence and the complementary sequence, a sequence comprising one or more barcodes and/or one or more tags, e.g., a capture tag sequence. In some embodiments, the sequence comprising one or more barcodes and/or one or more tags can be between the Type IIS restriction enzyme recognition sequence and the complementary sequence.

In any of the preceding embodiments, the second polynucleotide can further comprise a 5′ end sequence that does not hybridize to the single-stranded 3′ end sequence or the second subsequence. In some embodiments, the 5′ end sequence can comprise one or more barcodes and/or one or more tags, e.g., a capture tag sequence. In any of the preceding embodiments, the 5′ end sequence can be blocked from ligation, extension, and/or hybridization. In any of the preceding embodiments, the 5′ end sequence can be blocked from ligation.

In any of the preceding embodiments, the stem can comprise one or more bulged bases in either one or both strands of the stem. In some embodiments, the stem can comprise a bulge sequence in the strand comprising the complementary sequence. In any of the preceding embodiments, the bulge sequence can be capable of forming one or more internal hairpins. In any of the preceding embodiments, the bulge sequence can comprise one or more barcodes and/or one or more tags, e.g., a capture tag sequence. In any of the preceding embodiments, the stem can comprise a bulge sequence in the strand comprising the second subsequence.

In any of the preceding embodiments, the second subsequence can be capable of forming one or more hairpins internal to the hairpin molecule formed by the second polynucleotide.

In any of the preceding embodiments, the second polynucleotide can further comprise an intervening sequence between the second subsequence and the Type IIS restriction enzyme recognition sequence. In some embodiments, the intervening sequence can be capable of being cleaved from the second subsequence by the Type IIS restriction enzyme when the second polynucleotide forms a duplex with a complementary strand.

In any of the preceding embodiments, there can be no intervening sequence between the second subsequence and the Type IIS restriction enzyme recognition sequence.

In any of the preceding embodiments, the 3′ end of the 3′ overhang can be not blocked from ligation, extension, and/or hybridization.

In any of the preceding embodiments, the 3′ overhang can be between about 1 and about 100 nucleotides in length. In any of the preceding embodiments, the 3′ overhang can be between about 2 and about 20 nucleotides in length. In any of the preceding embodiments, the 3′ overhang can be between about 2 and about 15 nucleotides in length, e.g., 2, 3, 4, 5, 6, 7, 8, 9, or 10 nucleotides in length.

In any of the preceding embodiments, the contained reaction volume can be an emulsion droplet. In any of the preceding embodiments, the contained reaction volume can comprise one or more Type IIS restriction enzymes. In any of the preceding embodiments, the contained reaction volume can comprise one or more polymerases. In any of the preceding embodiments, the contained reaction volume can comprise one or more ligases. In any of the preceding embodiments, the contained reaction volume can comprise one or more nucleases other than a Type IIS restriction enzyme, e.g., one or more exonucleases and/or one or more endonucleases. In any of the preceding embodiments, the contained reaction volume can comprise one or more exonucleases and/or one or more endonucleases.

In any of the preceding embodiments, the second polynucleotide can form the hairpin molecule, and all or a portion of the 3′ overhang can hybridize to all or a portion of the single-stranded 3′ end sequence of the first subsequence to form a hybridization complex. In some embodiments, the hybridization complex can comprise (i) a nick or gap between the 3′ end of the first polynucleotide and the 5′ end of the second polynucleotide, and (ii) a nick or gap between the 5′ end of the first polynucleotide and the 3′ end of the second polynucleotide.

In any of the preceding embodiments, a polymerase can be capable of extending the 3′ end sequence of the first subsequence in the hybridization complex using the second polynucleotide as template.

In any of the preceding embodiments, a polymerase can be incapable of extending the 3′ end sequence of the first subsequence in the hybridization complex using the second polynucleotide as template, e.g., when the hybridization complex comprises two nicks, one on each strand, that are between about 1 and about 10 nucleotides apart, e.g., between about 1 and about 6 nucleotides apart. In some embodiments, the nick or gap between the 5′ end of the first polynucleotide and the 3′ end of the second polynucleotide can be filled, e.g., by ligation of the nick, or by hybridization of a filler sequence to fill in the gap followed by ligation of the filler sequence. In any of the preceding embodiments, the nick between the 5′ end of the first polynucleotide and the 3′ end of the second polynucleotide can be ligated by a ligase, whereas the nick between the 3′ end of the first polynucleotide and the 5′ end of the second polynucleotide can be not ligated by the ligase, e.g., wherein the 5′ end of the second polynucleotide is blocked from ligation, e.g., wherein the 5′ end nucleotide of the second polynucleotide is dephosphorylated.

In any of the preceding embodiments, a double-stranded polynucleotide comprising the first subsequence, the second subsequence, the Type IIS restriction enzyme recognition sequence, and optionally the complementary sequence, can be generated by a polymerase that extends the 3′ end sequence of the first subsequence using the second polynucleotide as template. In some embodiments, a Type IIS restriction enzyme can recognize the Type IIS restriction enzyme recognition sequence and cleave the double-stranded polynucleotide, thereby generating a cleaved double-stranded polynucleotide that can comprise the first subsequence connected to the second subsequence. In some embodiments, the cleaved double-stranded polynucleotide can comprise a single-stranded 3′ end sequence. In some embodiments, the single-stranded 3′ end sequence of the cleaved double-stranded polynucleotide can be between about 2 and about 10 nucleotides in length.

In any of the preceding embodiments, the plurality of polynucleotides can further comprise a third polynucleotide.

In some embodiments, the third polynucleotides can be attached to the support and can comprise, in the 3′ to 5′ direction (i) a single-stranded 3′ end sequence, (ii) a third subsequence of the target polynucleotide, (iii) a Type IIS restriction enzyme recognition sequence, and (iv) a complementary sequence capable of hybridizing to all or a portion of the third subsequence wherein the third polynucleotide can be capable of forming a hairpin molecule comprising a 3′ overhang, a stem formed by intramolecular nucleotide base pairing between all or a portion of the third subsequence and the complementary sequence, and a loop comprising the Type IIS restriction enzyme recognition sequence in a configuration that can be not cleaved by a Type IIS restriction enzyme, and wherein the first, second, and third polynucleotides can be connected sequentially within the contained reaction volume, thereby assembling the first, second, and third subsequences.

In any of the preceding embodiments, the support can comprise a particle, a bead, a solid substrate, a plate, a well, an array, a membrane, or a combination thereof. In any of the preceding embodiments, the support can comprise a bead, such as a magnetic bead or a dissolvable or disruptable bead such as gel beads disclosed in U.S. Pat. No. 10,876,147 incorporated herein by reference in its entirety for all purposes.

In any of the preceding embodiments, the target polynucleotide can be at least about 100, about 250, about 500, about 1,000, about 2,500, about 5,000, about 10,000, about 25,000, or about 50,000 nucleotides in length.

In any of the preceding embodiments, the plurality of polynucleotides can comprise 3, 4, 5, 6, 7, 8, 9, 10 or more polynucleotides each comprising a subsequence of the target polynucleotide.

In any of the preceding embodiments, the target polynucleotide can be a DNA molecule, and the target polynucleotide can optionally comprise a gene or fragment thereof, a gene cluster, a mitochondrial DNA or fragment thereof, a chromosome or fragment thereof, or a genome. In any of the preceding embodiments, the target polynucleotide can comprise a gene or fragment thereof, a gene cluster, a mitochondrial DNA or fragment thereof, a chromosome or fragment thereof, or a genome.

In any of the preceding embodiments, the first polynucleotide and/or the second polynucleotide can further comprise a capture tag sequence, an amplification site, and a UMI, wherein the UMI sequence can be complementary to the capture tag sequence and/or the amplification site.

Also provided herein are methods of assembling a plurality of target polynucleotides, comprising (a) for each target polynucleotide, partitioning a plurality of polynucleotides into a contained reaction volume, wherein the plurality of polynucleotides comprise a first polynucleotide and a second polynucleotide, wherein the second polynucleotide is attached to a support, the first polynucleotide comprises a first subsequence of the target polynucleotide, wherein the first polynucleotide comprises a single-stranded 3′ end sequence, the second polynucleotide comprises, in the 3′ to 5′ direction (i) a single-stranded 3′ end sequence, (ii) a second subsequence of the target polynucleotide, (iii) a Type IIS restriction enzyme recognition sequence, and (iv) a complementary sequence capable of hybridizing to all or a portion of the second subsequence, and the second polynucleotide is capable of forming a hairpin molecule comprising a 3′ overhang, a stem formed by intramolecular nucleotide base pairing between all or a portion of the second subsequence and the complementary sequence, and a loop comprising the Type IIS restriction enzyme recognition sequence in a configuration that is not cleaved by a Type IIS restriction enzyme; and (b) within each contained reaction volume, connecting the first and second polynucleotides, thereby assembling the first and second subsequences, wherein the assembly of subsequences of each target polynucleotide is carried out in parallel.

In some embodiments, the methods can further comprise designing and/or obtaining the plurality of polynucleotides for each target polynucleotide. In some embodiments, the methods can further comprise designing the plurality of polynucleotides for each target polynucleotide.

In any of the preceding embodiments, the subsequences in the plurality of polynucleotides for each target polynucleotide can be between about 20 and about 200 nucleotides in length.

In any of the preceding embodiments, the plurality of polynucleotides for each target polynucleotide can be synthesized, and the synthesis can comprise base-by-base synthesis.

In any of the preceding embodiments, the partitioning can comprise enriching polynucleotides comprising subsequences of a given target polynucleotide, but not polynucleotides comprising subsequences of other target polynucleotides, in the contained reaction volume.

In any of the preceding embodiments, the partitioning can comprise capturing all or a subset of the plurality of polynucleotides for each target polynucleotide on a bead that can be specific for the target polynucleotide. In some embodiments, the bead can comprise a capture probe that specifically binds to a capture tag that can be unique for the target polynucleotide, wherein the capture tag can be common in all or a subset of the plurality of polynucleotides comprising subsequences of the target polynucleotide. In any of the preceding embodiments, the partitioning can comprise encapsulating the bead in an emulsion droplet, thereby generating a plurality of emulsion droplets for parallel assembly of the plurality of target polynucleotides. In some embodiments, the methods can further comprise releasing all or a subset of the polynucleotides captured on the beads into the emulsion droplets. In any of the preceding embodiments, the parallel assembly of the plurality of target polynucleotides can be carried out in each emulsion droplet by one or more concerted reaction cycles. In some embodiments, the one or more concerted reaction cycles can comprise an isothermal reaction. In any of the preceding embodiments, the one or more concerted reaction cycles can comprise sequential reactions of hybridization, ligation by a ligase, primer extension by a polymerase, and cleavage by a Type IIS restriction enzyme.

In any of the preceding embodiments, the assembly of all or a subset of the plurality of target polynucleotides can be unidirectional.

In any of the preceding embodiments, the assembly of all or a subset of the plurality of target polynucleotides can be bidirectional.

Also provided herein are methods of assembling a target polynucleotide, comprising (a) partitioning a plurality of polynucleotides into an emulsion droplet, wherein the plurality of polynucleotides comprise: (i) a first polynucleotide optionally attached to a bead, and (ii) a second polynucleotide attached to the bead, the first polynucleotide comprises a first subsequence of a target polynucleotide, wherein the first polynucleotide comprises a single-stranded 3′ end sequence, the second polynucleotide comprises, in the 3′ to 5′ direction (i) a single-stranded 3′ end sequence capable of hybridizing to the single-stranded 3′ end sequence of the first polynucleotide, (ii) a second subsequence of the target polynucleotide, (iii) a Type IIS restriction enzyme recognition sequence, and (iv) a complementary sequence capable of hybridizing to all or a portion of the second subsequence, and the second polynucleotide further comprises a tag sequence and/or a barcode sequence 5′ to the Type IIS restriction enzyme recognition sequence; (b) in the emulsion droplet, releasing the second polynucleotide from the bead, wherein the second polynucleotide forms a hairpin molecule comprising a 3′ overhang, a stem formed by intramolecular nucleotide base pairing between all or a portion of the second subsequence and the complementary sequence, and a loop comprising the Type IIS restriction enzyme recognition sequence in a configuration that is not cleaved by a Type IIS restriction enzyme; (c) allowing the 3′ overhang of the hairpin molecule to hybridize to the single-stranded 3′ end sequence of the first polynucleotide, wherein the 5′ end of the hairpin molecule is optionally blocked from ligation to the 3′ end of the first polynucleotide after hybridization; (d) optionally ligating the 3′ end of the hairpin molecule to the 5′ end of the first polynucleotide; (e) extending the 3′ end sequence of the first polynucleotide using the second polynucleotide as template, thereby generating a double-stranded polynucleotide comprising the first subsequence, the second subsequence, the Type IIS restriction enzyme recognition sequence, and optionally the complementary sequence, the tag sequence, and/or the barcode sequence; and (f) cleaving the double-stranded polynucleotide using a Type IIS restriction enzyme, thereby generating a cleaved double-stranded polynucleotide comprising the first subsequence and the second subsequence, wherein the cleaved double-stranded polynucleotide comprises a single-stranded 3′ end sequence, and optionally wherein the single-stranded 3′ end sequence is between about 2 and about 10 nucleotides in length, thereby assembling the first and second subsequences. In some embodiments, the 5′ end of the hairpin molecule can be blocked from ligation to the 3′ end of the first polynucleotide after hybridization. In some embodiments, the method can further comprise (d) ligating the 3′ end of the hairpin molecule to the 5′ end of the first polynucleotide.

In some embodiments, the first polynucleotide can be attached to the bead prior to the partitioning step. The bead can be any suitable bead such as a magnetic bead or a dissolvable or disruptable bead such as gel beads.

In some embodiments, the partitioning step can comprise attaching the first polynucleotide and the second polynucleotide to the bead, and the releasing step optionally can comprise releasing the first polynucleotide from the bead. In some embodiments, the releasing step can comprise releasing the first polynucleotide from the bead.

In any of the preceding embodiments, the first polynucleotide and/or the second polynucleotide can be directly or indirectly attached to the bead. In any of the preceding embodiments, the first polynucleotide and/or the second polynucleotide can be covalently or noncovalently attached to the bead or a linker, e.g., a cleavable linker between the polynucleotide(s) and the bead. In any of the preceding embodiments, the first polynucleotide and/or the second polynucleotide can be attached to the bead via hybridization (e.g., between a capture probe sequence directly or indirectly on the bead and a capture tag sequence of the first polynucleotide and/or the second polynucleotide), the interaction between a binding pair (e.g., biotin/streptavidin binding), a covalent bond, or any combination thereof.

In any of the preceding embodiments, using a cleavable linker allows release of one or more polynucleotides (e.g., the first polynucleotide and/or the second polynucleotide) or assembled targets from the support, e.g., beads such as a magnetic bead or a dissolvable or disruptable bead such as gel beads. In some embodiments, the linker attachment is covalent so that it is not prone to dissociation, but can be cleaved later at an appropriate step.

In some embodiments, the first polynucleotide can be not attached to the bead prior to, during, or after the partitioning step. In some embodiments, the first polynucleotide can be provided in a reaction volume that can be partitioned to form the emulsion droplet. In some embodiments, the reaction volume can further comprise a ligase, a polymerase, a Type IIS restriction enzyme, and/or a nuclease other than a Type IIS restriction enzyme.

In any of the preceding embodiments, the first polynucleotide can comprise a hairpin. In some embodiments, the first polynucleotide can comprise a stem comprising all or a portion of the first subsequence and a loop comprising a tag sequence and/or a barcode sequence.

In any of the preceding embodiments, in the partitioning step, the plurality of polynucleotides can further comprise (iii) a third polynucleotide attached to the bead, the third polynucleotide can comprise, in the 3′ to 5′ direction (i) a single-stranded 3′ end sequence capable of hybridizing to the single-stranded 3′ end sequence of the cleaved double-stranded polynucleotide, (ii) a third subsequence of the target polynucleotide, (iii) a Type IIS restriction enzyme recognition sequence, and (iv) a complementary sequence capable of hybridizing to all or a portion of the third subsequence, and the third polynucleotide can further comprise a tag sequence and/or a barcode sequence 5′ to the Type IIS restriction enzyme recognition sequence. In some embodiments, the releasing step can further comprise releasing the third polynucleotide from the bead, wherein the third polynucleotide can form a hairpin molecule comprising a 3′ overhang, a stem formed by intramolecular nucleotide base pairing between all or a portion of the third subsequence and the complementary sequence, and a loop comprising the Type IIS restriction enzyme recognition sequence in a configuration that can be not cleaved by a Type IIS restriction enzyme. In some embodiments, the methods can further comprise (g) hybridizing the 3′ overhang of the hairpin molecule formed by the third polynucleotide to the single-stranded 3′ end sequence of the cleaved double-stranded polynucleotide, wherein the 5′ end of the hairpin molecule formed by the third polynucleotide can be blocked from ligation to the 3′ end of the first polynucleotide after hybridization. In some embodiments, the methods can further comprise (h) ligating the 3′ end of the hairpin molecule formed by the third polynucleotide to the 5′ end of the cleaved double-stranded polynucleotide. In some embodiments, the methods can further comprise (i) extending the 3′ end sequence of the cleaved double-stranded polynucleotide using the third polynucleotide as template, thereby generating a double-stranded polynucleotide comprising the first subsequence, the second subsequence, the third subsequence, the Type IIS restriction enzyme recognition sequence of the third polynucleotide, and optionally the complementary sequence, the tag sequence, and/or the barcode sequence of the third polynucleotide. In some embodiments, the methods can further comprise (j) cleaving the double-stranded polynucleotide using a Type IIS restriction enzyme, thereby generating a cleaved double-stranded polynucleotide comprising the first subsequence, the second subsequence, and the third subsequence, wherein the cleaved double-stranded polynucleotide can comprise a single-stranded 3′ end sequence, and optionally wherein the single-stranded 3′ end sequence can be between about 2 and about 10 nucleotides in length, hereby assembling the first, second, and third subsequences.

In any of the preceding embodiments, in the partitioning step, the plurality of polynucleotides can further comprise an n^{th} polynucleotide attached to the bead, wherein n can be an integer of 4 or greater, the n^{th} polynucleotide can comprise, in the 3′ to 5′ direction (i) a single-stranded 3′ end sequence capable of hybridizing to the single-stranded 3′ end sequence of a cleaved double-stranded polynucleotide comprising the first, second, . . . , and the (n−1)^{th} subsequences of the target polynucleotide, (ii) an n^{th} subsequence of the target polynucleotide, (iii) a Type IIS restriction enzyme recognition sequence, and (iv) a complementary sequence capable of hybridizing to all or a portion of the n^{th} subsequence, and the n^{th} polynucleotide can further comprise a tag sequence and/or a barcode sequence 5′ to the Type IIS restriction enzyme recognition sequence. In some embodiments, the releasing step can further comprise releasing the n^{th} polynucleotide from the bead, wherein the n^{th} polynucleotide can form a hairpin molecule comprising a 3′ overhang, a stem formed by intramolecular nucleotide base pairing between all or a portion of the n^{th} subsequence and the complementary sequence, and a loop comprising the Type IIS restriction enzyme recognition sequence in a configuration that can be not cleaved by a Type IIS restriction enzyme. In some embodiments, the methods can further comprise repeating a concerted reaction cycle comprising sequential reactions of hybridization, ligation by a ligase, primer extension by a polymerase, and cleavage by a Type IIS restriction enzyme, thereby assembling the first, second, . . . , and the (n−1)^{th} subsequences.

Also provided herein are methods of assembling a target polynucleotide, comprising (a) partitioning a plurality of polynucleotides into an emulsion droplet, wherein the plurality of polynucleotides comprise: (i) a first polynucleotide optionally attached to a bead, (ii) a second polynucleotide attached to the bead, and (iii) a third polynucleotide attached to the bead, the first polynucleotide comprises a first subsequence of a target polynucleotide and is double-stranded, comprising a single-stranded 3′ end sequence in the top strand and a single-stranded 3′ end sequence in the bottom strand, the second polynucleotide comprises, in the 3′ to 5′ direction (i) a single-stranded 3′ end sequence capable of hybridizing to the top strand single-stranded 3′ end sequence of the first polynucleotide, (ii) a second subsequence of the target polynucleotide, (iii) a Type IIS restriction enzyme recognition sequence, and (iv) a complementary sequence capable of hybridizing to all or a portion of the second subsequence, the second polynucleotide optionally further comprises a tag sequence and/or a barcode sequence 5′ to the Type IIS restriction enzyme recognition sequence, the third polynucleotide comprises, in the 3′ to 5′ direction (i) a single-stranded 3′ end sequence capable of hybridizing to the bottom strand single-stranded 3′ end sequence of the first polynucleotide, (ii) a third subsequence of the target polynucleotide, (iii) a Type IIS restriction enzyme recognition sequence, and (iv) a complementary sequence capable of hybridizing to all or a portion of the third subsequence, the third polynucleotide optionally further comprises a tag sequence and/or a barcode sequence 5′ to the Type IIS restriction enzyme recognition sequence; (b) in the emulsion droplet, releasing the second and third polynucleotides, and optionally the first polynucleotide, from the bead, wherein the second polynucleotide forms a hairpin molecule comprising a 3′ overhang, a stem formed by intramolecular nucleotide base pairing between all or a portion of the second subsequence and the complementary sequence, and a loop comprising the Type IIS restriction enzyme recognition sequence in a configuration that is not cleaved by a Type IIS restriction enzyme, and the third polynucleotide forms a hairpin molecule comprising a 3′ overhang, a stem formed by intramolecular nucleotide base pairing between all or a portion of the third subsequence and the complementary sequence, and a loop comprising the Type IIS restriction enzyme recognition sequence in a configuration that is not cleaved by a Type IIS restriction enzyme; (c) allowing the 3′ overhangs of the hairpin molecules formed by the second and third polynucleotides to hybridize to the top strand single-stranded 3′ end sequence and the bottom strand single-stranded 3′ end sequence, respectively, of the first polynucleotide, wherein the 5′ ends of the hairpin molecules are blocked from ligation to the 3′ ends of the first polynucleotide after hybridization; (d) ligating the 3′ ends of the hairpin molecules to the 5′ ends of the first polynucleotide; (e) extending the 3′ end sequences of the first polynucleotide using the second and third polynucleotides as template, thereby generating a double-stranded polynucleotide comprising the first subsequence flanked by the second subsequence on one side and the third subsequence on the other side, the Type IIS restriction enzyme recognition sequences, and optionally the complementary sequences, the tag sequence(s), and/or the barcode sequence(s); and (f) cleaving the double-stranded polynucleotide using a Type IIS restriction enzyme, thereby generating a cleaved double-stranded polynucleotide comprising the first subsequence flanked by the second subsequence on one side and the third subsequence on the other side, wherein the cleaved double-stranded polynucleotide comprises a single-stranded 3′ end sequence in the top strand and a single-stranded 3′ end sequence in the bottom strand, and optionally wherein the single-stranded 3′ end sequences are between about 2 and about 10 nucleotides in length, thereby assembling the first, second, and third subsequences.

In some embodiments, in the partitioning step, the plurality of polynucleotides can further comprise a fourth polynucleotide attached to the bead, and the fourth polynucleotide can comprise, in the 3′ to 5′ direction (i) a single-stranded 3′ end sequence capable of hybridizing to the top strand single-stranded 3′ end sequence of the cleaved double-stranded polynucleotide, (ii) a fourth subsequence of the target polynucleotide, (iii) a Type IIS restriction enzyme recognition sequence, and (iv) a complementary sequence capable of hybridizing to all or a portion of the fourth subsequence, and the fourth polynucleotide can optionally further comprise a tag sequence and/or a barcode sequence 5′ to the Type IIS restriction enzyme recognition sequence. In some embodiments, in the partitioning step, the plurality of polynucleotides can further comprise a fifth polynucleotide attached to the bead, and the fifth polynucleotide can comprise, in the 3′ to 5′ direction (i) a single-stranded 3′ end sequence capable of hybridizing to the bottom strand single-stranded 3′ end sequence of the cleaved double-stranded polynucleotide, (ii) a fifth subsequence of the target polynucleotide, (iii) a Type IIS restriction enzyme recognition sequence, and (iv) a complementary sequence capable of hybridizing to all or a portion of the fifth subsequence, and the fifth polynucleotide can optionally further comprise a tag sequence and/or a barcode sequence 5′ to the Type IIS restriction enzyme recognition sequence. In some embodiments, the releasing step further can comprise releasing the fourth and fifth polynucleotides from the bead, wherein the fourth polynucleotide can form a hairpin molecule comprising a 3′ overhang, a stem formed by intramolecular nucleotide base pairing between all or a portion of the fourth subsequence and the complementary sequence, and a loop comprising the Type IIS restriction enzyme recognition sequence in a configuration that can be not cleaved by a Type IIS restriction enzyme, and the fifth polynucleotide can form a hairpin molecule comprising a 3′ overhang, a stem formed by intramolecular nucleotide base pairing between all or a portion of the fifth subsequence and the complementary sequence, and a loop comprising the Type IIS restriction enzyme recognition sequence in a configuration that can be not cleaved by a Type IIS restriction enzyme.

In some embodiments, the methods can further comprise (g) hybridizing the 3′ overhangs of the hairpin molecules formed by the fourth and fifth polynucleotides to the top strand single-stranded 3′ end sequence and the bottom strand single-stranded 3′ end sequence, respectively, of the cleaved double-stranded polynucleotide, wherein the 5′ ends of the hairpin molecules can be blocked from ligation to the 3′ ends of the cleaved double-stranded polynucleotide after hybridization. In some embodiments, the methods can further comprise (h) ligating the 3′ ends of the hairpin molecules formed by the fourth and fifth polynucleotides to the 5′ ends of the cleaved double-stranded polynucleotide. In some embodiments, the methods can further comprise (i) extending the 3′ end sequences of the cleaved double-stranded polynucleotide using the fourth and fifth polynucleotides as template, thereby generating a double-stranded polynucleotide comprising: the first subsequence flanked by the second subsequence on one side and the third subsequence on the other side, which can be in turn flanked by the fourth subsequence and the fifth subsequence, respectively; the Type IIS restriction enzyme recognition sequences of the fourth and fifth polynucleotides; and optionally the complementary sequences, the tag sequence(s), and/or the barcode sequence(s) of the fourth and fifth polynucleotides. In some embodiments, the methods can further comprise (j) cleaving the double-stranded polynucleotide using a Type IIS restriction enzyme, thereby generating a cleaved double-stranded polynucleotide comprising the first subsequence flanked by the second subsequence on one side and the third subsequence on the other side, which can be in turn flanked by the fourth subsequence and the fifth subsequence, respectively, wherein the cleaved double-stranded polynucleotide can comprise a single-stranded 3′ end sequence in the top strand and a single-stranded 3′ end sequence in the bottom strand, and optionally wherein the single-stranded 3′ end sequences can be between about 2 and about 10 nucleotides in length, thereby assembling the first, second, third, fourth, and fifth subsequences.

Also provided herein are methods of assembling a target polynucleotide, comprising (a) partitioning a plurality of polynucleotides into an emulsion droplet, wherein the plurality of polynucleotides comprise: (i) a first polynucleotide optionally attached to a bead, and (ii) a second polynucleotide attached to the bead, the first polynucleotide comprises a first subsequence of a target polynucleotide, wherein the first polynucleotide comprises a single-stranded 3′ end sequence, the second polynucleotide comprises, in the 3′ to 5′ direction (i) a single-stranded 3′ end sequence capable of hybridizing to the single-stranded 3′ end sequence of the first polynucleotide, (ii) a second subsequence of the target polynucleotide, (iii) a Type IIS restriction enzyme recognition sequence, and (iv) a complementary sequence capable of hybridizing to all or a portion of the second subsequence, and the second polynucleotide further comprises a tag sequence and/or a barcode sequence 5′ to the Type IIS restriction enzyme recognition sequence; (b) in the emulsion droplet, releasing the second polynucleotide from the bead, wherein the second polynucleotide forms a hairpin molecule comprising a 3′ overhang, a stem formed by intramolecular nucleotide base pairing between all or a portion of the second subsequence and the complementary sequence, and a loop comprising the Type IIS restriction enzyme recognition sequence in a configuration that is not cleaved by a Type IIS restriction enzyme; (c) allowing the 3′ overhang of the hairpin molecule to hybridize to the single-stranded 3′ end sequence of the first polynucleotide to form a hybridization complex, wherein the 5′ end of the hairpin molecule is blocked from ligation to the 3′ end of the first polynucleotide after hybridization, and the hybridization complex comprises (i) a nick or gap between the 3′ end of the first polynucleotide and the 5′ end of the second polynucleotide, and (ii) a nick or gap between the 5′ end of the first polynucleotide and the 3′ end of the second polynucleotide, optionally wherein the nicks and gaps are more than about 6-10 nucleotides apart; (d) extending the 3′ end sequence of the first polynucleotide using the second polynucleotide as template, thereby generating a double-stranded polynucleotide comprising the first subsequence, the second subsequence, the Type IIS restriction enzyme recognition sequence, and optionally the complementary sequence, the tag sequence, and/or the barcode sequence; and (e) cleaving the double-stranded polynucleotide using a Type IIS restriction enzyme, thereby generating a cleaved double-stranded polynucleotide comprising the first subsequence and the second subsequence, wherein the cleaved double-stranded polynucleotide comprises a single-stranded 3′ end sequence, and optionally wherein the single-stranded 3′ end sequence is between about 2 and about 10 nucleotides in length, thereby assembling the first and second subsequences.

In some embodiments, the emulsion droplet can comprise a ligase, a polymerase, and a Type IIS restriction enzyme, and/or optionally a nuclease other than a Type IIS restriction enzyme.

In any of the preceding embodiments, the first polynucleotide can be attached to the support, e.g., to the bead such as a magnetic bead or a dissolvable or disruptable bead such as a gel bead. In any of the preceding embodiments, the second polynucleotide can be attached to the support, e.g., to the bead. In any of the preceding embodiments, the third polynucleotide can be attached to the support, e.g., to the bead. In any of the preceding embodiments, the fourth polynucleotide can be attached to the support, e.g., to the bead. In any of the preceding embodiments, the fifth polynucleotide can be attached to the support, e.g., to the bead.

In any of the preceding embodiments, the first polynucleotide can comprise a capture tag sequence. In any of the preceding embodiments, the second polynucleotide can comprise a capture tag sequence. In any of the preceding embodiments, the third polynucleotide can comprise a capture tag sequence. In any of the preceding embodiments, the fourth polynucleotide can comprise a capture tag sequence. In any of the preceding embodiments, the fifth polynucleotide can comprise a capture tag sequence.

In any of the preceding embodiments, the single-stranded 3′ end sequence is between about 2 and about 10 nucleotides in length.

Also provided herein are methods comprising contacting a pool of polynucleotides with a library of beads, wherein the pool of polynucleotides comprises polynucleotide sets P11, . . . , and P1j₁; . . . ; Pk1, . . . , and Pkjₖ; . . . ; and Pi1, . . . , and Pijᵢ, wherein i, j₁, . . . , jₖ, . . . , jᵢ, and k are integers, i, j₁, . . . , jₖ, . . . , and jᵢ are independently 2 or greater, and 1≤k≤i, Pk1, . . . , and Pkjₖ comprise subsequences Sk1, . . . , and Skjₖ, respectively, which form target sequence S′k, at least one of Pk1, . . . , and Pkjₖ comprises, in the 3′ to 5′ direction (i) a single-stranded 3′ end sequence, (ii) the subsequence of target sequence S′k, (iii) a Type IIS restriction enzyme recognition sequence, and (iv) a complementary sequence capable of hybridizing to all or a portion of the subsequence of target sequence S′k, the at least one of Pk1, . . . , and Pkjₖ further comprises a tag Tk in all or a subset of Pk1, . . . , and Pkjₖ, and the at least one of Pk1, . . . , and Pkjₖ is capable of forming a hairpin molecule comprising a 3′ overhang, a stem formed by intramolecular nucleotide base pairing between all or a portion of the subsequence of target sequence S′k and the complementary sequence, and a loop comprising the Type IIS restriction enzyme recognition sequence in a configuration that is not cleaved by a Type IIS restriction enzyme; beads B1, . . . , Bk, . . . , and Bi in the library comprise capture moieties C1, . . . , Ck, . . . , and Ci, respectively, that specifically binds to tags T1, . . . , Tk, . . . , and Ti, respectively, thereby specifically capturing the at least one of Pk1, . . . , and Pkjₖ on one of the beads in the library.

In some embodiments, the methods can further comprise placing all or a subset of the beads in emulsion droplets, e.g., one bead per emulsion droplet. In some embodiments, the distribution of beads in the emulsion droplets is a random distribution, wherein on average the droplets contain either no beads or one bead per droplet, and few contain two or more beads. In some embodiments, the distribution of beads in the emulsion droplets is a Poisson distribution. In some embodiments, as a result of the partitioning, at least about 75%, at least about 80%, at least about 85%, at least about 90%, at least about 95%, at least about 96%, at least about 97%, at least about 98%, at least about 99%, or about 100% of the droplets contain either no beads or one bead per droplet. In some embodiments, at least about 90%, at least about 95%, at least about 96%, at least about 97%, at least about 98%, at least about 99%, or about 100% of the droplets contain one bead per droplet.

In some embodiments, a pool of hairpin addition oligos can comprise oligo sets:

| | |
|---|---|
| Set 1: | P11, . . . , and P1j₁; |
| . . . ; | |
| Set k: | Pk1, . . . , and Pkjₖ; |
| . . . ; | |
| Set m: | Pm1, . . . , and Pmjₘ; |
| . . . ; and | |
| Set i: | Pi1, . . . , and Pijᵢ, |

wherein i, j₁, . . . , jₖ, . . . , jₘ, . . . , jᵢ, k, and m are integers, i, j₁, . . . , jₖ, . . . , jₘ, . . . , and jᵢ are independently 2 or greater, 1≤k≤i, and 1≤m≤i,

wherein oligos Pk1, . . . , and Pkjₖ comprise subsequences Sk1, . . . , and Skjₖ, respectively, which form target sequence S′k, and wherein Pk1, . . . , and Pkjₖ further comprise a common capture tag sequence Tk, and

wherein oligos Pm1, . . . , and Pmjₘ comprise subsequences Sm1, . . . , and Smjₘ, respectively, which form target sequence S′m, and wherein Pm1, . . . , and Pmjₘ further comprise a common capture tag sequence Tm.

In some embodiments, beads B1, . . . , Bk, . . . , Bm, . . . , and Bi in the library comprise capture oligos C1, . . . , Ck, . . . , Cm, . . . , and Ci, respectively, that specifically hybridizes to tags T1, . . . , Tk, . . . , Tm, . . . , and Ti, respectively, thereby specifically capturing oligo set 1, . . . , oligo set k, . . . , oligo set m, . . . , and oligo set i onto a bead B1, . . . , Bk, . . . , Bm, . . . , and Bi, respectively, in the bead library.

In some embodiments, provided herein is a barcoded bead library comprising different types of beads, e.g., bead(s) Bk and bead(s) Bm comprising capture oligos Ck and Cm respectively. In some embodiments, the capture oligos on different types of beads specifically hybridize to different tags, thereby specifically capturing an oligo set on one type of beads in the barcoded bead library. In some embodiments, the number of different types of beads in the library is 2, 3, 4, 5, 6, 7, 8, 9, at least 10, at least 50, at least 100, or any range between the foregoing. In some embodiments, the number of different types of beads in the library is from about 2 to about 4, about 4 to about 8, about 8 to about 16, about 16 to about 32, about 32 to about 64, or about 64 to about 128, or more.

In some embodiments, a partition (e.g., an emulsion droplet) comprises two or more beads. The two or more beads can of the same “type” of different types. For example, an emulsion droplet can comprise two or more beads Bk, both or all of which have the same oligos from set k captured thereon. In these examples, the assembled products are the same as those generated in an emulsion droplet having only one bead Bk.

In other examples, an emulsion droplet can comprise one or more beads Bk and one or more beads Bm, and after releasing the oligos, the emulsion droplet may comprise oligo set k and oligo set m. In some embodiments, assembly of oligos in set k to form target sequence S′k and assembly of oligos in set m to form target sequence S′m proceed in the same partition without interfering with each other, e.g., each in a predetermined order of adding the oligos based on sequence complementarity between the 3′ overhang of an addition oligo and a 3′ overhang of a cleaved assembly product from the previous cycle. In some embodiments, assembled products in a partition are detected, analyzed, and/or selected, e.g., in order to separate correctly assembled molecules (e.g., molecules comprising either S′k or S′m) from assembled molecules containing one or more errors, including assembly errors due to two different types of beads being in the same emulsion droplet, such as a single molecule comprising both a sequence from set k and a sequence from set m.

In some embodiments, the methods can further comprise releasing all or a subset of the polynucleotides captured on each of all or a subset of the beads in the emulsion droplets. In some embodiments, the methods can further comprise within each emulsion droplet, connecting two or more of Pk1, . . . , and Pkjₖ, thereby assembling two or more of subsequences Sk1, . . . , and Skjₖ, in the emulsion droplet.

In some embodiments, Pk1, . . . , and Pkjₖ can be assembled in the emulsion droplet by one or more concerted reaction cycles. In any of the preceding embodiments, one reaction cycle can comprise sequential reactions comprising hybridization, ligation, primer extension, and/or cleavage of an assembled product, and the sequential reactions can be repeated one or more times to add a polynucleotide (e.g., a hairpin addition oligo) to the cleaved assembled product from the previous cycle. In some embodiments, the one or more concerted reaction cycles can comprise an isothermal reaction. In any of the preceding embodiments, the one or more concerted reaction cycles can comprise sequential reactions of hybridization, ligation by a ligase, primer extension by a polymerase, and cleavage by a Type IIS restriction enzyme. In any of the preceding embodiments, the one or more concerted reaction cycles can comprise sequential assembly of all or a subset of Pk1, . . . , and Pkjₖ in a predetermined order.

In any of the preceding embodiments, subsequence sets S11, . . . , and S1j₁; . . . ; Sk1, . . . , and Skjₖ; . . . ; and Si1, . . . , and Sijᵢ can comprise one or more common subsequences among two or more of the subsequence sets. In any of the preceding embodiments, polynucleotide sets P11, . . . , and P1j₁; . . . ; Pk1, . . . , and Pkjₖ; . . . ; and Pi1, . . . , and Pijᵢ can comprise one or more common polynucleotides among two or more of the polynucleotide sets.

In any of the preceding embodiments, subsequence sets S11, . . . , and S1j₁; . . . ; Sk1, . . . , and Skjₖ; . . . ; and Si1, . . . , and Sijᵢ may not contain a common subsequence.

In any of the preceding embodiments, Pk1, . . . , and Pkjₖ can be assembled to form target sequence S′k or a portion thereof. In any of the preceding embodiments, polynucleotide sets P11, . . . , and P1j₁; . . . ; Pk1, . . . , and Pkjₖ; . . . ; and Pi1, . . . , and Pijᵢ can be assembled to form target sequences S′1, . . . , S′k, . . . , and S′i or a portion thereof, respectively, in parallel.

In any of the preceding embodiments, the methods can further comprise breaking the emulsion droplets and pooling all or a subset of the assembled target sequences or portions thereof. In any of the preceding embodiments, all or a subset of the assembled target sequences or portions thereof can be subjected to further assembly. In some embodiments, the further assembly can comprise higher order assembly of all or a subset of the assembled target sequences or portions thereof. In any of the preceding embodiments, the further assembly can comprise polymerase cycling assembly (PCA), sequence- and ligation-independent cloning (SLIC), Golden Gate assembly, Gibson assembly, in vivo assembly, or any combination thereof.

In any of the preceding embodiments, the target sequence can comprise a sequence difficult to synthesize, difficult to amplify, and/or difficult to sequence verify. In any of the preceding embodiments, the target sequence can comprise a sequence difficult to synthesize base-by-base. In any of the preceding embodiments, the target sequence can comprise a homopolymer sequence, e.g., Aₙ; a homocopolymer sequence, e.g., [AT]ₙ; a sequence comprising direct repeats; an AT-rich sequence; a GC-rich sequence, or any combination thereof.

### BRIEF DESCRIPTION OF THE DRAWINGS

FIGS. 1A-1I illustrate a non-limiting exemplary method of serial multiplexed polynucleotide synthesis showing serial addition of subsequences to form a target nucleic acid sequence. FIG. 1A shows an exemplary seed oligonucleotide and an exemplary addition oligonucleotide. In some embodiments, the seed oligonucleotide comprises two different ends. For example, the two 3′ overhangs of the exemplary seed oligonucleotide can have different sequences. Such different 3′ overhang sequences are useful for unidirectional addition (e.g., oligos are added to one 3′ overhang but not the other 3′ overhang due to differences in sequences) or bidirectional addition (e.g., oligos having different 3′ overhang sequences are added to different 3′ overhangs of the seed oligo). FIG. 1B shows an exemplary addition oligonucleotide comprising a useful sequence (e.g., one or more adapter, tag, primer binding, cleavage, UMI/UID, and/or barcode sequences) between the subsequence and the complementary sequence, and the addition oligonucleotide may be captured by a capture oligo immobilized on a support (e.g., a bead), through hybridization between a sequence of the capture oligo and a sequence of the addition oligonucleotide. FIG. 1C shows an exemplary pool of addition oligonucleotides in a container such as a vial. FIG. 1D shows, in an exemplary method, that a pool of addition oligos A, B, C, and D are contacted with beads having a capture oligo C1′ or C2′ which is capable of hybridizing to capture tag sequences C1 and C2, respectively. FIG. 1E shows that beads comprising only capture oligo C1′ are capable of capturing hairpin oligos A and B, both of which comprise capture tag sequence C1, while hairpin oligos C and D comprising capture tag sequence C2 are specifically captured on beads comprising only capture oligo C2′. FIG. 1F shows, in an exemplary method, that beads with Oligo A and Oligo B captured thereon and beads with Oligo C and Oligo D captured thereon are partitioned into a plurality of partitions, e.g., emulsion droplets. FIG. 1G shows that the captured oligos may be released from the beads, and without breaking the emulsions, a reaction assembling Oligos A and B (and optionally other oligos) into the first target sequence and a reaction assembling Oligos C and D (and optionally other oligos) into the second target sequence may proceed in separate emulsion droplets in parallel and without interfering with each other. FIG. 1H shows exemplary assembled products after the partitions are combined. FIG. 1I shows exemplary assembled products comprising one or more useful moieties sequences provided by a seed oligo and/or a terminal sequence, and that the exemplary assembled products can be amplified, e.g., by using one or more PCR primers that bind to the one or more useful sequences.

FIG. 2 shows exemplary seed oligos that can be used in assembling a target polynucleotide. The seed oligo may consist of a single nucleic acid strand (FIG. 2, first row, a hairpin addition oligo is shown to illustrate hybridization) or comprise two nucleic acid strands (FIG. 2, second row, a hairpin addition oligo is shown to illustrate hybridization). In any of the embodiments disclosed herein, a 5′ end nucleotide of a seed oligo may be blocked, e.g., dephosphorylated to prevent ligation. In any of the embodiments disclosed herein, a seed oligo may comprise a useful sequence. In any of the embodiments disclosed herein, a seed oligo may be immobilized on a support, e.g., a bead or a solid substrate. In any of the embodiments disclosed herein, a seed oligo may comprise a hairpin, for example, as a blocker to ligation. A seed oligo may comprise any two or more features disclosed herein combined in a suitable manner. For example, a seed oligo may be provided as separate components, such as a useful sequence immobilized on a bead and a double-stranded oligo that hybridizes to the useful sequence. In another example, two or more (e.g., 4) nucleic acid strands may form a hybridization complex and provide a seed oligo having two or more (e.g., 4) 3′ end overhangs as shown in the figure.

FIG. 3A shows exemplary hairpin molecules that can be used as seed and/or addition oligos in assembling a target polynucleotide. FIG. 3B shows exemplary hairpin molecules that comprise one or more bulges in one or more strands of the stem of a primary hairpin. FIG. 3C shows exemplary arrangements of the restriction enzyme recognition sequence relative to one or more useful moieties (e.g., sequences), e.g., an adapter, a tag, a primer binding moiety, a cleavage site, a UMI/UID, and/or a barcode.

FIG. 4A shows an exemplary target polynucleotide that can be assembled from five subsequences, and exemplary polynucleotides (e.g., oligos) for use during a first cycle of assembling (e.g., using a seed oligo and an addition oligo). The figure also shows exemplary hairpin oligos for use during subsequent cycles of addition.

FIG. 4B shows seed and addition oligos may be designed to assemble subsequences into a circular double-stranded target polynucleotide.

FIGS. 5A-5E show exemplary target polynucleotides to be assembled (top), and supports (e.g., beads or solid substrates) that can be used to capture oligos such as hairpin molecules by their tag sequences, for assembling subsequences in the oligos to form one or more target sequences.

FIG. 6A shows an exemplary method of using a support (e.g., bead or solid substrate) to capture polynucleotides for unidirectional assembly of a target polynucleotide.

FIG. 6B shows an exemplary method comprising Cycle 1 reactions where a single-stranded polynucleotide is not attached to a support (e.g., bead or solid substrate), and a hairpin molecule comprises a 3′ overhang capable of hybridizing to a 3′ sequence of the single-stranded polynucleotide.

FIG. 6C and FIG. 6D show the first and second cycle, respectively, of an exemplary method of assembling a target polynucleotide.

FIG. 7A and FIG. 7B show the first and second cycle, respectively, of an exemplary method of assembling a target polynucleotide.

FIG. 8A and FIG. 8B show the first and second cycle, respectively, of an exemplary method of assembling a target polynucleotide.

FIG. 9 shows the first cycle of an exemplary method of assembling a target polynucleotide. Cycle 2 and subsequent cycles of assembly can proceed essentially as described for FIG. 6D.

FIG. 10 shows an exemplary method comprising consecutive levels of assembly using sequential addition of hairpin oligos.

FIG. 11 shows an exemplary method comprising a first level and a second level of assembly and optionally even higher levels of assembly.

### DETAILED DESCRIPTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W. H. Freeman, New York N.Y.; Gait, “Oligonucleotide Synthesis: A Practical Approach” 1984, IRL Press, London; Nelson and Cox (2000), Lehninger, Principles of Biochemistry 3rd Ed., W. H. Freeman Pub., New York, N.Y.; and Berg et al. (2002) Biochemistry, 5^{th} Ed., W. H. Freeman Pub., New York, N.Y., all of which are herein incorporated in their entirety by reference for all purposes. Other suitable techniques can be had by reference to U.S. Pat. Nos. 4,500,707, 4,683,195, 4,683,202, 4,689,405, 4,725,677, 4,800,159, 4,965,188, 4,999,294, 5,047,524, 5,104,789, 5,104,792, 5,132,215, 5,143,854, 5,288,514, 5,356,802, 5,384,261, 5,405,783, 5,424,186, 5,436,150, 5,436,327, 5,445,934, 5,459,039, 5,474,796, 5,498,531, 5,508,169, 5,510,270, 5,512,463, 5,514,789, 5,527,681, 5,541,061, 5,605,793, 5,624,711, 5,639,603, 5,641,658, 5,653,939, 5,674,742, 5,679,522, 5,695,940, 5,700,637, 5,700,642, 5,702,894, 5,738,829, 5,739,386, 5,750,335, 5,766,550, 5,770,358, 5,780,272, 5,795,714, 5,830,655, 5,830,721, 5,834,252, 5,858,754, 5,861,482, 5,871,902, 5,877,280, 5,916,794, 5,922,539, 5,928,905, 5,929,208, 5,942,609, 5,953,469, 6,008,031, 6,013,440, 6,017,696, 6,042,211, 6,093,302, 6,103,463, 6,136,568, 6,150,102, 6,150,141, 6,165,793, 6,177,558, 6,242,211, 6,248,521, 6,261,797, 6,271,957, 6,277,632, 6,280,595, 6,284,463, 6,287,825, 6,287,861, 6,291,242, 6,315,958, 6,322,971, 6,333,153, 6,346,399, 6,358,712, 6,365,355, 6,372,434, 6,372,484, 6,375,903, 6,406,847, 6,410,220, 6,416,164, 6,426,184, 6,444,111, 6,444,175, 6,479,652, 6,480,324, 6,489,146, 6,495,318, 6,506,603, 6,511,849, 6,514,704, 6,521,427, 6,534,271, 6,537,776, 6,565,727, 6,586,211, 6,596,239, 6,605,451, 6,610,499, 6,613,581, 6,632,641, 6,650,822, 6,658,802, 6,664,112, 6,664,388, 6,670,127, 6,670,605, 6,800,439, 6,802,593, 6,824,866, 6,830,890, 6,833,450, 6,846,655, 6,897,025, 6,911,132, U.S. Pat. Nos. 6,921,818, 6,932,097, 6,969,587, 6,969,847, 7,090,333, 7,133,782, 7,169,560, 7,179,423, 7,183,406, 7,262,031, 7,273,730, 7,303,320, 7,303,872, 7,323,320, 7,399,590, 7,432,055, 7,498,176, 7,563,600, 7,820,412, 7,879,580, 8,053,191, 8,058,004, 8,173,368, 8,716,467, 8,808,986, 9,023,601, 9,051,666, 9,217,144, 9,403,141, 9,555,388, 9,677,067, 9,833,761, 9,839,894, 9,889,423, 9,895,673, 9,925,510, 9,981,239, 10,053,688, 10,053,719, 10,202,628, 10,272,410, 10,273,471, 10,384,188, 10,384,189, 10,417,457, 10,583,415, 10,618,024, 10,632,445, 10,639,609, 10,669,304, 10,696,965, 10,744,477, 10,754,994, US 2001/0012537, US 2001/0031483, US 2001/0049125, US 2002/0012616, US 2002/0037579, US 2002/0058275, US 2002/0081582, US 2002/0127552, US 2002/0132259, US 2002/0132308, US 2002/0133359, US 2003/0017552, US 2003/0044980, US 2003/0047688, US 2003/0050437, US 2003/0050438, US 2003/0054390, US 2003/0068633, US 2003/0068643, US 2003/0082630, US 2003/0087298, US 2003/0091476, US 2003/0099952, US 2003/0118485, US 2003/0118486, US 2003/0120035, US 2003/0134807, US 2003/0143550, US 2003/0143724, US 2003/0170616, US 2003/0171325, US 2003/0175907, US 2003/0186226, US 2003/0198948, US 2003/0215837, US 2003/0215855, US 2003/0215856, US 2004/0002103, US 2004/0005673, US 2004/0009479, US 2004/0009520, US 2004/0014083, US 2004/0101444, US 2004/0101894, US 2004/0101949, US 2004/0106728, US 2004/0110211, US 2004/0110212, US 2004/0126757, US 2004/0132029, US 2004/0166567, US 2004/0171047, US 2004/0185484, US 2004/0241655, US 2004/0259146, US 2005/0053997, US 2005/0069928, US 2005/0079510, US 2005/0106606, US 2005/0118628, US 2005/0202429, US 2005/0227235, US 2005/0255477, US 2006/0008833, US 2006/0040297, US 2006/0054503, US 2006/0127920, US 2006/0127926, US 2006/0134638, US 2006/0160138, US 2006/0194214, US 2007/0004041, US 2007/0122817, US 2007/0231805, US 2007/0281309, US 2007/0292954, US 2008/0009420, US 2008/0014589, US 2008/0105829, US 2008/0274513, US 2008/0300842, US 2009/0016932, US 2009/0137408, US 2009/0311713, US 2009/0878840, US 2010/0015614, US 2010/0015668, US 2010/0016178, US 2011/0008775, US 2011/0117625, US 2012/0028843, US 2012/0220497, US 2012/0270754, US 2012/0283110, US 2012/0283140, US 2012/0315670, US 2012/0322681, US 2013/0059296, US 2013/0059761, US 2013/0244884, US 2013/0252849, US 2013/0281308, US 2013/0296192, US 2013/0296194, US 2013/0309725, US 2014/0141982, US 2014/0309119, US 2015/0065393, US 2015/0159204, CN 100510069, CN 101560538, CN 104212791, EP 0259160, EP 1015576, EP 1159285, EP 1180548, EP 1205548, WO 1990/000626, WO 1993/017126, WO 1993/020092, WO 1994/018226, WO 1997/035957, WO 1998/005765, WO 1998/020020, WO 1998/038326, WO 1999/019341, WO 1999/025724, WO 1999/042813, WO 2000/029616, WO 2000/040715, WO 2000/046386, WO 2000/049142, WO 2001/088173, WO 2002/004597, WO 2002/024597, WO 2002/081490, WO 2002/095073, WO 2002/101004, WO 2003/010311, WO 2003/033718, WO 2003/040410, WO 2003/046223, WO 2003/054232, WO 2003/060084, WO 2003/064026, WO 2003/064027, WO 2003/064611, WO 2003/064699, WO 2003/065038, WO 2003/066212, WO 2003/100012, WO 2004/002627, WO 2004/024886, WO 2004/029586, WO 2004/031351, WO 2004/031399, WO 2004/034028, WO 2004/090170, WO 2005/059096, WO 2005/071077, WO 2005/089110, WO 2005/107939, WO 2005/123956, WO 2006/044956, WO 2006/049843, WO 2006/076679, WO 2006/127423, WO 2007/008951, WO 2007/009082, WO 2007/075438, WO 2007/087347, WO 2007/113688, WO 2007/117396, WO 2007/120624, WO 2007/123742, WO 2007/136736, WO 2007/136833, WO 2007/136834, WO 2007/136835, WO 2007/136840, WO 2008/024319, WO 2008/045380, WO 2008/054543, WO 2008/076368, WO 2008/130629, WO 2010/025310, WO 2011/056872, WO 2011/066185, WO 2011/066186, WO 2011/085075, WO 2012/024351, WO 2012/064975, WO 2012/078312, WO 2012/103154, WO 2012/174337, WO 2013/032850, WO 2013/163263, WO 2014/004393, WO 2014/151696, WO 2014/160004, and WO 2014/160059, all of which are herein incorporated in their entirety by reference for all purposes.

Synthesis of large numbers of long polynucleotides quickly and inexpensively, e.g., using chemical synthesis, is of significant interest for a wide range of applications. Such exemplary applications include the synthesis of synthetic clones directly from genomic sequence data, the synthesis of large gene libraries, the synthesis of chromosomes, including natural or artificial chromosomes or fragments thereof, and the synthesis of entire native or synthetic genomes.

Aspects of the present disclosure relate to methods and compositions for designing and producing a target nucleic acid. In particular, aspects of the present disclosure relate to the multiplex and/or parallel synthesis of target polynucleotides. Some or all of the target polynucleotides can have the same sequence or substantially identical sequences, and some or all of the target polynucleotides can have different sequences.

In some aspects, provided herein are methods and compositions to isolate, co-locate, and/or enrich one or more oligonucleotide sequences (e.g., DNA and/or RNA sequences) from a pool of oligonucleotide sequences and create assembled nucleic acid sequences of interest (e.g., DNA and/or RNA sequences (e.g., genes, genomes and the like)). In some embodiments, the one or more oligonucleotide sequences are isolated, co-located, or enriched within a partition, such as an emulsion droplet. In some embodiments, assembled nucleic acid molecules are created within the partition, e.g., a plurality of emulsion droplets may be used to assemble target nucleic acid molecules in parallel. In some embodiments, methods are provided to create long synthetic nucleic acid pools or gene libraries using short nucleic acids such as oligonucleotides which may be produced of obtained from plates or arrays of synthetic oligonucleotides. In some embodiments, amplification and/or assembly of nucleic acid sequences is carried out using bead based emulsions. Further provided herein are methods for generating oligonucleotide molecules, such as seed constructs (e.g., seed oligos), addition constructs (e.g., addition oligos), terminal constructs (e.g., terminal oligos), capture constructs (e.g., capture oligos immobilized on a support), and primers, that are useful for synthesizing one or more nucleic acid sequences of interest (e.g., gene(s), genome(s) and the like). Further provided herein are barcodes and a barcoded library, such as a barcoded bead library, for use in the methods described herein.

In some embodiments, use of a site-specific “outside cutter” endonuclease (e.g., Type IIS restriction enzymes) produces cleavage sites adjacent to the enzyme recognition sites, typically indiscriminative of the nucleotide content of the sequence between the enzyme recognition site and the cleavage sites. In some embodiments, the cleavage site is non-overlapping with the enzyme recognition site. Thus, each overhang (e.g., 3′ overhang) created by the cleavage would have sequence specific to that part of the DNA, distinct from that of the other sites. Two segments may be designed to have or form the specifically complementary cohesive ends that can bring the two segments together in the proper order. For instance, when the cohesive ends generated are five bases in length, up to 4⁵=1024 different combinations can be generated. When the cohesive ends generated are four bases in length, up to 4⁴=256 different combinations can be generated. When the cohesive ends generated are three bases in length, up to 4³=64 different combinations can be generated. When the cohesive ends generated are two bases in length, up to 4²=16 different combinations can be generated. The necessary restriction sites can be specifically included in the design of the sequence.

In some embodiments, self-complementary sequences are avoided, since an addition oligo with a self-complementary sequence at the 3′ overhang could anneal to itself. Exemplary self-complementary sequences include AT/TA, GC/CG, or longer self-complementary sequences. In some embodiments, self-complementary sequences may include GT/TG since G and T can also form a base pair. In some embodiments, self-complementary sequences are avoided in a 3′ sequence (e.g., 3′ overhang) of a seed oligo, e.g., when one end of the seed oligo is not immobilized on a support or otherwise protected or blocked from annealing to another molecule of the same seed oligo. In some embodiments, a 3′ sequence (e.g., 3′ overhang) of a seed oligo may comprise a self-complementary sequence, e.g., when one end of the seed oligo is immobilized on a support or otherwise protected or blocked to prevent annealing among seed oligo molecules. In some embodiments, after excluding four self-complementary sequences AT/TA and GC/CG, 12 different combinations of two-base cohesive ends may be used in designing 3′ overhang sequences of addition oligos. In some embodiments, after further excluding GT/TG, 10 different combinations of two-base cohesive ends may be used in designing 3′ overhang sequences of addition oligos.

In some embodiments, the cohesive end sequences (e.g., the 16, 12, or 10 different combinations of two-base cohesive ends) are part of a target sequence. In some embodiments, designing oligos comprising these sequences comprises choosing which sticky end to use out of the options (e.g., two-base sequences) available in a target sequence, and the location of the cut site can be fine-tuned. In some embodiments, a target sequence contains all of the two-base sequences needed to design oligos for assembling the target sequence, without the need to alter the target sequence, e.g., by adding extra sequences and/or deleting sequences.

Aspects of the present disclosure can be used to assemble large numbers of nucleic acid fragments efficiently, and/or to reduce the number of steps required to generate large nucleic acid products, while reducing assembly error rate. In some embodiments, methods and compositions disclosed herein can be incorporated into nucleic assembly procedures to increase assembly fidelity, throughput and/or efficiency, decrease cost, and/or reduce assembly time. In some embodiments, the methods may be automated and/or implemented in a high throughput assembly context to facilitate parallel production of many different target nucleic acid products.

In some embodiments, provided herein are methods and compositions for the selection, localization, and/or enrichment of one or more sets of oligonucleotides comprising subsequences from among a plurality of oligonucleotides comprising subsequences, such as a mixture of oligonucleotides comprising subsequences. In some embodiments, each set of the one or more sets of oligonucleotides comprising subsequences is used to assemble one or more assembled nucleic acid sequences. Accordingly, one aspect is directed to assembly of one or more nucleic acid sequences of interest from a large pool of oligonucleotide sequences.

In some embodiments, a set of oligonucleotides comprising subsequences is partitioned into a partition. In some embodiments, a set of oligonucleotides comprising subsequences is sequestered, localized, contained within an emulsion droplet. In some embodiments, a plurality of emulsion droplets is provided with each including a set of subsequence oligonucleotides. In some embodiments, the emulsion droplet includes the set of subsequence oligonucleotides and reagents sufficient to assemble the subsequence oligonucleotides into one or more assembled nucleic acid sequences.

In some embodiments, oligonucleotides each comprising one or more subsequences of a target sequence collectively forming an oligonucleotide set are localized (e.g., captured) by hybridization to one or more predesigned sequences (e.g., barcode sequences) for each oligonucleotide set. In some embodiments, the one or more predesigned sequences are unique to each oligonucleotide set. The oligonucleotide set can correspond to a particular target nucleic acid sequence. The captured oligonucleotide set can be assembled into an assembled nucleic acid sequence, such as an assembled target nucleic acid sequence. In some embodiments, the captured oligonucleotide set can be attached to a bead. The bead can then be sequestered or contained within an emulsion droplet, e.g., through one or more levels of partitioning. The oligonucleotide set can then be detached or released from the bead and contained within the emulsion droplet. The released oligonucleotide set within the emulsion droplet can then be assembled into one or more assembled nucleic acid sequences in the presence of suitable reagents within the emulsion droplet and with the emulsion droplet under suitable reaction conditions.

In some embodiments, one or more seed oligos and one or more addition oligos share a common capture tag sequence, which can be a barcode specific to a set of oligos for assembling a target nucleic acid sequence. In some embodiments, one or more seed oligos and addition oligos necessary to create a target nucleic acid sequence are in a solution, and can be pulled down onto a bead. The beads can then be emulsified with no more than one bead being contained within a single emulsion droplet. In some embodiments, the beads are partitioned such that on average the droplets contain one bead per droplet (e.g., the distribution of the number of beads in each droplet is a Poisson distribution), and there may be droplets that contain no bead and droplets containing two or more beads. In some embodiments, the beads are partitioned such that no more than about 25%, no more than about 20%, no more than about 15%, no more than about 10%, no more than about 5%, no more than about 4%, no more than about 3%, no more than about 2%, no more than about 1%, no more than about 0.5%, or no more than about 0.1% of the droplets contain two or more beads per droplet. In some embodiments, at least 90%, 91%, 92%, 93%, 94%, 95%, 96%, 97%, 98%, 99%, or 100% of the droplets contain one bead per droplet. In some embodiments, assembled products in a droplet containing two or more beads can be detected, analyzed, and/or selected, e.g., in order to separate correctly assembled molecules from assembled molecules containing one or more errors, including errors due to two different types of beads being in the same emulsion droplet.

The captured oligos can be released from a bead in an emulsion droplet, e.g., using heating and/or enzyme cleavage. The freed or detached oligonucleotides within the emulsion droplet are then assembled within the emulsion droplet by concerted reactions that involve hybridization based on sequence complementarity, ligation (e.g., by a high fidelity ligase such as a thermostable DNA ligases, including a Taq DNA ligase), primer extension by a polymerase (e.g., a high fidelity polymerase, including DNA polymerases such as a Taq DNA polymerase, Phusion® High-Fidelity DNA Polymerase, KAPA Taq, KAPA Taq HotStart DNA Polymerase, KAPA HiFi, and/or Q5® High-Fidelity DNA Polymerase), and/or cleavage by a restriction enzyme such as a Type IIS enzyme. Oligos comprising oligos capable of forming hairpin structures are added sequentially, e.g., in a predetermined order, in order to generate one or more assembled nucleic acid sequences. The emulsion droplets are broken and the assembled constructs are collected thereby resulting in large libraries of assembled constructs.

Aspects of the technology provided herein are useful for increasing the accuracy, yield, throughput, and/or cost efficiency of nucleic acid synthesis and assembly reactions.

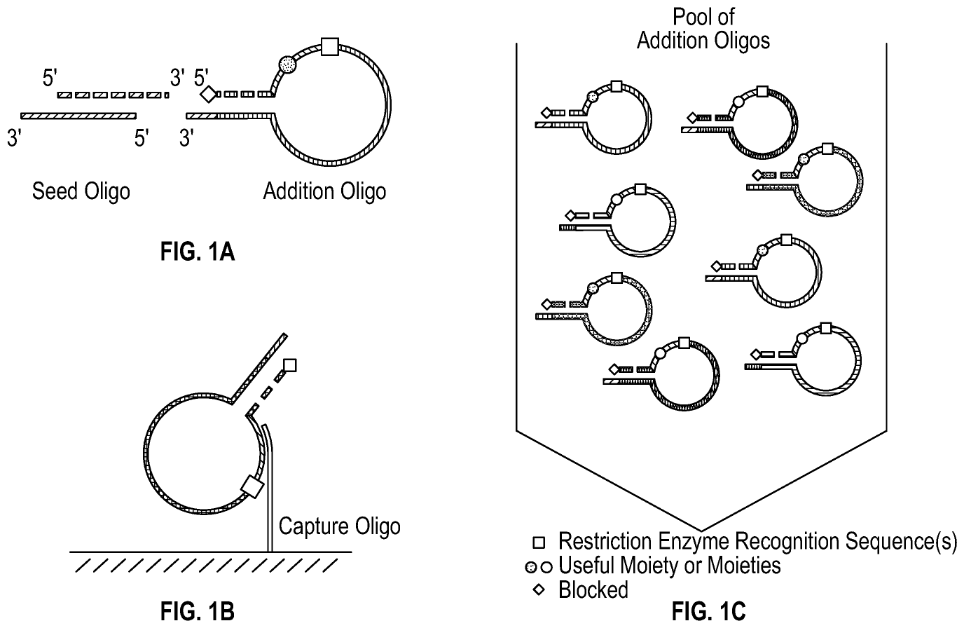

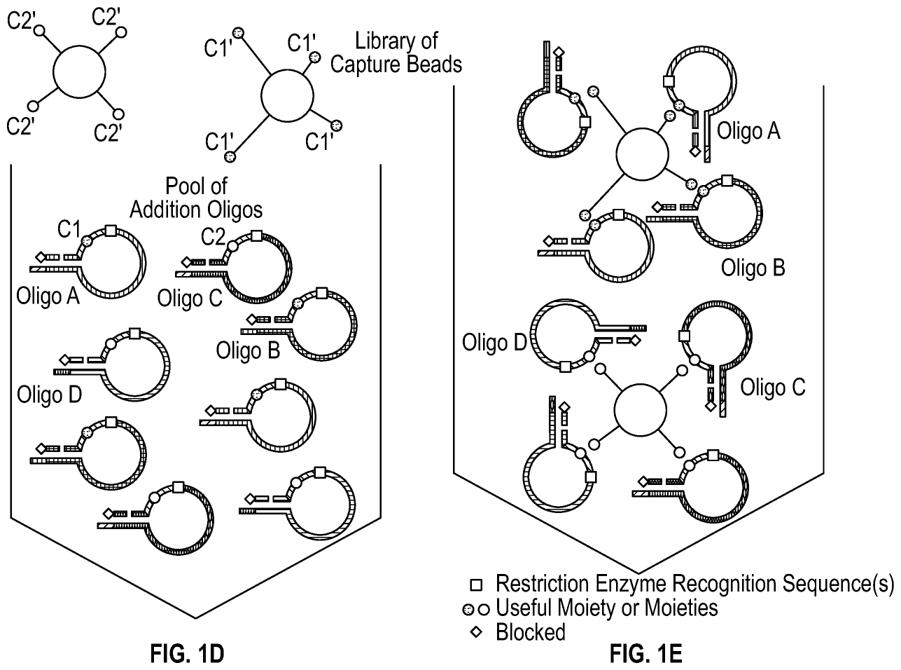

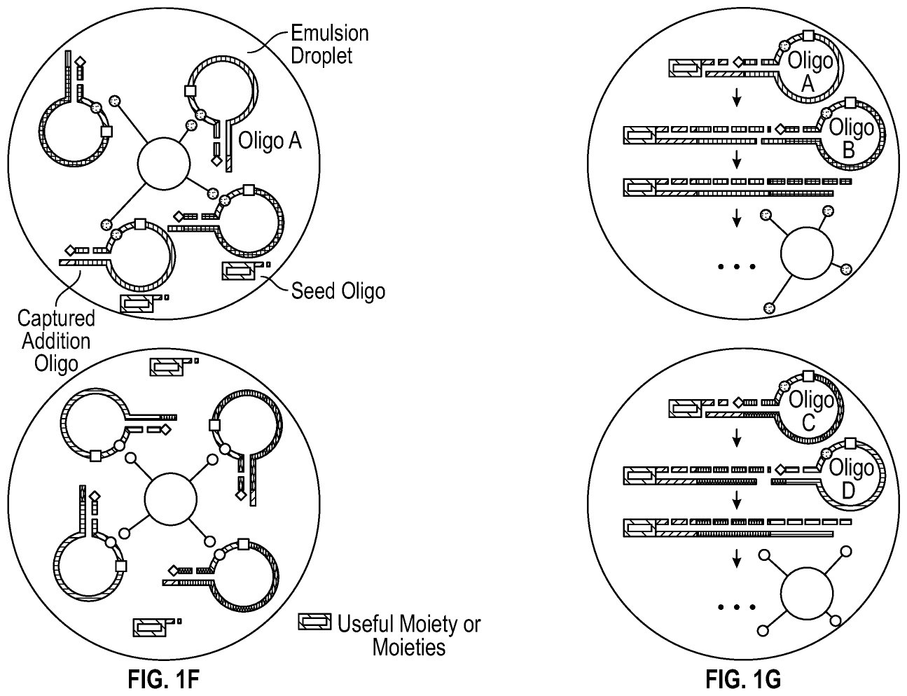

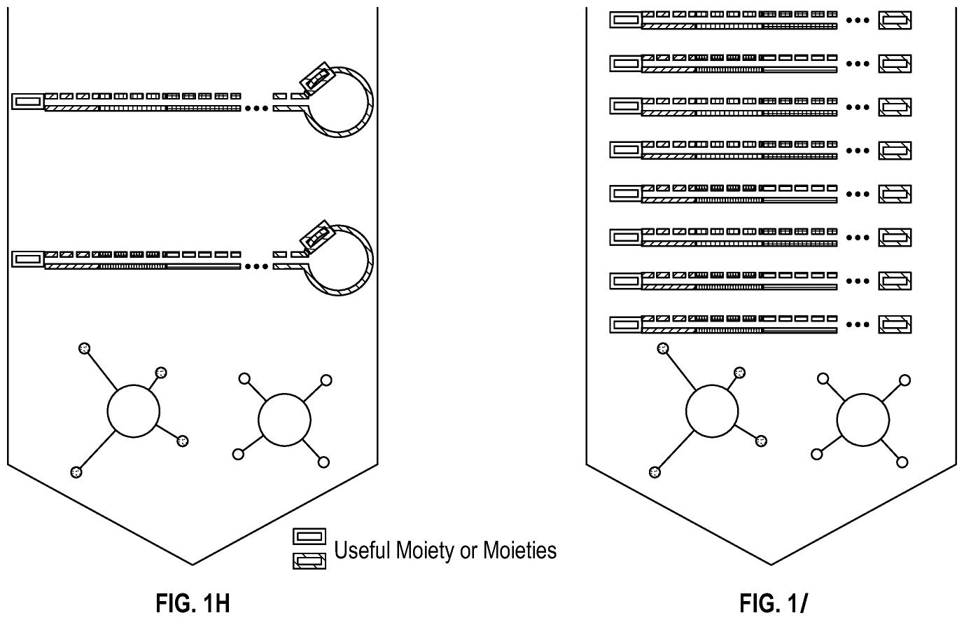

Turning to the figures, FIGS. 1A-1I illustrate non-limiting exemplary methods of serial multiplexed polynucleotide synthesis showing serial addition of subsequences to form a target nucleic acid sequence, e.g., using one or more seed oligonucleotides and a plurality of exemplary addition oligonucleotides.

In FIG. 1A, the exemplary seed oligonucleotide comprises at least one 3′ single-stranded overhang that is capable of hybridizing to a 3′ single-stranded overhang of the addition oligonucleotide. The seed oligonucleotide is shown as a duplex comprising two 3′ single-stranded overhangs as an example, and can be single-stranded or double-stranded (e.g., having one blunt end), have one or two free 3′ ends, and/or be immobilized or not immobilized on a support. The 5′ end of the strand having the at least one 3′ single-stranded overhang (e.g., top strand of the seed oligonucleotide in FIG. 1A) may be blocked or have a phosphate group that permits ligation, while the 3′ end generally permits ligation and/or primer extension. The 3′ end of the other strand (e.g., bottom strand of the seed oligonucleotide in FIG. 1A) may be blocked or have a hydroxyl group that permits ligation and/or primer extension, while the 5′ end generally permits ligation but in certain examples may be blocked (e.g., primer extension by a polymerase of the 3′ end of the addition oligonucleotide may displace the blocked bottom strand). The seed oligonucleotide may or may not comprise a subsequence of a target sequence to be assembled, and may be a common seed oligo shared by all or a subset of a plurality of addition oligos.

As shown in FIG. 1A, the exemplary addition oligonucleotide generally is capable of forming a hairpin structure having a 3′ single-stranded overhang; a subsequence to become part of a target sequence; one or more restriction enzyme recognition sequences; a complementary sequence to a 3′ sequence of the subsequence; and/or one or more adapter, tag, primer binding, cleavage, UMI/UID, and/or barcode sequences. The 5′ end of the addition oligonucleotide is generally blocked (e.g., dephosphorylated) from ligation, but in certain addition oligonucleotides (e.g., the last addition oligonucleotide in a serial addition), the 5′ ends may permit ligation. Once the seed oligonucleotide and the addition oligonucleotide hybridize to each other, the 3′ end of the addition oligonucleotide may be ligated to the 5′ end of the bottom strand of the seed oligonucleotide (with or without primer extension prior to ligation), while the 3′ end of the top strand of the seed oligonucleotide is generally not ligated to the 5′ end of the addition oligonucleotide but may be extended by a polymerase using the addition oligonucleotide as a template.

The exemplary addition oligonucleotide in FIG. 1B comprises a useful sequence (e.g., one or more adapter, tag, primer binding, cleavage, UMI/UID, and/or barcode sequences) between the subsequence and the complementary sequence. The addition oligonucleotide may be captured by a capture oligo immobilized on a support (e.g., a bead), through hybridization between a sequence of the capture oligo and a sequence (e.g., the useful sequence) of the addition oligonucleotide. The addition oligonucleotide may be released from the support, e.g., by heating the hybridization complex. In some embodiments, the support is a bead, and a barcoded bead library is provided including a plurality of beads with each bead having a set of oligonucleotides attached thereto. Each oligonucleotide within the set includes the same one or more barcodes. The one or more barcodes can be predesigned or can be randomly generated.

In some embodiments, provided herein is a barcoded bead library, wherein the barcode on a bead comprises a capture oligo sequence capable of hybridizing to a capture tag sequence in one or more oligos, e.g., a seed oligo and/or an addition oligo. In some embodiments, the barcode on a bead comprises one or more useful sequences (e.g., other barcode sequences) other than the capture oligo sequence, and one or more useful sequences and the capture oligo sequence may be of the same or different sequences, and/or may be overlapping (e.g., partially overlapping, one within another, or completely overlapping) or non-overlapping.

In some embodiments, in a barcoded bead library, the number of different barcodes (e.g., different capture oligo sequences) on the beads is 2, 3, 4, 5, 6, 7, 8, 9, at least 10, at least 50, at least 100, at least 500, at least 1,000, or any range between the foregoing. In some embodiments, in a barcoded bead library, the number of different barcodes (e.g., different capture oligo sequences) on the beads is from about 2 to about 10, about 10 to about 50, or more than 50. The different barcodes may be provided on the same bead or on two or more beads. In some embodiments, a barcode or a plurality of barcodes define a type of bead among a plurality of different types of beads in the library.

In some embodiments, multiple copies of one or more barcodes are provided on one bead. In some embodiments, the bead comprises 2, 3, 4, 5, 6, 7, 8, 9, at least 10, at least 50, at least 100, at least 500, at least 1,000, at least 10,000, at least 100,000, or at least 1,000,000 copies of one or more barcodes, or any range between the foregoing.

FIG. 1C shows an exemplary pool of addition oligonucleotides in a container such as a vial. The pool may contain one or more sets of addition oligonucleotides, where a set is designed such that the subsequences in the addition oligonucleotides of the set are to be serially assembled (e.g., in a predetermined order) to form a target sequence. Addition oligonucleotides in one set may have the same or different restriction enzyme recognition sequences, compared to addition oligonucleotides in another set. Addition oligonucleotides in one set may have the same or a different adapter, tag, primer binding, cleavage, UMI/UID, and/or barcode sequences, compared to addition oligonucleotides in another set. The oligos including components thereof (e.g., the subsequences, the 3′ overhang sequences, the capture tag sequences, and/or the restriction enzyme (e.g., Type IIS) recognition and cleavage sequences) can be chosen such that the addition oligos are assembled in serial multiplexed reactions, each reaction occurring in parallel with other reactions and in a predetermined order of oligo addition, without interfering with the reaction(s) in a different partition.

FIG. 1D shows, in an exemplary method, that a pool of addition oligos A, B, C, and D are contacted with a library of capture beads, e.g., beads having a capture oligo C1′ or C2′. Capture oligos C1′ and C2′ are capable of hybridizing to capture tag sequences C1 and C2, respectively.

FIG. 1E shows that beads comprising only capture oligo C1′ are capable of capturing hairpin oligos A and B, both of which comprise capture tag sequence C1, while hairpin oligos C and D comprising capture tag sequence C2 are specifically captured on beads comprising only capture oligo C2′. Oligo A and Oligo B comprise subsequences that are to be assembled together to form all or part of a first target sequence, while Oligo C and Oligo D comprise subsequences that are to be assembled together to form all or part of a second target sequence.

FIG. 1F shows, in an exemplary method, that beads with Oligo A and Oligo B captured thereon and beads with Oligo C and Oligo D captured thereon are partitioned into a plurality of partitions, e.g., droplets (e.g., aqueous droplets) within an emulsion, such that on average one or fewer beads occupy the same partition. As such, Oligos A and B are separated from Oligos C and D. The emulsion droplets may comprise one or more other oligos for assembling a target sequence, e.g., one or more seed oligos such as a common oligo (e.g., a universal seed oligo), and/or one or more reagents, such as enzymes, e.g., one or more ligases, one or more polymerases, and/or one or more Type IIS restriction enzymes.

FIG. 1G shows that the captured oligos may be released from the beads, and without breaking the emulsions, a reaction assembling Oligos A and B (and optionally other oligos) into the first target sequence and a reaction assembling Oligos C and D (and optionally other oligos) into the second target sequence may proceed in separate emulsion droplets in parallel and without interfering with each other. After releasing the captured oligos, the beads may remain in or be removed from the partitions. Oligo A is added to the seed oligo first due to sequence complementarity between the seed oligo and Oligo A, and after processing of the assembled polynucleotide (e.g., cleavage by a Type IIS restriction enzyme), Oligo B is then added to the cleaved assembled polynucleotide due to sequence complementarity with Oligo B. Additional oligos may be added to form the first target sequence. A similar reaction occurs in a separate emulsion droplet to assemble the second target sequence comprising subsequences from Oligos C and D. In some examples, only nucleic acid molecules of the same target sequence are assembled in a partition. In other examples, nucleic acid molecules of two or more different target sequences are assembled in a partition. For example, the oligos including components thereof (e.g., the subsequences, the 3′ overhang sequences, the capture tag sequences, and/or the restriction enzyme (e.g., Type IIS) recognition and cleavage sequences) can be chosen such that the addition oligos are assembled in serial multiplexed reactions in the same partition. Each reaction can occur in parallel by adding oligos to a growing assembled product in a predetermined order, without interfering with other reactions in the same partition. The assembled products in the same partition and/or in different partitions may share one or more useful moieties (e.g., sequences), e.g., an adapter, a tag, a primer binding moiety, a cleavage site, a UMI/UID, and/or a barcode. The one or more useful moieties may be provided in a seed oligo, an addition oligo, and/or a terminal oligo.

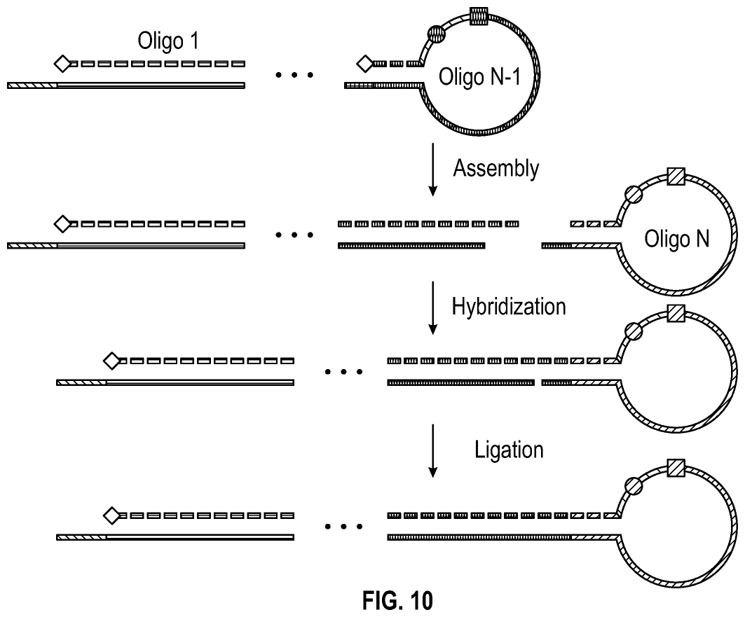

FIG. 1H shows exemplary assembled products after the partitions are combined, for example, by breaking the emulsion to allow droplets to coalesce into a bulk volume and/or by controllably merging two or more droplets. The beads may remain in or be removed from the combined volume. As an example, an assembling reaction may be terminated by the addition of a terminal oligo. For example, the terminal oligo may comprise a hairpin oligo that comprises a 3′ end overhang to hybridize to a 3′ end overhang of an assembled product from the previous cycle, but the assembled product comprising a sequence of the terminal oligo cannot participate in a further cycle of assembly. For instance, the 5′ end of a terminal oligo is not blocked (e.g., dephosphorylated) and, upon hybridization, is ligated to a 3′ end of an assembled product from the previous cycle, such that the 3′ end cannot be extended by a polymerase, e.g., as shown in FIG. 10. In other examples, the terminal oligo may not contain a cleavage site (e.g., a Type IIS recognition and cleavage site), thus the assembled product comprising a sequence of the terminal oligo is not cleaved to provide a sticky end for further addition of oligos. As shown in the figure, the terminal oligo (and/or the seed oligo) may provide one or more useful moieties (e.g., sequences), e.g., an adapter, a tag, a primer binding moiety, a cleavage site, a UMI/UID, and/or a barcode.

FIG. 1I shows exemplary assembled products comprising one or more useful moieties (e.g., sequences) provided by a seed oligo and/or a terminal sequence. The terminal sequence may be provided by any suitable nucleic acid molecules, e.g., single-stranded or double-stranded (e.g., having a blunt end), having one or more free 3′ or 5′ ends, having one or more blocked ends, and/or immobilized or not immobilized on a support. The nucleic acid molecules may but do not have to comprise a hairpin structure (e.g., as shown in FIG. 1H). FIG. 1/also shows that the exemplary assembled products can be amplified, e.g., by using one or more PCR primers that bind to the one or more useful sequences provided by a seed oligo and/or by a terminal oligo. Note a terminal oligo or terminal sequence may be further added to (e.g., by a hairpin oligo or a non-hairpin oligo, both of which may comprise a useful sequence but do not need to comprise a subsequence of a target nucleic acid), and any addition oligo may be designated a terminal oligo depending on the need of assembly, e.g., the need of a first level assembling process and/or a higher level assembling process.

### I. Target Nucleic Acid Sequence and Subsequences Thereof

In some aspects, disclosed herein are methods and compositions for generating a molecule (e.g., linear or circular) comprising a target nucleic acid sequence or a nucleic acid sequence of interest. In some embodiments, the molecule is synthesized or assembled from molecules comprising one or more subsequences (“building blocks”) of one or more target nucleic acid sequences.

The nucleic acids, polynucleotides, and oligonucleotides disclosed herein may comprise naturally-occurring or synthetic polymeric forms of nucleotides. The oligonucleotides and nucleic acid molecules may be formed from naturally occurring nucleotides, for example, forming deoxyribonucleic acid (DNA) or ribonucleic acid (RNA) molecules. Alternatively, the naturally occurring oligonucleotides may include structural modifications to alter their properties, as long as the modified oligonucleotides are compatible with the reactions disclosed herein, e.g., reactions catalyzed by a natural enzyme or an engineered enzyme such as polymerases that have been evolved to amplify a variety of non-natural nucleotides that enable expansion of the genetic code. See, e.g., Houlihan et al., Acc. Chem. Res. 2017, 50, 4, 1079-1087. The present disclosure encompasses equivalents, analogs of either RNA or DNA made from nucleotide analogs and as applicable to the embodiment being described, single-stranded or double-stranded polynucleotides. Nucleotides may include, for example, naturally-occurring nucleotides (for example, ribonucleotides or deoxyribonucleotides), or natural or synthetic modifications of nucleotides, or artificial bases.

In some embodiments, a target nucleic acid sequence is a predetermined sequence or a predefined sequence, such as a sequence that is known or chosen before the synthesis. In some embodiments, a certain degree of randomness in the assembly of one or more subsequences is permitted and encompassed by the present disclosure, and the target nucleic acid sequence or nucleic acid sequence of interest includes such assembled sequences.

Also disclosed herein in some aspects are methods and compositions for generating a plurality of molecules, one of more of which comprise one or more target nucleic acid sequences. In some aspects, disclosed herein are methods for the multiplex synthesis of nucleic acid molecules, in parallel and/or hierarchically, wherein one or more of the nucleic acid molecules comprise one or more target nucleic acid sequences that are known or chosen before the synthesis. In some embodiments, the one or more target nucleic acid sequences may be divided up into a plurality of shorter sequences, e.g., subsequences. In some embodiments, nucleic acid molecules are designed to comprise one or more of the subsequences, and the designed nucleic acid molecules are connected in order to assemble some or all of the subsequences into one or more longer sequences, including eventually the one or more target nucleic acid sequences or any intermediate thereof.

In certain exemplary embodiments, an assembled nucleic acid sequence, including an eventual nucleic acid sequence of interest or target nucleic acid sequence or any intermediate thereof, is at least about 20, 30, 40, 50, 60, 70, 80, 90, 100, 200, 300, 400, 500, 600, 700, 800, 900, 1,000, 1,500, 2,000, 2,500, 3,000, 3,500, 4,000, 4,500, 5,000, 5,500, 6,000, 6,500, 7,000, 7,500, 8,000, 8,500, 9,000, 9,500, 1,000,000, 2,000,000, 3,000,000, 4,000,000, 5,000,000, 6,000,000, 7,000,000, 8,000,000, 9,000,000, 10,000,000 or more nucleotides in length. In other exemplary embodiments, an assembled nucleic acid sequence, including an eventual nucleic acid sequence of interest or target nucleic acid sequence or any intermediate thereof, is between 100 and 10,000,000 nucleic acids in length, including any ranges therein. In yet other exemplary embodiments, an assembled nucleic acid sequence, including an eventual nucleic acid sequence of interest or target nucleic acid sequence or any intermediate thereof, is between 200 and 20,000 nucleic acids in length, including any ranges therein. In still other exemplary embodiments, an assembled nucleic acid sequence, including an eventual nucleic acid sequence of interest or target nucleic acid sequence or any intermediate thereof, is between 500 and 25,000 nucleic acids in length, including any ranges therein. In still other exemplary embodiments, an assembled nucleic acid sequence, including an eventual nucleic acid sequence of interest or target nucleic acid sequence or any intermediate thereof, is between 300 and 5,000 nucleic acids in length, including any ranges therein. In still other exemplary embodiments, an assembled nucleic acid sequence, including an eventual nucleic acid sequence of interest or target nucleic acid sequence or any intermediate thereof, is between 1,000 and 100,000 nucleic acids in length, including any ranges therein.

In certain exemplary embodiments, an assembled nucleic acid sequence, including an eventual nucleic acid sequence of interest or target nucleic acid sequence or any intermediate thereof, is of the length of a gene, e.g., between about 500 nucleotides and 5,000 nucleotides in length, or a fragment thereof. In other aspects, an assembled nucleic acid sequence, including an eventual nucleic acid sequence of interest or target nucleic acid sequence or any intermediate thereof, is of the length of a chromosome (e.g., a phage chromosome, a viral chromosome, a bacterial chromosome, a fungal (e.g., yeast) chromosome, an organelle chromosome (e.g., a mitochondrial chromosome), a plant chromosome, an animal chromosome or the like) or a fragment thereof. In still other aspects, an assembled nucleic acid sequence, including an eventual nucleic acid sequence of interest or target nucleic acid sequence or any intermediate thereof, is the length of a genome (e.g., a phage genome, a viral genome, a bacterial genome, a fungal (e.g., yeast) genome, a plant genome, an animal genome or the like) or a fragment thereof.

In certain exemplary embodiments, an assembled nucleic acid sequence, including an eventual nucleic acid sequence of interest or target nucleic acid sequence or any intermediate thereof, comprises a DNA sequence. In other embodiments, an assembled nucleic acid sequence, including an eventual nucleic acid sequence of interest or target nucleic acid sequence or any intermediate thereof, comprises an RNA sequence, such as an mRNA sequence that can be translated in vitro or in vivo (e.g., to produce a polypeptide), or a regulatory RNA sequence such as lincRNA (long intergenic non-coding RNA) or lncRNA (long non-coding RNA).

In certain exemplary embodiments, an assembled nucleic acid sequence, including an eventual nucleic acid sequence of interest or target nucleic acid sequence or any intermediate thereof, comprises a sequence such as a regulatory element (e.g., a promoter region, an enhancer region, a coding region, a non-coding region and the like), a gene, a gene cluster, an extrachromosomal nucleic acid sequence such as an extrachromosomal DNA, a nucleic acid in an organelle, such as a nucleic acid in a mitochondria (e.g., mitochondrial DNA) or plastid (e.g., a chloroplast), a chromosome or fragment thereof, or a genome, e.g., of or derived from a viral, bacterial, fungal (e.g. yeast), or other prokaryotic or eukaryotic (e.g., mammalian) organism. In certain exemplary embodiments, an assembled nucleic acid sequence, including an eventual nucleic acid sequence of interest or target nucleic acid sequence or any intermediate thereof, comprises a sequence of or derived from a viral, bacterial, fungal (e.g. yeast), or other prokaryotic or eukaryotic (e.g., mammalian) organism.

In certain exemplary embodiments, one or more assembled nucleic acid sequences, including an eventual nucleic acid sequence of interest or target nucleic acid sequence or any intermediate thereof, comprise one or more sequences that are contiguous in a natural context, e.g., a contiguous sequence in a native gene locus, native gene cluster, native chromosome or fragment thereof (including coding and/or noncoding sequences), or native genome. In certain exemplary embodiments, one or more assembled nucleic acid sequences, including an eventual nucleic acid sequence of interest or target nucleic acid sequence or any intermediate thereof, comprise sequences that are not contiguous in a natural context. For instance, sequences from discrete locations in a native gene locus, native gene cluster, native chromosome or fragment thereof (including coding and/or noncoding sequences), or native genome may be artificially assembled in one or more assembled nucleic acid sequences.

In certain exemplary embodiments, one or more assembled nucleic acid sequences, including an eventual nucleic acid sequence of interest or target nucleic acid sequence or any intermediate thereof, comprise one or more sequences that form a genome, proteome, and/or RNAome (e.g., transcriptome), or any subset thereof, e.g., a kinome; a secretome; a receptome (e.g., GPCRome); an immunoproteome; a nutriproteome; a proteome subset defined by a post-translational modification (e.g., phosphorylation, ubiquitination, methylation, acetylation, glycosylation, oxidation, lipidation, and/or nitrosylation), such as a phosphoproteome (e.g., phosphotyrosine-proteome, tyrosine-kinome, and tyrosine-phosphatome), a glycoproteome, etc.; a proteome subset associated with a tissue or organ, a developmental stage, or a physiological or pathological condition; a proteome subset associated a cellular process, such as cell cycle, differentiation (or de-differentiation), cell death, senescence, cell migration, transformation, or metastasis; or any subset thereof, or any combination thereof; transcriptome; miRNAome, or a subset thereof. In certain exemplary embodiments, one or more assembled nucleic acid sequences, including an eventual nucleic acid sequence of interest or target nucleic acid sequence or any intermediate thereof, comprise one or more sequences that form a pathway (e.g., a metabolic pathway (e.g., nucleotide metabolism, carbohydrate metabolism, amino acid metabolism, lipid metabolism, co-factor metabolism, vitamin metabolism, energy metabolism and the like), a signaling pathway, a biosynthetic pathway, an immunological pathway, a developmental pathway and the like) and the like. In some embodiments, one or more assembled nucleic acid sequences, including an eventual nucleic acid sequence of interest or target nucleic acid sequence or any intermediate thereof, comprise one or more sequences of a genome with an altered genetic code. For example, genes can be re-coded to use only a subset of possible codons, and the newly freed-up codons can be re-purposed to incorporate additional (e.g., unnatural) amino acids. In such example, tRNAs and associated machinery (aminoacyl tRNA synthetases) can adapted to produce tRNAs charged with the new amino acids. In some embodiments, recoding with removal of the tRNAs for the cognate codons can protect an organism from pathogens that require host machinery to translate their genes.

In certain exemplary embodiments, one or more assembled nucleic acid sequences, including an eventual nucleic acid sequence of interest or target nucleic acid sequence or any intermediate thereof, comprise one or more sequences that are difficult to synthesize, difficult to amplify, and/or difficult to sequence verify. In some embodiments, one or more assembled nucleic acid sequences, including an eventual nucleic acid sequence of interest or target nucleic acid sequence or any intermediate thereof, comprise a sequence difficult to synthesize using an approach comprising base-by-base nucleic acid synthesis. In some embodiments, one or more assembled nucleic acid sequences, including an eventual nucleic acid sequence of interest or target nucleic acid sequence or any intermediate thereof, comprise a homopolymer sequence, e.g., Aₙ; a homocopolymer sequence, e.g., [AT]ₙ; a sequence comprising direct repeats; an AT-rich sequence; a GC-rich sequence, or any combination thereof. In some embodiments, one or more assembled nucleic acid sequences comprises a sequence that is prone to mis-hybridize (e.g., GC-rich sequences or repetitive sequences), e.g., a linear oligo comprising the sequence used for hybridization during the assembly may hybridize in the wrong order and/or to incorrect locations. In some embodiments, the methods and compositions disclosed herein are used to assemble long sequences, and the sequence prone to mis-hybridize is kept double-stranded in a growing chain, avoiding potential mis-hybridization problems caused by the sequence prone to mis-hybridize.

In some embodiments, one or more sequences that are difficult to synthesize, difficult to amplify, and/or difficult to sequence verify may be included in an oligo disclosed herein, for example, in the loop region of a hairpin oligo.

In some embodiments, the plurality of shorter sequences, e.g., subsequences, comprise one or more sequences that are difficult to synthesize, difficult to amplify, and/or difficult to sequence verify. In some embodiments, a long sequence is assembled from a plurality of shorter sequences, wherein one or more of the shorter sequences are easier to synthesize than the long sequence. For instance, a long sequence comprising repeats may be assembled from a plurality of shorter sequences comprising repeats, wherein one or more of the shorter repeat sequences are easier to synthesize than the long repeat sequence.

In some embodiments, the plurality of shorter sequences, e.g., subsequences, are non-overlapping sequences within a target nucleic acid sequence. In other embodiments, two or more of the plurality of shorter sequences, e.g., subsequences, are at least partially overlapping sequences within a target nucleic acid sequence. In any of the embodiments herein, all or a subset of the plurality of shorter sequences, e.g., subsequences, can be assembled to form the target nucleic acid sequence. In some embodiments, for example in the case of partially overlapping subsequences, the overlapping sequence or sequences are not duplicated in the assembled sequence, including the eventual target nucleic acid sequence or any intermediate thereof.

In some embodiments, one or more of the plurality of shorter sequences, e.g., subsequences, are from 10 to about 300 nucleotides, from 20 to about 400 nucleotides, from 30 to about 500 nucleotides, from 40 to about 600 nucleotides, or more than about 600 nucleotides long. In some embodiments, the plurality of shorter sequences, e.g., subsequences, are between about 10 and about 20, about 20 and about 30, about 30 and about 40, about 40 and about 50, about 50 and about 60, about 60 and about 70, about 70 and about 80, about 80 and about 90, about 90 and about 100, about 100 and about 110, about 110 and about 120, about 120 and about 130, about 130 and about 140, about 140 and about 150, about 150 and about 160, about 160 and about 170, about 170 and about 180, about 180 and about 190, about 190 and about 200, about 200 and about 210, about 210 and about 220, about 220 and about 230, about 230 and about 240, about 240 and about 250, about 250 and about 260, about 260 and about 270, about 270 and about 280, about 280 and about 290, about 290 and about 300, or more than about 300 nucleotides in length.

In some embodiments, one or more of the plurality of shorter sequences, e.g., subsequences, are between about 100 and about 200, about 200 and about 300, about 300 and about 400, about 400 and about 500, about 500 and about 600, about 600 and about 700, about 700 and about 800, about 800 and about 900, about 900 and about 1,000, or more than about 1,000 nucleotides long. In some embodiments, one or more of the plurality of shorter sequences, e.g., subsequences, are between about 1,000 and about 2,000, about 2,000 and about 3,000, about 3,000 and about 4,000, about 4,000 and about 5,000, about 5,000 and about 6,000, or more than about 6,000 nucleotides long.

In some embodiments, the average length of the plurality of shorter sequences, e.g., subsequences, is from 10 to about 300 nucleotides, from 20 to about 400 nucleotides, from 30 to about 500 nucleotides, from 40 to about 600 nucleotides, or more than about 600 nucleotides long. In some embodiments, the average length of the plurality of shorter sequences, e.g., subsequences, is between about 10 and about 20, about 20 and about 30, about 30 and about 40, about 40 and about 50, about 50 and about 60, about 60 and about 70, about 70 and about 80, about 80 and about 90, about 90 and about 100, about 100 and about 110, about 110 and about 120, about 120 and about 130, about 130 and about 140, about 140 and about 150, about 150 and about 160, about 160 and about 170, about 170 and about 180, about 180 and about 190, about 190 and about 200, about 200 and about 210, about 210 and about 220, about 220 and about 230, about 230 and about 240, about 240 and about 250, about 250 and about 260, about 260 and about 270, about 270 and about 280, about 280 and about 290, about 290 and about 300, or more than about 300 nucleotides in length.

In some embodiments, the average length of the plurality of shorter sequences, e.g., subsequences, is between about 100 and about 200, about 200 and about 300, about 300 and about 400, about 400 and about 500, about 500 and about 600, about 600 and about 700, about 700 and about 800, about 800 and about 900, about 900 and about 1,000, or more than about 1,000 nucleotides long. In some embodiments, the average length of the plurality of shorter sequences, e.g., subsequences, is between about 1,000 and about 2,000, about 2,000 and about 3,000, about 3,000 and about 4,000, about 4,000 and about 5,000, about 5,000 and about 6,000, or more than about 6,000 nucleotides long.

In some embodiments, the plurality of shorter sequences, e.g., subsequences, have the same length. In some embodiments, at least one of the plurality of shorter sequences, e.g., subsequences, has a different length from at least one other of the plurality of shorter sequences. In some embodiments, the plurality of shorter sequences, e.g., subsequences, have substantially the same length. In some embodiments, at least 50%, at least 60%, at least 70%, at least 80%, at least 90%, at least 95%, or at least 99% of the plurality of shorter sequences, e.g., subsequences, have the same length. In some embodiments, at least 50%, at least 60%, at least 70%, at least 80%, at least 90%, at least 95%, at least 99%, or all of the plurality of shorter sequences, e.g., subsequences, are within ±50% of a target length, ±40% of a target length, ±30% of a target length, ±20% of a target length, ±10% of a target length, ±5% of a target length, ±1% of a target length, or of a target length. In some embodiments, the target length is between about 100 and about 200, about 200 and about 300, about 300 and about 400, about 400 and about 500, about 500 and about 600, about 600 and about 700, about 700 and about 800, about 800 and about 900, about 900 and about 1,000, or more than about 1,000 nucleotides long. In some embodiments, the average length of the plurality of shorter sequences, e.g., subsequences, is between about 1,000 and about 2,000, about 2,000 and about 3,000, about 3,000 and about 4,000, about 4,000 and about 5,000, about 5,000 and about 6,000, or more than about 6,000 nucleotides long.

### II. Nucleic Acid Molecules Comprising Subsequences

In some aspects, provided herein are a plurality of nucleic acid molecules designed to comprise one or more subsequences which are to be assembled (with one or more subsequences in one or more other nucleic acid molecules of the plurality of nucleic acid molecules, and/or with one or more sequences other than those in the plurality of nucleic acid molecules) to form one or more assembled nucleic acid sequences, including one or more nucleic acid sequences of interest or target nucleic acid sequences or any intermediate thereof. In other aspects, provided herein are methods comprising designing and/or obtaining the plurality of nucleic acid molecules. The solid phase synthesis of oligonucleotides and nucleic acid molecules with naturally occurring or artificial bases is well known in the art.

In various embodiments, the methods described herein use oligonucleotides, their sequence being determined based on the sequence of the final polynucleotide constructs to be synthesized. In one embodiment, oligonucleotides are short nucleic acid molecules. For example, oligonucleotides may be from 10 to about 300 nucleotides, from 20 to about 400 nucleotides, from 30 to about 500 nucleotides, from 40 to about 600 nucleotides, or more than about 600 nucleotides long. However, shorter or longer oligonucleotides may be used. Oligonucleotides may be designed to have different lengths.

The oligonucleotides according to the present disclosure which are used to assemble or create an assembled nucleic acid sequence can be synthesized using standard column-synthesized techniques or on DNA microchips. For any individual assembly of a target nucleic acid, the oligonucleotides within the set of oligonucleotides may contain the same barcode sequence, orthogonal or otherwise. The oligonucleotides may then be annealed to an orthogonal bead library. According to this aspect, each bead includes all or a subset of oligonucleotides which are used to create a target nucleic acid sequence.

In some embodiments, the collection of barcode sequences within the set of oligonucleotides is chosen (e.g., designed and/or selected) to have similar hybridization melting temperatures, so that capture on beads can be carried out under relatively uniform conditions. For example, in an emulsion, all or a majority of the droplets can be maintained at the same temperature, or have the same temperature profile if it is changed. In some embodiments, the barcode sequences are sufficiently unique to avoid or reduce cross-hybridization and/or non-specific hybridization.

In some embodiments, immobilized oligonucleotides or polynucleotides are used as a source of material to generate the “building block” oligos disclosed herein. Oligonucleotides can be synthesized using methods known to those of skill in the art and described herein such as column-synthesis or chip synthesis or taken directly from a prefabricated chip and pooled. According to one aspect, oligonucleotides or polynucleotides libraries may but need not be amplified to create useful oligonucleotides for use in the methods described herein. For example, oligonucleotides can be obtained from microarrays or chips or synthesized for use in the methods described herein.

In some aspects, the oligonucleotides can be amplified before being processed into a library using methods known to those of skill in the art and described herein. According to one aspect, the oligonucleotides can be single stranded or double stranded. Double stranded oligonucleotides can be rendered single stranded using methods known to those of skill in the art and described herein. The oligonucleotides can include a barcode or primer. The barcode or primer can be included in the original synthesis of the oligonucleotide or it can be added to a fully formed oligonucleotide.

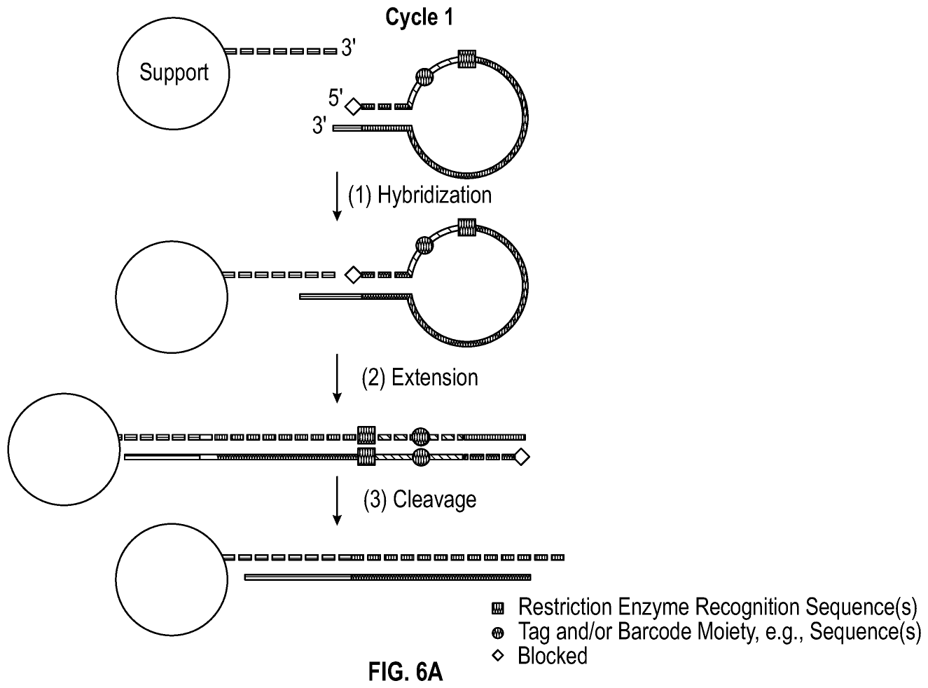

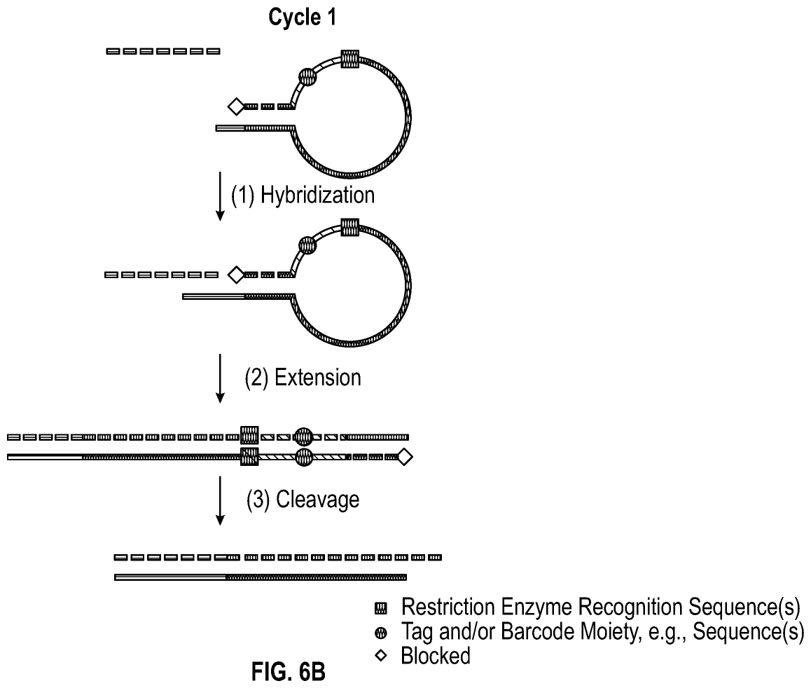

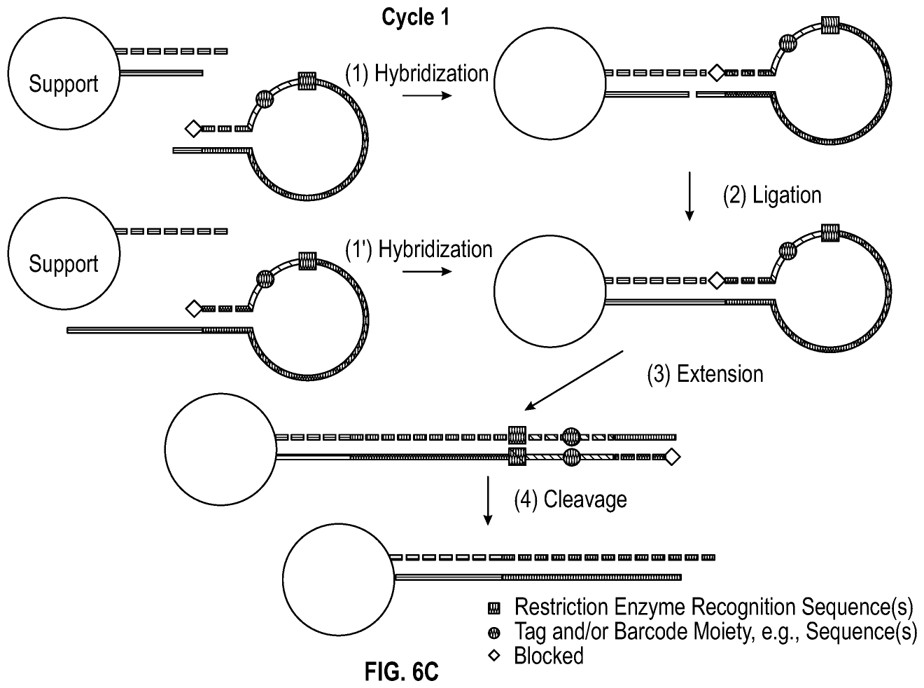

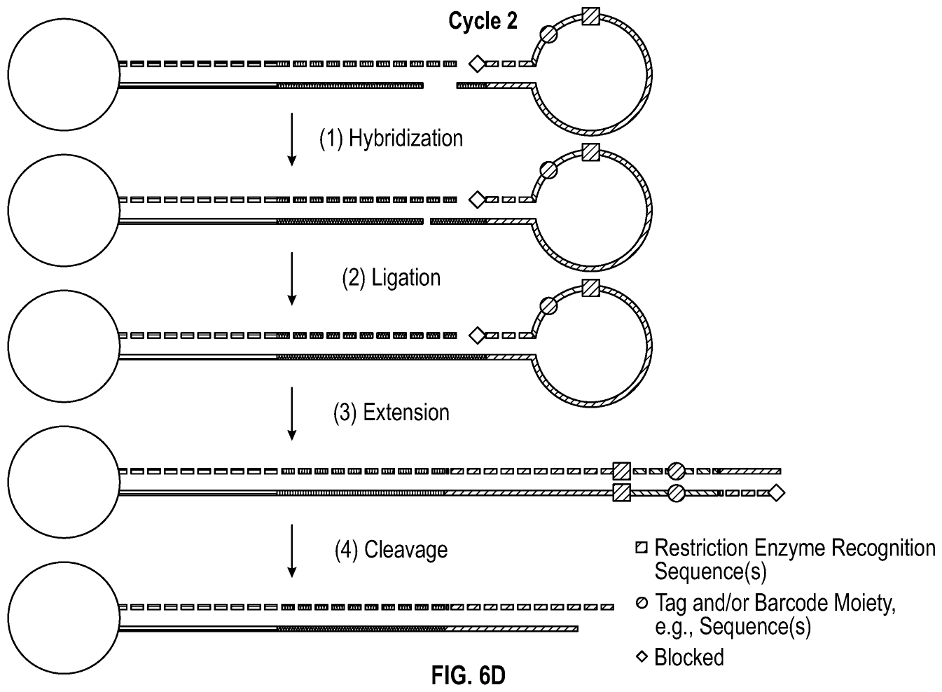

In some aspects, for example, barcodes and/or primers (and/or any other one or more useful sequences disclosed herein, e.g., in Section II-B-d) can be detached from the oligonucleotide using methods known to those of skill in the art and described herein, for example, a restriction enzyme recognition site can be present within the oligonucleotide, and a restriction enzyme can be used to cleave the oligonucleotide at or near the restriction enzyme recognition site thereby separating a barcode or primer from the remaining oligonucleotide sequence. Other methods and materials known to those of skill in the art can also be used to separate a barcode or primer from the remaining oligonucleotide sequence such as a USER enzyme. In certain embodiments, the one or more useful sequences are removed from an assembled product during the concerted sequential addition of oligos, e.g., as shown in FIGS. 6-9. In certain embodiments, the one or more useful sequences are removed from an assembled product after the completion of a sequential additions of oligos, e.g., to prepare an assembled product for a higher level assembly or for a downstream analysis or application (e.g., for transfecting or transforming a cell).

The polynucleotides disclosed herein may comprise one or more deoxyribonucleotides, ribonucleotides, modified nucleotides, and/or modified nucleosides, such as methylated nucleotides and nucleotide analogs, uracyl, other sugars, and linking groups such as fluororibose and thioate, and nucleotide branches. In some embodiments, the polynucleotides disclosed herein may include non-nucleotide components. Exemplary modified nucleic acids include amine-modified nucleotides such as aminoallyl (aa)-dUTP, aa-dCTP, aa-dGTP, and/or aa-dATP, 2-Aminopurine, 2,6-Diaminopurine (2-Amino-dA), inverted dT, 5-Methyl dC, 2′-deoxy-Inosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), and combinations of the foregoing.

In certain embodiments, methods are provided for designing a set of oligonucleotides for each nucleic acid sequence of interest, e.g., a gene, a regulatory element, a vector, a construct, a chromosome (e.g., an artificial chromosome), a genome (e.g., an artificial genome), or the like. In another aspect, oligonucleotide design is aided by a computer program.

A. Seed Nucleic Acid Molecule

In some embodiments, provided herein is a seed nucleic acid molecule, which in some instances is also referred to as a nucleating nucleic acid molecule, especially when additional nucleic acid molecules are added to more than one end of the nucleic acid molecule. In some embodiments, the seed nucleic acid molecule is a seed oligonucleotide (“seed oligo”). In some embodiments, a seed nucleic acid molecule comprises one or more subsequences of a target nucleic acid sequence. In some embodiments, a seed nucleic acid molecule does not comprise a subsequence of a target nucleic acid sequence, and an addition nucleic acid molecule to be added to the nucleic acid molecule comprises one or more subsequences of a target nucleic acid sequence.

In some embodiments, provided herein are a plurality of seed nucleic acid molecules, e.g., seed oligos. In some embodiments, some or all of the plurality of seed nucleic acid molecules are the same, e.g., as a universal seed nucleic acid molecule for the assembly of two or more assembled sequences having at least a difference in sequence and/or length. In some embodiments, some or all of the plurality of seed nucleic acid molecules comprise the same subsequence or subsequences. In some embodiments, some or all of the plurality of seed nucleic acid molecules have at least a difference in sequence and/or length. In some embodiments, some or all of the plurality of seed nucleic acid molecules comprise subsequences that have at least a difference in length, sequence, and/or nucleic acid backbone and/or base modification.

In some embodiments, the seed nucleic acid molecule comprises one or more 3′ end sequences of one or more nucleotides in length capable of hybridizing to a 3′ end sequence of one or more nucleotides in length of another nucleic acid molecule, e.g., a nucleic acid molecule (such as a hairpin oligo comprising a subsequence of a target nucleic acid sequence) to be added to the seed nucleic acid molecule.

In some embodiments, the seed nucleic acid molecule is a single-stranded polynucleotide, e.g., a single-stranded oligo comprising a 3′ end sequence capable of hybridizing to a 3′ end sequence of an addition nucleotide acid molecule such as a hairpin addition oligo, e.g., as disclosed in Section II-B. In some embodiments, the single-stranded seed polynucleotide does not comprise a subsequence of a target nucleic acid sequence or intermediate thereof to be assembled. For example, the single-stranded seed polynucleotide comprises one or more sequences useful for assembling the target nucleic acid sequence or intermediate thereof and/or the subsequent detection, analysis, and/or use of the assembled sequence, but the one or more useful sequences may be removed and do not need to be present in the assembled target nucleic acid sequence or intermediate thereof. For example, the single-stranded seed polynucleotide may comprise any one or more of an adapter moiety (e.g., an adapter sequence such as a universal adapter sequence and/or an adapter for sequencing, such as P5 or P7), a tag moiety (e.g., a tag sequence and/or an affinity tag, for hybridization or affinity-based capture onto a support), a primer binding sequence, an amplification sequence, a cleavage site or sequence (e.g., a restriction enzyme recognition sequence and cleavage site), a unique molecular identifier (UMI), a unique identifier (UID), a primer ID, and a barcode, any one or more of which may be unique to the seed polynucleotide or to a subset of seed polynucleotides among a plurality of seed polynucleotides. In some embodiments, the single-stranded seed polynucleotide comprises a subsequence of a target nucleic acid sequence, e.g., a subsequence in the plus or minus strand of a double-stranded target nucleic acid, where a portion or all of the subsequence in the seed polynucleotide is present in the assembled target nucleic acid sequence or intermediate thereof. In some embodiments, in addition to the subsequence, the single-stranded seed polynucleotide comprises any one or more of an adapter moiety, a tag moiety, a primer binding sequence, an amplification sequence, a cleavage site or sequence, a unique molecular identifier (UMI), a unique identifier (UID), a primer ID, and a barcode, any one of which may have a sequence that is the same as or distinct from the subsequence, and/or any one of which may be non-overlapping or partially or completely overlapping with the subsequence.

The seed nucleic acid molecule can be of any suitable length and/or composition (e.g., nucleic acid backbone and/or base compositions including modifications), e.g., as long as the seed oligo comprises a 3′ end sequence capable of hybridizing to a 3′ end sequence of an addition nucleotide acid molecule such as a hairpin addition oligo, e.g., as disclosed in Section II-B, where the 3′ end sequence of the seed oligo is capable of serving as a primer for extension by a polymerase by using all or part of the addition nucleotide acid molecule as template. In some embodiments, the seed nucleic acid molecule is between about 2 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 65 and about 70, about 70 and about 75, about 75 and about 80, about 80 and about 85, about 85 and about 90, about 90 and about 95, about 95 and about 100, or more than about 100 nucleotides in length.

In some embodiments, the seed nucleic acid molecule comprises two, three, four, or more than four strands, e.g., as a duplex comprising a 3′ end sequence (e.g., a 3′ overhang) capable of hybridizing to a 3′ end sequence of an addition nucleotide acid molecule such as a hairpin addition oligo, e.g., as disclosed in Section II-B. In some embodiments, the seed nucleic acid molecule comprises one, two, three, four, or more than four 3′ overhangs, one or more of which is capable of hybridizing to a 3′ end sequence of an addition nucleotide acid molecule. In some embodiments, the seed polynucleotide does not comprise a subsequence of a target nucleic acid sequence or intermediate thereof to be assembled. For example, the seed polynucleotide can comprise one or more sequences useful for assembling the target nucleic acid sequence or intermediate thereof and/or the subsequent detection, analysis, and/or use of the assembled sequence, but the one or more useful sequences may be removed and do not need to be present in the assembled target nucleic acid sequence or intermediate thereof. For example, the seed polynucleotide may comprise any one or more of an adapter moiety (e.g., an adapter sequence such as a universal adapter sequence and/or an adapter for sequencing, such as P5 or P7), a tag moiety (e.g., a tag sequence and/or an affinity tag, for hybridization or affinity-based capture onto a support), a primer binding sequence, an amplification sequence, a cleavage site or sequence (e.g., a restriction enzyme recognition sequence and cleavage site), a unique molecular identifier (UMI), a unique identifier (UID), a primer ID, and a barcode, any one or more of which may be unique to the seed polynucleotide or to a subset of seed polynucleotides among a plurality of seed polynucleotides. In some embodiments, the seed polynucleotide comprises a subsequence of a target nucleic acid sequence, e.g., a subsequence in the plus or minus strand of a double-stranded target nucleic acid, where a portion or all of the subsequence in the seed polynucleotide is present in the assembled target nucleic acid sequence or intermediate thereof. The subsequence may be present in a double-stranded and/or a single-stranded region of the seed polynucleotide. In some embodiments, in addition to the subsequence, the seed polynucleotide comprises any one or more of an adapter moiety, a tag moiety, a primer binding sequence, an amplification sequence, a cleavage site or sequence, a unique molecular identifier (UMI), a unique identifier (UID), a primer ID, and a barcode, any one of which may have a sequence that is the same as or distinct from the subsequence, and/or any one of which may be non-overlapping or partially or completely overlapping with the subsequence.

The seed nucleic acid molecule can be of any suitable length and/or composition (e.g., nucleic acid backbone and/or base compositions including modifications), e.g., as long as the seed oligo comprises a 3′ end sequence (e.g., a 3′ overhang) capable of hybridizing to a 3′ end sequence of an addition nucleotide acid molecule such as a hairpin addition oligo, e.g., as disclosed in Section II-B, where the 3′ end sequence of the seed oligo is capable of serving as a primer for extension by a polymerase by using all or part of the addition nucleotide acid molecule as template. In some embodiments, a duplex region of the seed nucleic acid molecule is between about 2 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 65 and about 70, about 70 and about 75, about 75 and about 80, about 80 and about 85, about 85 and about 90, about 90 and about 95, about 95 and about 100, or more than about 100 base pairs in length. In some embodiments, a 3′ overhang of the seed nucleic acid molecule is 1, 2, 3, 4, 5, 6, 7, 8, 9, 10, 11, 12, 13, 14, 15, 16, 17, 18, 19, 20, or between about 20 and about 25, about 25 and about 30, about 30 and about 35, about 35 and about 40, about 40 and about 45, about 45 and about 50, about 50 and about 55, about 55 and about 60, about 60 and about 65, about 65 and about 70, about 70 and about 75, about 75 and about 80, about 80 and about 85, about 85 and about 90, about 90 and about 95, about 95 and about 100, or more than about 100 nucleotides in length.

In some embodiments, all or part of the seed nucleic acid molecule forms a duplex. In some embodiments, all or part of the seed nucleic acid molecule forms one or more stem-loop structures. In some embodiments, the seed nucleic acid molecule comprises a single-stranded region and a double-stranded region. In some embodiments, the seed nucleic acid molecule comprises a sticky end (also referred to a cohesive end), e.g., a 3′ sequence that does not hybridize or is not complementary to any other sequence in the seed nucleic acid molecule. In some embodiments, the seed nucleic acid molecule comprises a 3′ unhybridized sequence. In some embodiments, the seed nucleic acid molecule comprises a 3′ overhang. In some embodiments, the seed nucleic acid molecule comprises two sticky ends, e.g., two 3′ sequences that do not hybridize or are not complementary to any other sequence in the seed nucleic acid molecule. In some embodiments, the seed nucleic acid molecule comprises two 3′ unhybridized sequences. In some embodiments, the seed nucleic acid molecule comprises two 3′ overhangs. In some embodiments, the seed nucleic acid molecule comprises one or more 5′ sequences that hybridize or are complementary to a sequence in the seed nucleic acid molecule. In some embodiments, the seed nucleic acid molecule comprises one or more 5′ sequences that do not hybridize or are not complementary to any other sequence in the seed nucleic acid molecule.

In some embodiments, the seed nucleic acid molecule is attached covalently or non-covalently to a support, e.g., immobilized on a bead. For example, one or more seed nucleic acid molecules may be provided on a plurality of beads which are partitioned into a plurality of reaction volumes, e.g., emulsion droplets containing a bead, for example, for parallel assembly of one or more seed nucleic acid molecules and one or more addition nucleic acid molecules in the plurality of reaction volumes. In some embodiments, the one or more seed nucleic acid molecules on the beads comprise a universal or comment sequence for the reactions in all or a subset of the plurality of reaction volumes. In some embodiments, the one or more seed nucleic acid molecules on the beads are universal or common for the reactions in all or a subset of the plurality of reaction volumes.

In some embodiments, the seed nucleic acid molecule is not attached to a support, e.g., a bead, and is in a soluble form. For example, one or more seed nucleic acid molecules may be provided in a bulk solution which is partitioned into a plurality of reaction volumes, e.g., emulsion droplets containing a bead, for example, for parallel assembly of one or more seed nucleic acid molecules and one or more addition nucleic acid molecules in the plurality of reaction volumes. In some embodiments, the one or more seed nucleic acid molecules comprise a universal or comment sequence for the reactions in all or a subset of the plurality of reaction volumes. In some embodiments, the one or more seed nucleic acid molecules are universal or comment for the reactions in all or a subset of the plurality of reaction volumes.

In some embodiments, the seed nucleic acid molecule comprises a blocked end, e.g., an end blocked from ligation (e.g., by an ligase or chemical ligation) and/or primer extension by a polymerase. In some embodiments, the seed nucleic acid molecule does not comprise a blocked end.

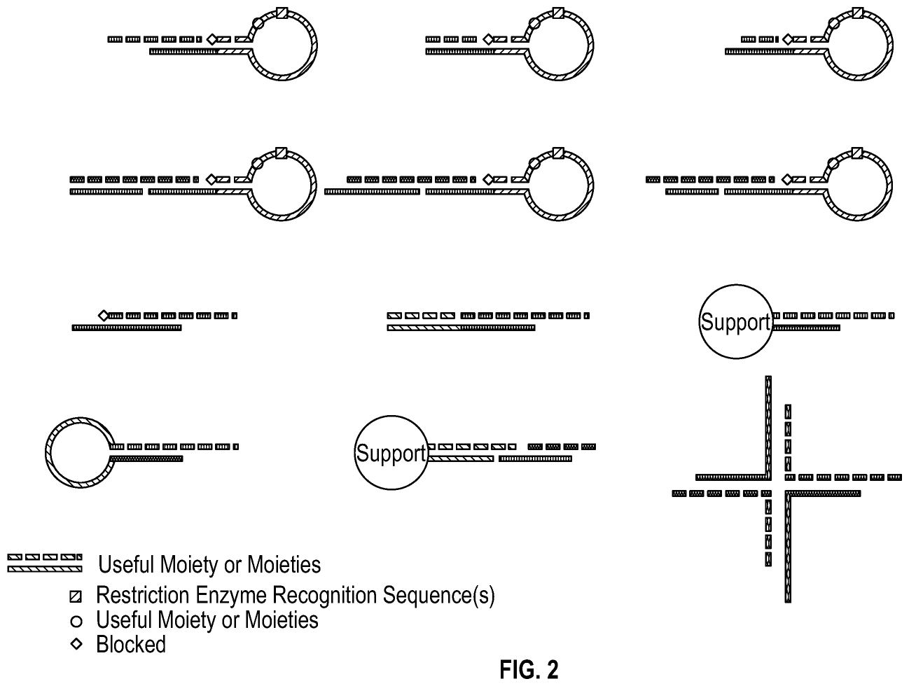

Exemplary seed nucleic acid molecules are shown in FIG. 2. For instance, a seed nucleic acid molecule can be a single-stranded oligo that comprises a 3′ end sequence capable of hybridizing to a 3′ end overhang of a hairpin addition oligo. In some examples, only a portion of seed nucleic acid molecule hybridizes to the 3′ end overhang, leaving a 5′ end overhang in the hybridization complex. In some examples, the entire sequence of the seed nucleic acid molecule hybridizes to the 3′ end overhang, forming a blunt end or a 3′ end overhang in the hybridization complex. In some examples, a seed nucleic acid molecule is a double-stranded oligo that comprises a 3′ end sequence capable of hybridizing to a 3′ end overhang of a hairpin addition oligo. Upon hybridization, the complex may comprise a blunt end, a 3′ end overhang, or a 5′ end overhang.

As shown in FIG. 2, an exemplary seed nucleic acid molecule may also comprise one or more adapter, tag, primer binding, cleavage, UMI, and/or barcode moieties. The seed nucleic acid molecule may also be attached to a support, such as a bead or substrate (e.g., a planar substrate), and/or comprise one or more loops, such as those in hairpin or stem-loop structures. In some embodiments, the seed nucleic acid molecule may comprise one or more structures disclosed herein in any suitable combination and/or in any suitable arrangement (e.g., order of the one or more structures) in the molecule. For example, the seed nucleic acid molecule may comprises a duplex, one end of which comprises a 3′ overhang whereas the other 3′ end overhang is capable of hybridizing to an adapter, tag, primer binding, cleavage, UMI, and/or barcode sequence that is covalently or non-covalently attached to a support (e.g., bead or solid substrate). In some embodiments, the seed nucleic acid molecule comprises one or two sticky ends, e.g., 3′ overhangs. In some embodiments, the seed nucleic acid molecule comprises more than two sticky ends, e.g., 3′ overhangs, such as the molecule formed by four strands shown in FIG. 2.

B. Addition Nucleic Acid Molecule

In some embodiments, provided herein is an addition nucleic acid molecule, which can be used as a building block during the assembly of a plurality of subsequences into a target nucleic acid sequence. In some embodiments, the addition nucleic acid molecule is an addition oligonucleotide (“addition oligo”).

In some embodiments, provided herein is an addition nucleic acid molecule comprising, in the 3′ to 5′ direction: (i) a single-stranded 3′ end sequence, (ii) a subsequence of a target nucleic acid sequence, (iii) a cleavage enzyme recognition sequence such as a Type IIS restriction enzyme recognition sequence, and (iv) a complementary sequence capable of hybridizing to all or a portion of the subsequence.

In some embodiments, the addition nucleic acid molecule is a single-strand molecule capable of forming a hairpin structure. In some embodiments, the hairpin molecule comprises a 3′ single-stranded region that does not hybridize to another sequence of the addition nucleic acid molecule, e.g., the hairpin molecule comprises a 3′ overhang. In some embodiments, the hairpin molecule further comprises a duplex stem region formed by intramolecular nucleotide base pairing between all or a portion of the subsequence and the complementary sequence. In some embodiments, the hairpin molecule further comprises a loop region. In some embodiments, the addition nucleic acid molecule is in a configuration that is not cleaved or not cleavable by the cleavage enzyme. In some embodiments, the addition nucleic acid molecule is in a configuration that is not cleaved or not cleavable by the Type IIS restriction enzyme. All or a portion of the restriction enzyme recognition sequence and/or its cleavage sequence may be in a substantially single-stranded region of the hairpin molecule, such as in the loop region. For instance, the restriction enzyme recognition sequence and its cleavage sequence are in a substantially single-stranded region of the hairpin molecule, such that before the hairpin loop is converted into a duplex (e.g., using primer extension by a polymerase using the single-stranded region as a template), the restriction enzyme does not recognize the single-stranded recognition sequence and/or does not cleave the hairpin molecule. In some embodiments, all or a portion of the restriction enzyme recognition sequence is in a single-stranded region of the hairpin molecule. In some embodiments, all or a portion of the restriction enzyme cleavage site is in a single-stranded region of the hairpin molecule.

In some embodiments, provided herein are a plurality of addition nucleic acid molecules, e.g., addition oligos. In some embodiments, the plurality of addition nucleic acid molecules comprise sets P11, . . . , and P1j₁; . . . ; Pk1, . . . , and Pkjₖ; . . . ; and Pi1, . . . , and Pijᵢ, wherein i, j₁, . . . , jₖ, . . . , jᵢ, and k are integers, i, j₁, . . . , jₖ, . . . , and jᵢ are independently 2 or greater, and 1≤k≤i. In some embodiments, Pk1, . . . , and Pkjₖ comprise subsequences Sk1, . . . , and Skjₖ, respectively, which form target sequence S′k. Thus, sets P11, . . . , and P1jᵢ; . . . ; Pk1, . . . , and Pkjₖ; . . . ; and Pi1, . . . , and Pijᵢ can be used for assembling target sequences S′1, . . . , S′k, . . . , and S′i, respectively. In some embodiments, some or all of sets P11, . . . , and P1j₁; . . . ; Pk1, . . . , and Pkjₖ; . . . ; and Pi1, . . . , and Pijᵢ share one or more addition nucleic acid molecules. For example, some or all of the sets may share a universal addition nucleic acid molecule, and a universal addition nucleic acid molecule may be the first addition nucleic acid molecule to be added to a seed nucleic acid molecule, the last addition nucleic acid molecule to be added in order to form an assembled target sequence or intermediate thereof, and/or any addition nucleic acid molecule in between. In some embodiments, sets P11, . . . , and P1j₁; . . . ; Pk1, . . . , and Pkjₖ; . . . ; and Pi1, . . . , and Pijᵢ do not share any addition nucleic acid molecule. In some embodiments, subsequence sets S11, . . . , and S1j₁; . . . ; Sk1, . . . , and Skjₖ; . . . ; and Si1, . . . , and Sijᵢ do not share any common subsequences. In some embodiments, some or all of subsequence sets S11, . . . , and S1j₁; . . . ; Sk1, . . . , and Skjₖ; . . . ; and Si1, . . . , and Sijᵢ share one or more common subsequences. For example, some or all of the subsequences among the sets may comprise a subsequence that is common among some or all of target sequences S′1, . . . , S′k, . . . , and S′i. A common subsequence may be in the first addition nucleic acid molecule to be added to a seed nucleic acid molecule, in the last addition nucleic acid molecule to be added in order to form an assembled target sequence or intermediate thereof, and/or in any addition nucleic acid molecule in between.

In some embodiments, there is no sequence overlap of 4, 5, 6, 7, 8, 9, 10, 11, 12, 13, 14, 15, 16, 17, 18, 19, 20, or more nucleotides among two or more of target sequences S′1, . . . , S′k, . . . , and S′i. In some embodiments, there is a sequence overlap of 4, 5, 6, 7, 8, 9, 10, 11, 12, 13, 14, 15, 16, 17, 18, 19, 20, or more nucleotides among two or more of target sequences S′1, . . . , S′k, . . . , and S′i. It should be appreciated that sequence overlap among the target sequences does not necessarily mean that some or all of subsequence sets S11, . . . , and S1j₁; . . . ; Sk1, . . . , and Skjₖ; . . . ; and Si1, . . . , and Sijᵢ share one or more common subsequences. On the one hand, the seed and/or addition nucleic acid molecules may be designed such that the overlapping sequence or sequences are distributed in subsequences that also contain non-overlapping sequences, thus making the subsequences different. On the other hand, some or all of subsequence sets S11, . . . , and S1j₁; . . . ; Sk1, . . . , and Skjₖ; . . . ; and Si1, . . . , and Sijᵢ may share a common subsequence. For example, subsequence S11 may have identical sequence as subsequence Skjₖ, but because of the concerted reactions disclosed herein (see e.g., Section IV), the assembly of S11, . . . , and S 1j₁ into S′1 and the assembly of Sk1, . . . , and Skjₖ into S′k may proceed in parallel without interfering with each other, even in cases where molecules containing the two sets of subsequences are partitioned into the same contained reaction volume (e.g., an emulsion droplet). In some aspects, partitioning P11, . . . , and P1j₁ into a reaction volume and Pk1, . . . , and Pkjₖ into a separate reaction volume (see e.g., Section III) would also allow the assembly of S11, . . . , and S 1j₁ into S′1 and the assembly of Sk1, . . . , and Skjₖ into S′k in parallel without interfering with each other.

In some embodiments, an addition nucleic acid molecule disclosed herein can be of any suitable length and/or comprise any suitable composition (e.g., nucleic acid backbone and/or base compositions including modifications), e.g., as long as the addition nucleic acid comprises a 3′ end sequence capable of hybridizing to a 3′ end sequence of a seed nucleotide acid molecule (e.g., as disclosed in Section II-A) or capable of hybridizing to a 3′ end sequence of an assembled product, e.g., a product formed of concerted reactions catalyzed by a ligase, a polymerase, and a Type IIS restriction enzyme (e.g., as disclosed in Section IV).

In some embodiments, an addition nucleic acid molecule is between about 10 and about 20, about 20 and about 30, about 30 and about 40, about 40 and about 50, about 50 and about 60, about 60 and about 70, about 70 and about 80, about 80 and about 90, about 90 and about 100, or more than about 100 nucleotides in length. In some embodiments, an addition nucleic acid molecule is between about 100 and about 200, about 200 and about 300, about 300 and about 400, about 400 and about 500, or more than about 500 in length.

In some embodiments, an addition nucleic acid molecule comprises one or more sequences useful for assembling the target nucleic acid sequence or intermediate thereof and/or the subsequent detection, analysis, and/or use of the assembled sequence, but the one or more useful sequences may be removed (e.g., during the concerted reactions catalyzed by a ligase, a polymerase, and a Type IIS restriction enzyme, e.g., as disclosed in Section IV) and do not need to be present in the assembled target nucleic acid sequence or intermediate thereof. For example, an addition nucleic acid molecule may comprise any one or more of an adapter moiety (e.g., an adapter sequence such as a universal adapter sequence and/or an adapter for sequencing, such as P5 or P7), a tag moiety (e.g., a tag sequence and/or an affinity tag, for hybridization or affinity-based capture onto a support), a primer binding sequence, an amplification sequence, a cleavage site or sequence (e.g., a restriction enzyme recognition sequence and cleavage site), a unique molecular identifier (UMI), a unique identifier (UID), a primer ID, and a barcode, any one or more of which may be unique to the addition polynucleotide or to a subset of addition polynucleotides among a plurality of addition polynucleotides. In some embodiments, an addition polynucleotide comprises a subsequence of a target nucleic acid sequence, e.g., a subsequence in the plus or minus strand of a double-stranded target nucleic acid, where a portion or all of the subsequence in the addition polynucleotide is present in the assembled target nucleic acid sequence or intermediate thereof. In some embodiments, any one or more of an adapter moiety, a tag moiety, a primer binding sequence, an amplification sequence, a cleavage site or sequence, a unique molecular identifier (UMI), a unique identifier (UID), a primer ID, and a barcode may have a sequence that is the same as or distinct from the subsequence, and the sequence may be non-overlapping or partially or completely overlapping with the subsequence.

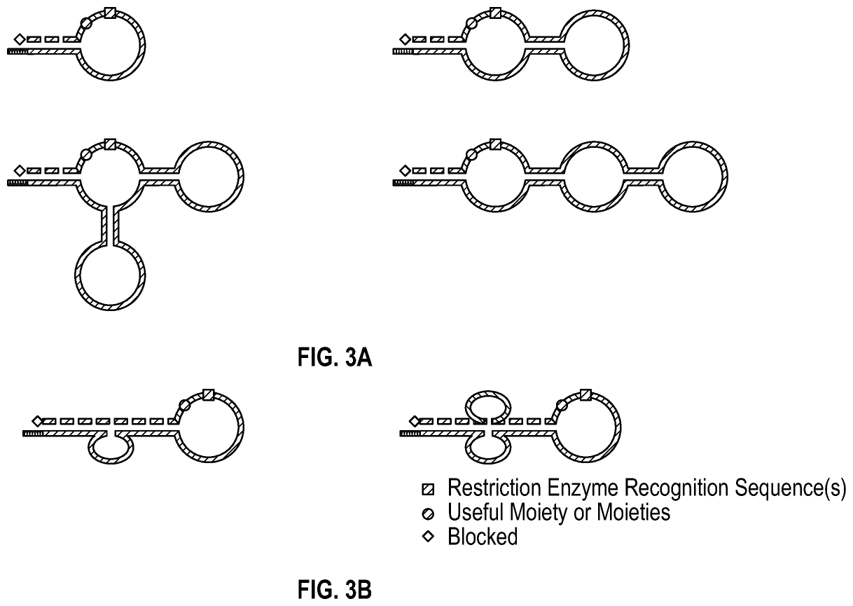

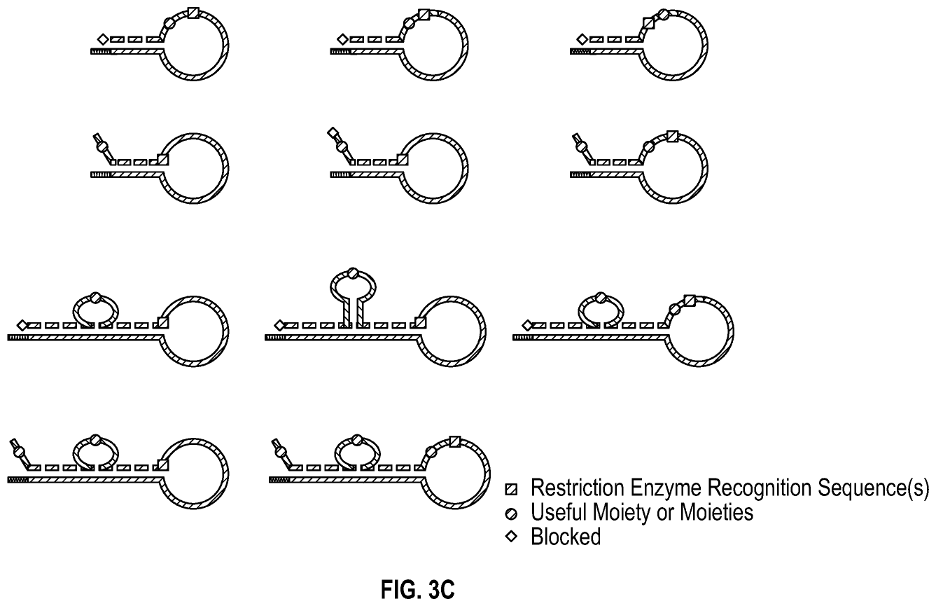

Turning to the figures, FIG. 3A shows exemplary hairpin molecules that can be used as seed and/or addition oligos in assembling a target polynucleotide. The hairpin molecules can include any number of internal hairpins, and in some examples, the one or more paired (“stem”) regions do not provide a restriction enzyme recognition sequence in a double-stranded form that can be cleaved by a restriction enzyme such as a Type IIS enzyme. Thus, in some examples, the hairpin molecules are designed such that cleaving of the hairpin molecules is prevented prior to the subsequence of the hairpin molecule being incorporated into a growing assembled product. In some embodiments, the subsequence of the hairpin molecule includes one or more internal hairpins.

FIG. 3B shows exemplary hairpin molecules that comprise one or more bulges in one or more strands of the stem of a primary hairpin. In some embodiments, the stem of a primary hairpin and/or the stem of an internal hairpin includes one or more bulges in one or more strands of the stem.

FIG. 3C shows exemplary arrangements of the restriction enzyme recognition sequence relative to one or more useful moieties (e.g., sequences), e.g., an adapter, a tag, a primer binding moiety, a cleavage site, a UMI/UID, and/or a barcode. The exemplary hairpin molecules may include a single-stranded 3′ end sequence (black solid line), a subsequence (red solid line) of a target sequence, a Type IIS restriction enzyme recognition sequence (square), and a complementary sequence (red dashed line) capable of hybridizing to all or a portion of the subsequence.

In some embodiments, one or more useful moieties (e.g., sequences) can be between the restriction enzyme recognition sequence and the complementary sequence. In some embodiments, there is no intervening nucleotide (e.g., a “filler” sequence) between the restriction enzyme recognition sequence and the subsequence. In some embodiments, there is a “filler” sequence (gray solid line) between the restriction enzyme recognition sequence and the subsequence. In some embodiments, the restriction enzyme recognition sequence is between the complementary sequence and one or more useful moieties (e.g., sequences).

In some embodiments, the hairpin molecule comprises a 5′ end sequence that does not hybridize to the single-stranded 3′ end sequence or the subsequence. In some embodiments, the 5′ end sequence includes one or more useful moieties (e.g., sequences). In some embodiments, the 5′ end sequence is blocked from ligation, extension (e.g., primer extension), and/or hybridization. In some embodiments, the 5′ end sequence is not blocked from ligation, extension (e.g., primer extension), and/or hybridization, for instance when the 5′ end sequence is not hybridized to the single-stranded 3′ end sequence or the subsequence.

In some embodiments, one or more useful moieties (e.g., sequences) are between the complementary sequence and the restriction enzyme recognition sequence. In some embodiments, one or more useful moieties (e.g., sequences) are included in a 5′ end sequence that does not hybridize to the single-stranded 3′ end sequence or the subsequence. In some embodiments, one or more useful moieties (e.g., sequences) are included in a bulge in the stem region of a hairpin molecule, e.g., on the strand comprising the complementary sequence. In some embodiments, one or more useful moieties (e.g., sequences) are included in an internal hairpin, for instance an internal hairpin in the stem region of a hairpin molecule, e.g., on the strand comprising the complementary sequence.

An addition oligo may comprise any two or more features disclosed herein in a suitable combination. For example, a hairpin addition oligo may comprise a “filler” sequence between the restriction enzyme recognition sequence and the subsequence, one or more internal hairpin structures in the loop region of the primary hairpin structure, one or more bulges and/or hairpin structures in the stem region (on either one or both strands) of the primary hairpin structure, and/or a 5′ end sequence that does not hybridize to the single-stranded 3′ end sequence or the subsequence.

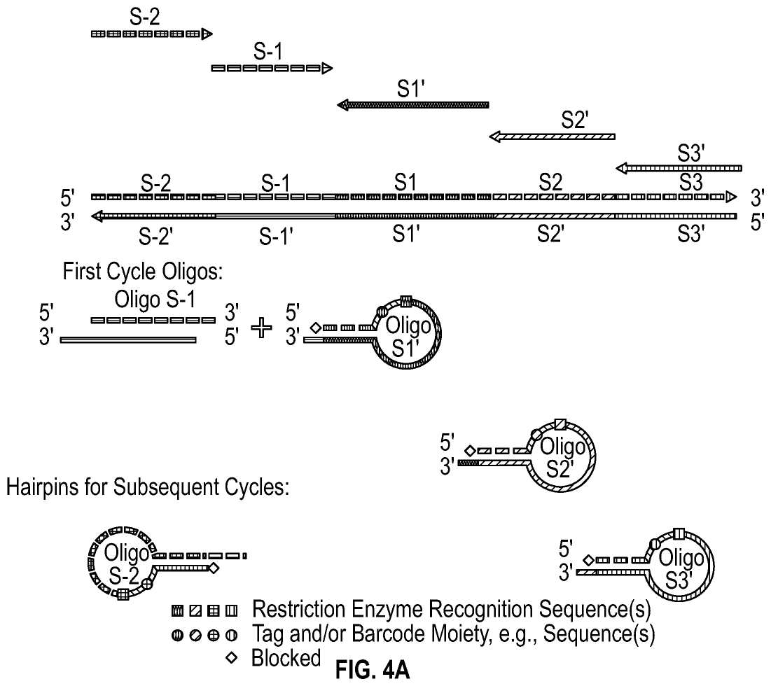

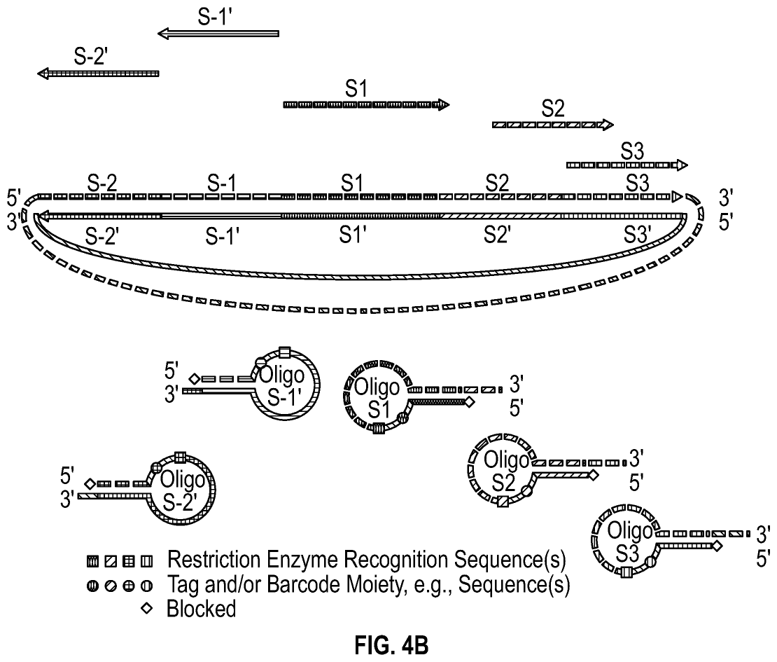

FIG. 4A shows an exemplary target polynucleotide that can be assembled from five subsequences, and exemplary polynucleotides (e.g., oligos) for use during a first cycle of assembling (e.g., using a seed oligo and an addition oligo). The exemplary polynucleotides include a linear Oligo S-1 having a first subsequence S-1, which can be single-stranded or double-stranded. In some examples, Oligo S-1 comprises two single-stranded 3′ end sequences. The exemplary polynucleotides also include Oligo S1′ having in the 3′ to 5′ direction a single-stranded 3′ end sequence, a second subsequence S1′, a Type IIS restriction enzyme recognition sequence (square), a tag and/or barcode sequence (circle), a complementary sequence capable of hybridizing to all or a portion of the second subsequence, and a blocked 5′ end (diamond). The single-stranded 3′ end sequence of Oligo S1′ is complementary to all or a portion of one of the single-stranded 3′ end sequences of Oligo S-1. Oligo Si′ is capable of forming a hairpin molecule with a 3′ overhang, a stem formed by intramolecular nucleotide base pairing between all or a portion of the second subsequence and the complementary sequence, and a loop containing the tag sequence and the Type IIS restriction enzyme recognition sequence. In this configuration, the Type IIS restriction enzyme recognition sequence is single-stranded and therefore the oligo is not cleavable by a Type IIS restriction enzyme.

FIG. 4A also shows exemplary polynucleotides (e.g., hairpin oligos) for use during subsequent cycles of assembly, e.g., adding hairpin oligos to an elongating assembly product. Subsequences in a linear double-stranded target nucleic acid molecule are shown, with arrows indicating the 5′ to 3′ direction. The exemplary polynucleotides include hairpin molecules similar to that used during the first cycle of assembly: Oligo S2′ (having subsequence S2′) and Oligo S3′ (having subsequence S3′) on the right and Oligo S-2 (having subsequence S-2) on the left. The hairpin molecules can also include 3′ overhangs identical or nearly identical to a 5′ end sequence of subsequences in other hairpin molecules. For instance, Oligo S2′ comprises a 3′ overhang complementary or capable of hybridizing to a 3′ end sequence of subsequence S1; Oligo S3′ comprises a 3′ overhang complementary or capable of hybridizing to a 3′ end sequence of subsequence S2; and Oligo S-2 comprises a 3′ overhang complementary or capable of hybridizing to a 3′ end sequence of subsequence S-1′. The sequence complementarity enables incorporation of subsequences through multiple cycles of assembly disclosed herein. The hairpin molecules can each include a unique subsequence, restriction enzyme recognition sequence, and tag barcode sequence. Alternatively, all or some of the hairpin molecules can share one or more subsequences, one or more restriction enzyme recognition sequences, and/or one or more tag sequences.

FIG. 4B shows seed and addition oligos may be designed to assemble subsequences into a circular double-stranded target polynucleotide. Arrows indicate the 5′ to 3′ direction and the figures shows which strand of the circular duplex each subsequence is taken from. In this example, Oligo S3 comprising subsequence S3 is added to an earlier assembled product (e.g., an assembled product comprising sequences of the circular target), Oligo S2 comprising subsequence S2 is added to the product comprising subsequence S3 (and the earlier assembled product), and Oligo S1 comprising subsequence S1 is added to the product comprising subsequence S2 (and S3 and the earlier assembled product). In the other direction of the circle, Oligo S-2′ comprising subsequence S-2′ is added to the earlier assembled product, and Oligo S-1′ comprising subsequence S-1′ is added to the product comprising subsequence S-2′ (and the earlier assembled product). These reactions generate a double-stranded linear product comprising the earlier assembled product and subsequences S-2′, S-1′, S1, S2, and S3, which product comprises a 3′ overhang in the S-1 subsequence and a 3′ overhang in the S1′ subsequence. Because subsequences S-1′ and S1 are complementary at the 5′ ends (which means subsequences S-1 and S1′ are complementary at the 3′ ends), the double-stranded linear product can be circularized to generate the circular double-stranded target polynucleotide.

Certain exemplary individual components of a hairpin oligo are described below.

a. 3′ End Sequence

In some embodiments, the 3′ end sequence of an addition oligo is 1, 2, 3, 4, 5, 6, 7, 8, 9, 10, 11, 12, 13, 14, 15, 16, 17, 18, 19, 20, or more nucleotides in length. In some embodiments, the 3′ end sequence is a single-stranded 3′ overhang.

In some embodiments, the single-stranded 3′ overhang of the first addition oligo to be added to a seed oligo is 1, 2, 3, 4, 5, 6, 7, 8, 9, 10, 11, 12, 13, 14, 15, 16, 17, 18, 19, 20, or more nucleotides in length. In some embodiments, the single-stranded 3′ overhang of subsequent addition oligos, including the last addition oligo to be added to a product assembled in one or more previous cycles of addition in order to form an assembled target sequence or intermediate thereof, is 1, 2, 3, 4, 5, 6, 7, 8, 9, 10, 11, 12, 13, 14, 15, 16, 17, 18, 19, 20, or more nucleotides in length. In some embodiments, the single-stranded 3′ overhang of an addition oligo is 15 or fewer, 12 or fewer, 9 or fewer, 6 or fewer, 3 or fewer, or 2 nucleotides in length, or in any range between the foregoing, such that a polymerase does not extend a 3′ end sequence before a nick on one of the strands is repaired by a ligase.

In particular embodiments, the single-stranded 3′ overhang of subsequent addition oligos, including the last addition oligo, is 2 nucleotides in length, and is complementary to and/or hybridizes to a cleaved product by a Type IIS restriction enzyme. In particular embodiments, the single-stranded 3′ overhang of subsequent addition oligos, including the last addition oligo, is 3 nucleotides in length, and is complementary to and/or hybridizes to a cleaved product by a Type IIS restriction enzyme. In particular embodiments, the single-stranded 3′ overhang of subsequent addition oligos, including the last addition oligo, is 4 nucleotides in length, and is complementary to and/or hybridizes to a cleaved product by a Type IIS restriction enzyme. In particular embodiments, the single-stranded 3′ overhang of subsequent addition oligos, including the last addition oligo, is 5 nucleotides in length, and is complementary to and/or hybridizes to a cleaved product by a Type IIS restriction enzyme. In particular embodiments, the single-stranded 3′ overhang of subsequent addition oligos, including the last addition oligo, is 6 nucleotides in length, and is complementary to and/or hybridizes to a cleaved product by a Type IIS restriction enzyme. In particular embodiments, the single-stranded 3′ overhang of subsequent addition oligos, including the last addition oligo, is 7 nucleotides in length, and is complementary to and/or hybridizes to a cleaved product by a Type IIS restriction enzyme. In particular embodiments, the single-stranded 3′ overhang of subsequent addition oligos, including the last addition oligo, is 8 nucleotides in length, and is complementary to and/or hybridizes to a cleaved product by a Type IIS restriction enzyme. In particular embodiments, the single-stranded 3′ overhang of subsequent addition oligos, including the last addition oligo, is 9 nucleotides in length, and is complementary to and/or hybridizes to a cleaved product by a Type IIS restriction enzyme. In particular embodiments, the single-stranded 3′ overhang of subsequent addition oligos, including the last addition oligo, is 10 nucleotides in length, and is complementary to and/or hybridizes to a cleaved product by a Type IIS restriction enzyme. In particular embodiments, the single-stranded 3′ overhang of subsequent addition oligos, including the last addition oligo, is more than 10 nucleotides in length, and is complementary to and/or hybridizes to a cleaved product by a Type IIS restriction enzyme.

In some embodiments, the 3′ end nucleotide of an addition oligo is capable of being ligated to a 5′ end nucleotide of a seed oligo or a cleaved product by a Type IIS restriction enzyme.

In some embodiments, provided herein is plurality of addition oligos for ordered assembly of a target nucleic acid sequence or intermediate thereof, and each of the plurality of addition oligos comprises a 3′ overhang having a unique sequence among the plurality of addition oligos. For example, a Type IIS restriction enzyme that generates a 2-nt 3′ overhang may be used, and a target nucleic acid sequence may be divided into 17 subsequences S′1 to S′17. A seed oligo P1 comprising S′1 and 16 (i.e., 24) addition oligos P2 to P17 comprising S′2 to S′17, respectively, are constructed. The 3′ overhang of P2 may be of any suitable length that is compatible with a 3′ end sequence of seed oligo P1 to which P2 hybridizes. For example, the 3′ overhang of P2 may be 2, 3, 4, 5, 6, 7, 8, 9, 10, or more than 10 nucleotides in length, and the length is not limited by the distance between the Type II enzyme cleavage site and the enzyme's recognition sequence.

In some examples, the 3′ overhangs of P2 to P17, however, are each 2 nucleotides in length, and each can be one selected from AA, AT, AC, AG, TA, TT, TC, TG, CA, CT, CC, CG, GA, GT, GC, and GG, all in 3′ to 5′ direction. The subsequences and/or the Type IIS restriction enzyme can be selected such that the 2-nt 3′ overhang from a previous reaction cycle specifically hybridizes to one of the 2-nt 3′ overhangs of P2 to P17, in a pre-designed order. In some examples, a template-dependent ligase is used to ligate the nicks formed in the hybridization complexes, and the template-dependency of the ligase ensures that only the correct 3′ overhang (thus the correct addition oligo) is ligated, even when two or more 3′ overhangs with different sequences may hybridize to the same 3′ overhang of a cleaved product from an earlier cycle. Generally, a template-dependent ligase ligates two nucleic acid strands when one strand is aligned adjacently with the other strand onto a template to form a nick, and there is perfect base pairing between the strands and the template, especially at nucleotides close to the nick.

Similarly, a Type IIS restriction enzyme that generates a 3-nt 3′ overhang may be used, and a target nucleic acid sequence may be divided into 82 subsequences, one in each of one seed oligo and 81 (i.e., 34) addition oligos. Likewise, a Type IIS restriction enzyme that generates a 4-nt 3′ overhang may be used, and a target nucleic acid sequence may be divided into 257 subsequences, one in each of one seed oligo and 256 (i.e., 44) addition oligos. A Type IIS restriction enzyme that generates 3′ overhangs that are 5, 6, 7, 8, 9, 10, 11, 12, 13, 14, or 15 nucleotides or even longer may be used.

In some aspects, the concerted action of hybridization due to sequence complementarity and ligase specificity ensures sequence-specific ligation of two ends and/or reduces mismatches. In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

b. Subsequence of a Target Nucleic Acid

An addition nucleic acid may comprise a subsequence as disclosed herein, e.g., in Section I. In some embodiments, when the addition nucleic acid forms a hairpin, the subsequence may form (with a 5′ end sequence of the addition nucleic acid) at least a duplex stem region, and optionally one or more loops. In some embodiments, the entire length of the subsequence is in the duplex stem region, and the loop region comprises a restriction enzyme recognition sequence and optionally one or more tag and/or barcode sequences. In some embodiments, only a portion of the subsequence is in the duplex stem region, and the rest of the subsequence is in the loop region, which further contains a restriction enzyme recognition sequence and optionally one or more tag and/or barcode sequences, as shown in FIG. 3A.

Additional exemplary addition nucleic acid molecules are shown in FIG. 3A, including ones with one or more internal stem-loop structures in the loop region of the primary loop. In some embodiments, the one or more internal stem-loop structures may stabilize the primary loop and the overall structure (e.g., secondary and/or tertiary structures) of the addition oligo, e.g., in cases where the sequence of the primary loop is long, e.g., about 10, about 20, about 30, about 40, about 50, about 60, about 70, about 80, about 90, about 100, about 150, about 200, about 250, about 300, or more than 300 nucleotides in length.

In some embodiments, when the addition nucleic acid forms a hairpin, the duplex stem region may comprise one or more loops or “bulges,” e.g., as shown in FIG. 3B. This in certain aspects may further increase the capacity of the addition oligo, since both the stem region and the loop region may be used to house sequences, thus allowing longer subsequences to be included in the addition oligos. In some embodiments, the 5′ end sequence may also comprise one or more loops or “bulges,” including ones that correspond to one or more loops or “bulges” in the subsequence, e.g., as shown in FIG. 3B. The one or more loops or “bulges” in the 5′ end sequence of the addition oligo may be used to house one or more of an adapter moiety (e.g., an adapter sequence such as a universal adapter sequence and/or an adapter for sequencing, such as P5 or P7), a tag moiety (e.g., a tag sequence and/or an affinity tag, for hybridization or affinity-based capture onto a support), a primer binding sequence, an amplification sequence, a cleavage site or sequence (e.g., a restriction enzyme recognition sequence and cleavage site), a unique molecular identifier (UMI), a unique identifier (UID), a primer ID, and a barcode.

In some embodiments, one or more subsequences disclosed herein are from 10 to about 300 nucleotides, from 20 to about 400 nucleotides, from 30 to about 500 nucleotides, from 40 to about 600 nucleotides, or more than about 600 nucleotides long. In some embodiments, one or more subsequences disclosed herein are between about 10 and about 20, about 20 and about 30, about 30 and about 40, about 40 and about 50, about 50 and about 60, about 60 and about 70, about 70 and about 80, about 80 and about 90, about 90 and about 100, about 100 and about 110, about 110 and about 120, about 120 and about 130, about 130 and about 140, about 140 and about 150, about 150 and about 160, about 160 and about 170, about 170 and about 180, about 180 and about 190, about 190 and about 200, about 200 and about 210, about 210 and about 220, about 220 and about 230, about 230 and about 240, about 240 and about 250, about 250 and about 260, about 260 and about 270, about 270 and about 280, about 280 and about 290, about 290 and about 300, or more than about 300 nucleotides in length.

In some aspects, a subsequence has a 3′ sequence that forms a stem region comprising a duplex with a 5′ sequence of a hairpin oligo, and the 3′ sequence optionally comprises one or more loops and/or bulges, e.g., one or more sequences that do not base pair with a sequence of the 5′ sequence of the hairpin oligo. In some aspects, the 3′ sequence of the subsequence has a length of at least at or about 5 nucleotides, such as at least at or about 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300 or more nucleotides, or within a range defined by any of the foregoing. In some embodiments, the 3′ sequence of the subsequence has a length between at or about 5 nucleotides to at or about 200 nucleotides. In some embodiments, the 3′ sequence of the subsequence is between about 15 and about 100 nucleotides in length.

In some aspects, a subsequence has a sequence that forms a primary loop region of a hairpin oligo. In some aspects, the primary loop region consists of one strand, which optionally comprises one or more internal stem-loop structures. In some aspects, the primary loop region has a length of at least at or about 5 nucleotides, such as at least at or about 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300 or more nucleotides, or within a range defined by any of the foregoing. In some embodiments, the primary loop region has a length between at or about 5 nucleotides to at or about 200 nucleotides. In some embodiments, the primary loop region is between about 15 and about 100 nucleotides in length.

c. Cleavage Enzyme Recognition Sequence and Cleavage Site

In some embodiments, the cleavage enzyme is a restriction enzyme (RE). In some embodiments, a restriction enzyme cleaves DNA or RNA at defined sites upon recognition of a specific nucleotide sequence. There are different classes of REs that are distinct in structure and function. Type I, II, III, and IV REs vary in the sequences they recognize and the sites they cleave in relation to the recognition sequence.

Type IIS REs, a subclass of type II enzymes, generally recognize asymmetrical sequences in double-stranded DNA (dsDNA) and form cleavage sites outside of the recognition sequence, e.g., a Type IIS restriction enzyme can cleave at a defined distance, usually within 1 to 20 nucleotides, outside of its recognition sequence. In some embodiments, these enzymes are monomers that transiently dimerize to cleave both strands of DNA, and many must interact with two copies of the recognition sequence before cleaving dsDNA. Enzyme structure is generally believed to be responsible for the shifted cleavage site. For example, a Type IIS enzyme may comprise a recognition domain at the amino terminus and a cleavage domain in the carboxyl-terminus of the enzyme, and physical separation of the recognition domain from the catalytic, or cleaving, domain produces overhangs that are distinct from the recognition sequence. For example, FokI cleaves 9 and 13 nucleotides away from the recognition sequence on the 5′ to 3′ strand and the complementary strand, respectively.

In some embodiments herein, the activity of Type IIS REs is leveraged to synthesize longer nucleic acid molecules from smaller fragments. For example, fragments of dsDNA with complementary overhangs can be joined by annealing and ligation to form longer strands of DNA with a specific sequence.

Exemplary Type IIS restriction enzymes include but are not limited to AcuI, AlwI, BaeI, BbsI, BbsI-HF, BbvI, BccI, BceAI, BcgI, BciVI, BcoDI, BfiI, BfuAI, BmrI, BpmI, BpuEI, BsaI, BsaI-HF®v2, BsaXI, BseRI, BsgI, BsmAI, BsmBI, BsmBI-v2, BsmFI, BsmI, BspCNI, BspMI, BspQI, BsrDI, BsrI, BtgZI, BtsCI, BtsI-v2, BtsIMutI, CspCI, EarI, EciI, Esp3I, FauI, FokI, HgaI, HphI, HpyAV, MbolI, MlyI, MmeI, MnII, NmeAIII, PleI, SapI, and SfaNI. Recognition sequences and cleavage sites of certain Type IIS are provided in Table 1 below.

TABLE 1

| Type IIS | |
|---|---|
| Restriction | Recognition |
| Enzyme | Sequence and Cleavage Site |
| AcuI | 5′ CTGAAGN₁₆↓3′ |
| | 3′ GACTTCN₁₄↑5′ |
| AlwI | 5′ GGATCN₄↓3′ |
| | 3′ CCTAGN₅↑5′ |
| BaeI | 5′ ↓N₁₀ACNNNNGTAYCN₁₂↓3′ |
| | 3′ ↑N₁₅TGNNNNCATRGN₇↑5′ |
| BbsI | 5′ GAAGACN₂↓3′ |
| | 3′ CTTCTGN₆↑5′ |
| BbsI-HF | 5′ GAAGACN₂↓3′ |
| | 3′ CTTCTGN₆↑5′ |
| BbvI | 5′ GCAGCN₈↓3′ |
| | 3′ CGTCGN₁₂↑5′ |
| BccI | 5′ CCATCN₄↓3′ |
| | 3′ GGTAGN₅↑5′ |
| BceAI | 5′ ACGGCN₁₂↓3′ |
| | 3′ TGCCGN₁₄↑5′ |
| BcgI | 5′ ↓N₁₀CGANNNNNNTGCN₁₂↓3′ |
| | 3′ ↑N₁₂GCTNNNNNNACGN₁₀↑5′ |
| BciVI | 5′ GTATCCN₆↓3′ |
| | 3′ CATAGGN₅↑5′ |
| BcoDI | 5′ GTCTCN₁↓3′ |
| | 3′ CAGAGN₅↑5′ |
| BfiI | 5′ ACTGGGN₅↓3′ |
| | 3′ TGACCCN₄↑5′ |
| BfuAI | 5′ ACCTGCN₄↓3′ |
| | 3′ TGGACGN₈↑5′ |
| BmrI | 5′ ACTGGGN₅↓3′ |
| | 3′ TGACCCN₄↑5′ |
| BpmI | 5′ CTGGAGN₁₆↓3′ |
| | 3′ GACCTCN₁₄↑5′ |
| BpuEI | 5′ CTTGAGN₁₆↓3′ |
| | 3′ GAACTCN₁₄↑5′ |
| BsaI | 5′ GGTCTCN₁↓3′ |
| | 3′ CCAGAGN₅↑5′ |
| BsaI-HF ®v2 | 5′ GGTCTCN₁↓3′ |
| | 3′ CCAGAGN₅↑5′ |
| BsaXI | 5′ ↓N₉ACNNNNNCTCCN₁₀↓3′ |
| | 3′ ↑N₁₂TGNNNNNGAGGN₇↑5′ |
| BseRI | 5′ GAGGAGN₁₀↓3′ |
| | 3′ CTCCTCN₈↑5′ |
| BsgI | 5′ GTGCAGN₁₆↓3′ |
| | 3′ CACGTCN₁₄↑5′ |
| BsmAI | 5′ GTCTCN₁↓3′ |
| | 3′ CAGAGN₅↑5′ |
| BsmBI | 5′ CGTCTCN₁↓3′ |
| | 3′ GCAGAGN₅↑5′ |
| BsmBI-v2 | 5′ CGTCTCN₁↓3′ |
| | 3′ GCAGAGN₅↑5′ |
| BsmFI, | 5′ GGGACN₁₀↓3′ |
| | 3′ CCCTGN₁₄↑5′ |
| BsmI | 5′ GAATGCN₁↓3′ |
| | 3′ CTTACGN₋₁↑5′ |
| BspCNI | 5′ CTCAGN₉↓3′ |
| | 3′ GAGTCN₇↑5′ |
| BspMI | 5′ ACCTGCN₄↓3′ |
| | 3′ TGGACGN₈↑5′ |
| BspQI | 5′ GCTCTTCN₁↓3′ |
| | 3′ CGAGAAGN₄↑5′ |
| BsrDI | 5′ GCAATGN₂↓3′ |
| | 3′ CGTTACN₀↑5′ |
| BsrI | 5′ ACTGGN₁↓3′ |
| | 3′ TGACCN₋₁↑5′ |
| BstF5I | 5′ GGATGN₂↓3′ |
| | 3′ CCTACN₀↑5′ |
| BtgZI | 5′ GCGATGN₁₀↓3′ |
| | 3′ CGCTACN₁₄↑5′ |
| BtsI | 5′ GCAGTGN₂↓3′ |
| | 3′ CGTCACN₀↑5′ |
| BtsCI | 5′ GGATGN₂↓3′ |
| | 3′ CCTACN₀↑5′ |
| BtsI-v2 | 5′ GCAGTGN₂↓3′ |
| | 3′ CGTCACN₀↑5′ |
| BtsIMutI | 5′ CAGTGN₂↓3′ |
| | 3′ GTCACN₀↑5′ |
| CspCI | 5′ ↓N₁₁CAANNNNNGTGGN₁₂↓3′ |
| | 3′ ↑N₁₃GTTNNNNNCACCN₁₀↑5′ |
| EarI | 5′ CTCTTCN₁↓3′ |
| | 3′ GAGAAGN₄↑5′ |
| EciI | 5′ GGCGGAN₁₁↓3′ |
| | 3′ CCGCCTN₉↑5′ |
| Esp3I | 5′ CGTCTCN₁↓3′ |
| | 3′ GCAGAGN₅↑5′ |
| FauI | 5′ CCCGCN₄↓3′ |
| | 3′ GGGCGN₆↑5′ |
| FokI | 5′ GGATGN₉↓3′ |
| | 3′ CCTACN₁₃↑5′ |
| HgaI | 5′ GACGCN₅↓3′ |
| | 3′ CTGCGN₁₀↑5′ |
| HphI | 5′ GGTGAN₈↓3′ |
| | 3′ CCACTN₇↑5′ |
| HpyAV | 5′ CCTTCN₆↓3′ |
| | 3′ GGAAGN₅↑5′ |
| MboII | 5′ GAAGAN₈↓3′ |
| | 3′ CTTCTN₇↑5′ |
| MlyI | 5′ GAGTCN₅↓3′ |
| | 3′ CTCAGN₅↑5′ |
| MmeI | 5′ TCCRACN₂₀↓3′ |
| | 3′ AGGYTGN₁₈↑5′ |
| MnII | 5′ CCTCN₇↓3′ |
| | 3′ GGAGN₆↑5′ |
| NmeAIII | 5′ GCCGAGN₂₁↓3′ |
| | 3′ CGGCTCN₁₉↑5′ |
| PleI | 5′ GAGTCN₄↓3′ |
| | 3′ CTCAGN₅↑5′ |
| SapI | 5′ GCTCTTCN₁↓3′ |
| | 3′ CGAGAAGN₄↑5′ |
| SfaNI | 5′ GCATCN₅↓3′ |
| | 3′ CGTAGN₉↑5′ |

In some embodiments, the Type IIS recognition sequence is not recognized by the enzyme and/or a molecule comprising the Type IIS recognition sequence is not cleaved by the enzyme when the recognition sequence and/or cleavage site are in a substantially single-stranded configuration. In some embodiments, once a single-stranded sequence comprising the Type IIS recognition sequence and/or cleavage site is converted to a duplex, the duplex is recognized by the enzyme and is cleaved. In some embodiments, the Type IIS enzyme is one that generates a 3′ overhang after cleavage, e.g., AcuI, BaeI, BcgI, BciVI, BfiI, BmrI, BpmI, BpuEI, BsaXI, BseRI, BsgI, BsmI, BspCNI, BsrDI, BsrI, BstF5I, BtsI, BtsCI, BtsI-v2, BtsIMutI, CspCI, EciI, HphI, HpyAV, MbolI, MmeI, MnII, or NmeAIII.

In some embodiments, a cleavage enzyme (e.g., restriction enzyme) recognition sequence in an addition oligo directly abuts the subsequence of a target nucleic acid sequence, e.g., as shown in FIG. 3C, first row, first hairpin. In some examples, one or more or all of a plurality of addition oligos may comprise a recognition sequence of an enzyme that cuts at position 0 (N₀) in the 3′ to 5′ direction. For instance, one or more or all of a plurality of addition oligos may comprise a recognition sequence of one or more of BsrDI, BstF5I, BtsI, BtsCI, BtsI-v2, and BtsIMutI. Because these Type IIS restriction enzymes cut at N₀ in the 3′ to 5′ direction and generate a double-stranded end having a 3′ overhang, the recognition sequence is removed after enzyme cleavage, leaving no “scar” in the subsequence. In some embodiments, there is no intervening nucleotide between the subsequence and the recognition sequence.

In some embodiments, there is one or more intervening nucleotides between a cleavage enzyme (e.g., restriction enzyme) recognition sequence and a subsequence of a target nucleic acid sequence in an addition oligo, e.g., as shown in FIG. 3C, first row, third and second hairpins. In some examples, one or more or all of a plurality of addition oligos may comprise a recognition sequence of an enzyme that cuts at a position further away from the recognition sequence than N₀ in the 3′ to 5′ direction and generates a double-stranded end having a 3′ overhang. Type IIS restriction enzymes that cut into a subsequence would leave a scar in a subsequence, and sequences in these scars may be lost during assembly. In some embodiments, a sequence in a scar of an n^{th} cycle subsequence may be provided in an (n+1)^{th} cycle subsequence. In some embodiments, an n^{th} cycle addition oligo may be designed such that it comprises a “filler” sequence of one or more nucleotides such that the enzyme cuts out the filler sequence and leaves no scar in an assembled sequence comprising the n^{th} cycle subsequence. In some embodiments, a filler sequence may comprise one or more useful sequences, e.g., as disclosed in Section II-B-d.

In some embodiments, a Type IIS restriction enzyme may cut within the recognition sequence (e.g., BsmI and BsrI) and leave one or more nucleotides of the recognition sequence in a cleaved product comprising an n^{th} cycle subsequence and upon addition of an (n+1)^{th} cycle subsequence, in the assembled sequence. In some examples, the addition oligos may be designed such that the one or more nucleotides of the recognition sequence are identical to those in the (n+1)^{th} cycle subsequence at the junction between the n^{th} and (n+1)^{th} cycle subsequences.

In some embodiments, provided herein is a plurality of addition nucleic acid molecules, each of which comprising a recognition sequence of the same Type IIS restriction enzyme. In some embodiments, provided herein is a plurality of addition nucleic acid molecules, at least two of which comprising recognition sequences of different Type IIS restriction enzymes.

d. Additional Useful Moieties

In some embodiments, one or more of a seed nucleic acid and/or an addition nucleic acid disclosed herein may comprise one or more moieties (e.g., sequences) useful for assembling a target nucleic acid sequence or intermediate thereof and/or useful for the subsequent detection, analysis, and/or use of the assembled sequence. The one or more moieties (e.g., sequences) may be in any suitable region of a seed nucleic acid and/or an addition nucleic acid, for example, as shown in FIG. 2 and FIGS. 3A-3C.

In some embodiments, the one or more moieties (e.g., sequences) may be removed and do not need to be present in the assembled target nucleic acid sequence or intermediate thereof. In some embodiments, the one or more moieties (e.g., sequences) may remain in the assembled target nucleic acid sequence or intermediate thereof and do not need to be removed and/or are preferably not removed.

For example, one or more of a seed nucleic acid and/or an addition nucleic acid disclosed herein may comprise any one or more of an adapter moiety (e.g., an adapter sequence such as a universal adapter sequence and/or an adapter for sequencing, such as P5 or P7), a tag moiety (e.g., a tag sequence and/or an affinity tag, for hybridization or affinity-based capture onto a support), a primer binding sequence, an amplification sequence, a cleavage site or sequence (e.g., a restriction enzyme recognition sequence and cleavage site), a unique molecular identifier (UMI), a unique identifier (UID), a primer ID, and a barcode. In some examples, any one or more of the useful moieties (e.g., sequences) may be unique to the seed nucleic acid and/or addition nucleic acid, or may be unique to a subset of a plurality of seed nucleic acid(s) and/or addition nucleic acid(s). In some examples, any one or more of the useful moieties (e.g., sequences) may be common to two or more or all of a plurality of seed nucleic acid(s) and/or addition nucleic acid(s).

In some embodiments, any one or more of the useful moieties (e.g., sequences) may have a sequence that is the same as or distinct from a subsequence in a seed nucleic acid and/or an addition nucleic acid. In some embodiments, any one or more of the useful sequences may be non-overlapping or partially or completely overlapping with a subsequence in a seed nucleic acid and/or an addition nucleic acid.

In some embodiments, the one or more of the useful sequences comprise a barcode sequence. In some aspects, the barcode provides information for identification of a nucleic acid molecule or a set of nucleic acid molecules. In some aspects, the barcode comprises a label, or identifier, that conveys or is capable of conveying information, such as a nucleic acid sequence that is used to identify, e.g., a single bead or a population of beads, a single nucleic acid sequence or a set of nucleic acid sequences, and/or a single nucleic acid molecule or a set of nucleic acid molecules. Barcodes can be linked to a nucleic acid molecule and/or a bead and/or another moiety or structure using ligation, amplification, and/or other chemical or biological conjugation methods. A particular barcode can be unique relative to other barcodes. A barcode can be attached to a nucleic acid molecule and/or a bead and/or another moiety or structure in a reversible or irreversible manner. Barcodes can allow for identification and/or quantification of individual sequencing-reads (e.g., a barcode can be or can include a unique molecular identifier or UMI).

Although the barcode sequences described herein can be any suitable length, barcoded sequences are typically between about 5 and about 30 nucleotides in length, e.g., between about 10 and about 25 nucleotides in length, and can serve as is a unique identifier (e.g., of a single nucleic acid sequence or a set of nucleic acid sequences, and/or a single nucleic acid molecule or a set of nucleic acid molecules), is an error-checking barcode, and/or can be used as a tag (e.g., a capture tag sequence), as non-limiting examples.

In some aspects, one or more of the polynucleotides disclosed herein, e.g., a seed oligo, an addition oligo, a terminal oligo, and/or a capture oligo, include one or more barcode(s), e.g., at least two, three, four, five, six, seven, eight, nine, 10, or more barcodes. Barcodes can spatially-resolve molecular components found in a sample or mixture. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

e. Complementary Sequence

In some embodiments, one or more of a seed nucleic acid and/or an addition nucleic acid disclosed herein may comprise one or more sequences that are complementary or able to hybridize to one or more other sequences in a seed nucleic acid or an addition nucleic acid. In some embodiments, the sequences are completely complementary. In some embodiments, the sequences are substantially complementary. In some embodiments, the terms “complementary” or “substantially complementary” encompasses the hybridization or base pairing or the formation of a duplex between nucleotides or nucleic acids, such as, for instance, between the two strands of a double stranded DNA molecule, or between two or more segments of a single-stranded nucleic acid, e.g., one that is capable of forming a stem-loop structure upon hybridization of the two or more segments. Complementary nucleotides are, generally, A and T (or A and U), or C and G. Two single-stranded nucleic acid molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when a nucleic acid strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary.

The term “duplex” encompasses the pairing involving one or more nucleoside analogs (e.g., pairing between two analogs, or pairing between a nucleoside and an analog), such as deoxyinosine, nucleosides with 2-aminopurine bases, PNAs, and the like, that may be employed. In some embodiments, the complementary sequence of an addition oligo disclosed herein comprises one or more nucleoside analogs.

In some embodiments, one or more of a seed nucleic acid and/or an addition nucleic acid disclosed herein may comprise sequences that form a duplex, e.g., at least two sequences that are fully or partially complementary undergo Watson-Crick type base pairing among all or most of their nucleotides so that a stable complex is formed. In some embodiments, one or more of a seed nucleic acid and/or an addition nucleic acid disclosed herein may comprise a stem region that comprises a stable duplex formed by annealing or hybridization of two or more sequences of the same molecule, e.g., a single-stranded oligo.

In some embodiments, one or more of a seed nucleic acid and/or an addition nucleic acid disclosed herein may comprise a duplex structure that is not destroyed by a stringent wash, e.g., conditions including temperature of about 5° C. less that the Tₘ of a strand of the duplex and low monovalent salt concentration, e.g., less than 0.2 M, or less than 0.1 M. In some embodiments, a stem region of a seed nucleic acid and/or an addition nucleic acid is not destroyed by a stringent wash.

In some embodiments, one or more of a seed nucleic acid and/or an addition nucleic acid disclosed herein may comprise a duplex structure that is perfectly matched, e.g., the sequences making up the duplex form a double stranded structure with one another such that every nucleotide in each strand undergoes Watson-Crick base pairing with a nucleotide in the other strand.

In some embodiments, one or more of a seed nucleic acid and/or an addition nucleic acid disclosed herein may comprise a mismatch in a duplex in which one or more nucleotides in one sequence do not undergo Watson-Crick bonding with one or more nucleotides in the other sequence. In some embodiments, the complementary sequence of an addition oligo disclosed herein comprises one or more mismatches with a sequence in the 3′ of a subsequence of a target nucleic acid. In some embodiments, the complementary sequence of an addition oligo comprises one or more one or more loops (e.g., in a stem-loop structure) or bulges, e.g., as shown in FIG. 3C, third and fourth rows. The one or more loops or bulges may be used to house one or more useful moieties, such as an adapter moiety (e.g., an adapter sequence such as a universal adapter sequence and/or an adapter for sequencing, such as P5 or P7), a tag moiety (e.g., a tag sequence and/or an affinity tag, for hybridization or affinity-based capture onto a support), a primer binding sequence, an amplification sequence, a cleavage site or sequence (e.g., a restriction enzyme recognition sequence and cleavage site), a unique molecular identifier (UMI), a unique identifier (UID), a primer ID, and a barcode.

In some aspects, the complementary sequence, optionally comprising one or more loops and/or bulges, has a length of at least at or about 5 nucleotides, such as at least at or about 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300 or more nucleotides, or within a range defined by any of the foregoing. In some embodiments, the complementary sequence, optionally comprising one or more loops and/or bulges, has a length between at or about 5 nucleotides to at or about 200 nucleotides. In some embodiments, the complementary sequence, optionally comprising one or more loops and/or bulges, is between about 15 and about 100 nucleotides in length.

In some aspects, the stem region of a hairpin oligo disclosed herein comprises at least at or about 5, such as at least at or about 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300 or more base pairs (e.g., nucleosides that form a base pair, excluding bases in one or more loops and/or bulges), or within a range defined by any of the foregoing. In some embodiments, the stem region comprises at or about 5 nucleotides to at or about 200 base pairs. In some embodiments, the stem region comprises between about 15 and about 100 base pairs.

f. 5′ End Sequence

In some embodiments, the hairpin molecule does not comprise a 5′ end sequence that does not hybridize to the single-stranded 3′ end sequence or the subsequence. In some embodiments, the 5′ end sequence of an oligo (e.g., an addition oligo that is not a terminal oligo) is blocked from ligation, e.g., the 5′ nucleotide is dephosphorylated. In some embodiments, the 5′ end sequence of an oligo (e.g., a terminal oligo) permits ligation, e.g., the 5′ nucleotide is phosphorylated.

In some embodiments, the hairpin molecule comprises a 5′ end sequence that does not hybridize to the single-stranded 3′ end sequence or the subsequence. In some embodiments, the 5′ end sequence includes one or more useful moieties (e.g., sequences). In some embodiments, the 5′ end sequence is blocked from ligation (e.g., the 5′ nucleotide is dephosphorylated), extension (e.g., primer extension), and/or hybridization. In some embodiments, the 5′ end sequence is not blocked from ligation, extension (e.g., primer extension), and/or hybridization, for instance when the 5′ end sequence is not hybridized to the single-stranded 3′ end sequence or the subsequence.

In some aspects, the 5′ end sequence that does not hybridize to the single-stranded 3′ end sequence or the subsequence has a length of at least at or about 1, 2, 3, 4, or 5 nucleotides, such as at least at or about 10, 15, 20, 25, 30, 35, 40, 45, 50, 60, 70, 80, 90, 100 or more nucleotides, or within a range defined by any of the foregoing. In some embodiments, the 5′ end sequence has a length between at or about 5 nucleotides to at or about 200 nucleotides. In some embodiments, the 5′ end sequence is between about 10 and about 50 nucleotides in length.

In some embodiments, one or more useful moieties (e.g., sequences) are included in a 5′ end sequence that does not hybridize to the single-stranded 3′ end sequence or the subsequence, e.g., as shown in FIG. 3C.

### III. Partitioning of Nucleic Acid Molecules

In certain exemplary embodiments, oligonucleotide sequences are provided on a support (e.g., bead or solid substrate), such as an array or a bead. Oligonucleotide sequences may be synthesized on a support (e.g., bead or solid substrate) in an array format, e.g., a microarray of single stranded DNA segments synthesized in situ on a common substrate wherein each oligonucleotide is synthesized on a separate feature or location on the substrate. Arrays may be constructed, custom ordered, or purchased from a commercial vendor. Various methods for constructing arrays are well known in the art. For example, methods and techniques applicable to synthesis of construction and/or selection oligonucleotide synthesis on a solid support, e.g., in an array format have been described, for example, in WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752 and Zhou et al., Nucleic Acids Res. 32: 5409-5417 (2004). In an exemplary embodiment, construction and/or selection oligonucleotides may be synthesized on a solid support using maskless array synthesizer (MAS). Other methods synthesizing construction and/or selection oligonucleotides include, for example, light-directed methods utilizing masks, flow channel methods, spotting methods, pin-based methods, and methods utilizing multiple supports.

Barcoded bead libraries can be constructed from chips by emulsion PCR. Emulsion methods are known to those of skill in the art. Methods and reagents useful in the present disclosure are described in Shendure et al., Science 309(5741):1728-32, Williams et al., Nature Methods 3:545-550 (2006), Diehl et al., Nature Methods 3:551-559 (2006) and Schutze et al., Analytical Biochemistry 410:155-157 (2011) each of which are hereby incorporated by reference in their entireties. Designed barcodes can be synthesized on chips with common PCR primers and a nuclease recognition site on the 3′ end internal to the PCR primer. The library is clonally amplified on beads using standard limited dilution emulsion PCR techniques such that only one barcode is amplified onto beads leaving a plurality of beads with no amplification product. The beads are then de-emulsified, and processed by a nuclease to remove the common PCR primer located distal to the attachment point. De-emulsification protocols are known to those of skill in the art. See, for example, Schutze et al., Analytical Biochemistry 410:155-157 (2011). The DNA on the beads is then made single stranded by standard techniques such as NaOH elution. The beads may be further enriched using standard bead enrichment techniques used for high-throughput sequencing. These orthogonal bead libraries can be used for many assembly reactions depending on the scale of synthesis of the oligonucleotides or emulsion PCR. Other suitable methods for bead library construction may be used, for example, as disclosed in U.S. Pat. Nos. 9,822,401, 10,533,218, and 10,544,456, All of which are incorporated by reference in their entireties.

In certain exemplary aspects, oligonucleotide sequences are provided which include a capture tag or barcode sequence. The capture tag or barcode is used to identify or encode a group or collection of oligonucleotide sequences. The capture tag or barcode sequence may be randomly generated or it may be a predesigned sequence. According to one aspect, a plurality of oligonucleotide sequences may have the same capture tag or barcode sequence, and accordingly, form an oligonucleotide set. The set of oligonucleotides which may be within a larger collection of oligonucleotides may be localized or co-located by using the capture tag or barcode.

In some embodiments, a plurality of polynucleotides (e.g., oligos) comprising subsequences of one or more target nucleic acid sequences, for example, a plurality of polynucleotides (e.g., oligos) in a mixture, are partitioned into one or more partitions. In some embodiments, the plurality of polynucleotides (e.g., oligos) are localized, e.g., by direct or indirect attachment such as via covalent-bonding and/or via hybridization, onto one or more supports, e.g., a bead or a solid substrate. In some embodiments, one or more subsets of the plurality of polynucleotides (e.g., oligos) are captured, sequestered or otherwise contained within one or more reaction volumes, such as a droplet, e.g., an emulsion droplet. In some embodiments, the subset of the plurality of polynucleotides (e.g., oligos) in a reaction volume is assembled into one or more assembled nucleic acid molecules comprising one or more target nucleic acid sequences.

In some embodiments, the partitions can be flowable within fluid streams. In some embodiments, the partitions comprise micro-vesicles that have an outer barrier surrounding an inner fluid center or core. In some embodiments, the partitions may comprise a porous matrix that is capable of entraining and/or retaining materials within its matrix. In some embodiments, the partitions can be droplets of a first phase within a second phase, wherein the first and second phases are immiscible. In some embodiments, the partitions can be droplets of aqueous fluid within a non-aqueous continuous phase (e.g., oil phase). In some embodiments, the partitions can be droplets of a non-aqueous fluid within an aqueous phase. In some embodiments, the partitions may be provided in a water-in-oil emulsion or oil-in-water emulsion. In some embodiments, the partitions can comprise gel beads. A variety of different vessels are described in, for example, U.S. Pat. No. 9,689,024, which is entirely incorporated herein by reference for all purposes. Emulsion systems for creating stable droplets in non-aqueous or oil continuous phases are described in, for example, U.S. Pat. No. 9,012,390, which is entirely incorporated herein by reference for all purposes. Gel beads and uses thereof are described in, for example, U.S. Pat. No. 10,876,147, which is entirely incorporated herein by reference for all purposes.

In some embodiments, disclosed herein is a method comprising capturing, localizing, and/or sequestering one or more subsets of a plurality of polynucleotides (e.g., oligos) onto or into one or more structures and/or partitions, thereby isolating or separating the one or more subsets, e.g., from one or more other subsets of the plurality of polynucleotides. In some embodiments, the one or more subsets are enriched on or in the one or more structures and/or partitions, e.g., a bead or a solid substrate. In some embodiments, the one or more subsets are captured, localized, and/or sequestered via hybridization to one or more predesigned sequences, e.g., one or more capture probes or barcodes on a bead or a planar substrate, that are unique to the one or more subsets. In some embodiments, each subset of polynucleotides (e.g., oligos) is captured, localized, and/or sequestered via hybridization to a predesigned sequence, e.g., a capture probe or barcode on a bead or a planar substrate, that is unique to the subset. For example, each subset may be uniquely identified among all subsets of the plurality of polynucleotides or distinguished from any other subset of the plurality of polynucleotides by the predesigned sequence that corresponds to the subset.

For example, polynucleotides comprising subset A1, . . . , Ai, subset B1, . . . , Bj, and/or subset C1, . . . , Ck may be contacted with one or more predesigned sequences, e.g., one or more capture probes or barcodes on a bead or a planar substrate, wherein i, j, and k are positive integers independent of one another. In some examples, all of polynucleotides A1, . . . , Ai, B 1, . . . , Bj, C1, . . . , and Ck comprise one or more sequences that hybridize to a capture probe Px on bead X, therefore all three subsets can be captured on bead X. The one or more sequences in polynucleotides A1, . . . , Ai, B1, . . . , Bj, C1, . . . , and Ck can be the same or different. For example, all or a subset of the polynucleotides can comprise a universal capture tag or barcode sequence that hybridizes to capture probe Px. In another example, the polynucleotides may comprise two or more different capture tag or barcode sequences that hybridize to capture probe Px, e.g., the two or more different capture tag or barcode sequences may hybridize to different regions of Px. In yet another example, the polynucleotides may comprise two or more different capture tag or barcode sequences that hybridize to capture probe Px and/or one or more capture probes Px′ of a different sequence on bead X.

In some examples, the polynucleotides are contacted with beads X and Y comprising capture probes Px and Py, respectively. One or more of subsets A, B, and C can hybridize to capture probe Px and/or Py. For example, subset A can hybridize to capture probe Px while subsets B and C hybridize to capture probe Py. In another example, subsets A and B can hybridize to capture probe Px (e.g., subset A and subset B hybridize to different regions of Px), while subsets B and C can hybridize to capture probe Py (e.g., subset B and subset C hybridize to different regions of Py). In other words, a sequence in Px and a sequence in Py may hybridize to the same one or more polynucleotides, e.g., Px and Py may share a common sequence. In some examples, the polynucleotides are contacted with beads X, Y, and Z comprising capture probes Px, Py, and Pz, respectively. In some examples, subset A hybridizes to capture probe Px, subset B hybridizes to capture probe Py, and subset C hybridizes to capture probe Pz. Again, one or more of subsets A, B, and C can hybridize to capture probe Px and/or Py and/or Pz, and any two or more of Px, Py, and Pz may share a common sequence that hybridizes to polynucleotides of one or more of subsets A, B, and C.

In some embodiments, two or more polynucleotides in subset A1, . . . , Ai, subset B1, . . . , Bj, and/or subset C1, . . . , Ck may comprise one or more universal sequences. In some embodiments, subset A1, . . . , Ai, subset B1, . . . , Bj, and/or subset C1, . . . , Ck may comprise one or more universal polynucleotides.

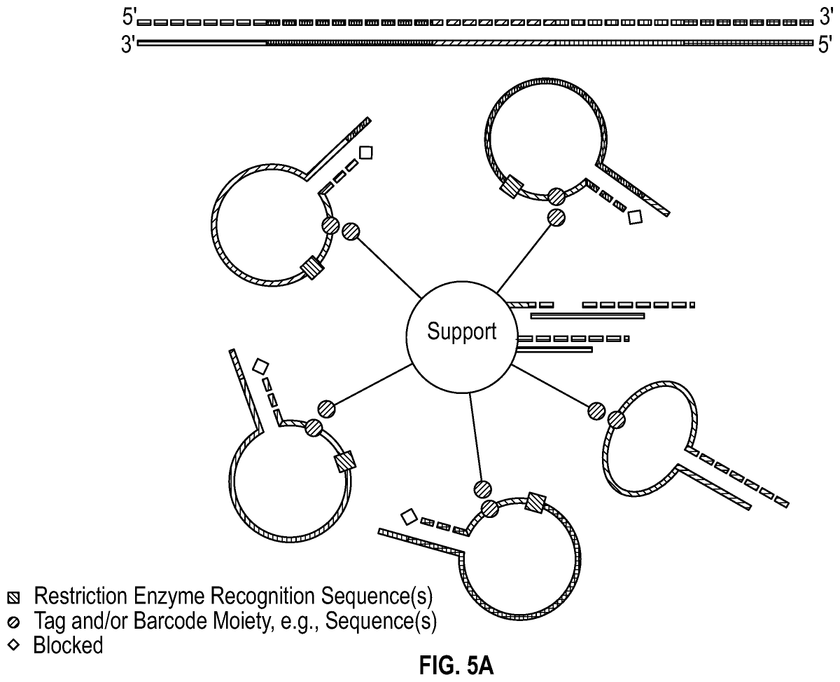

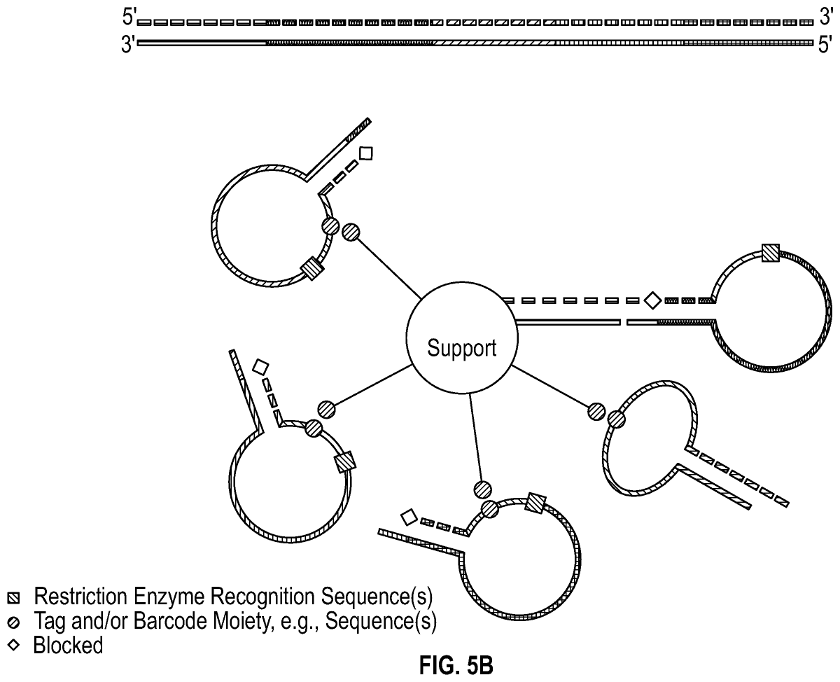

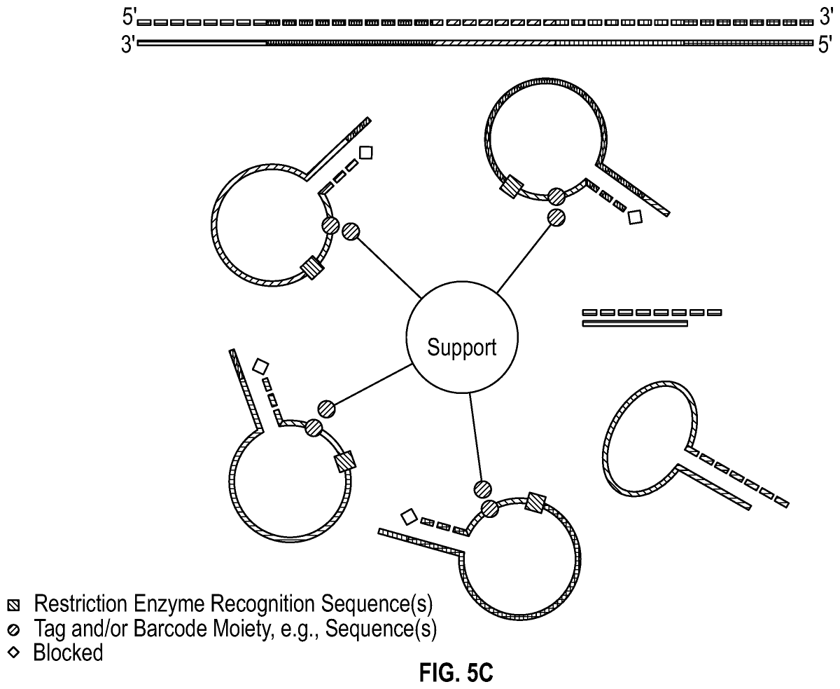

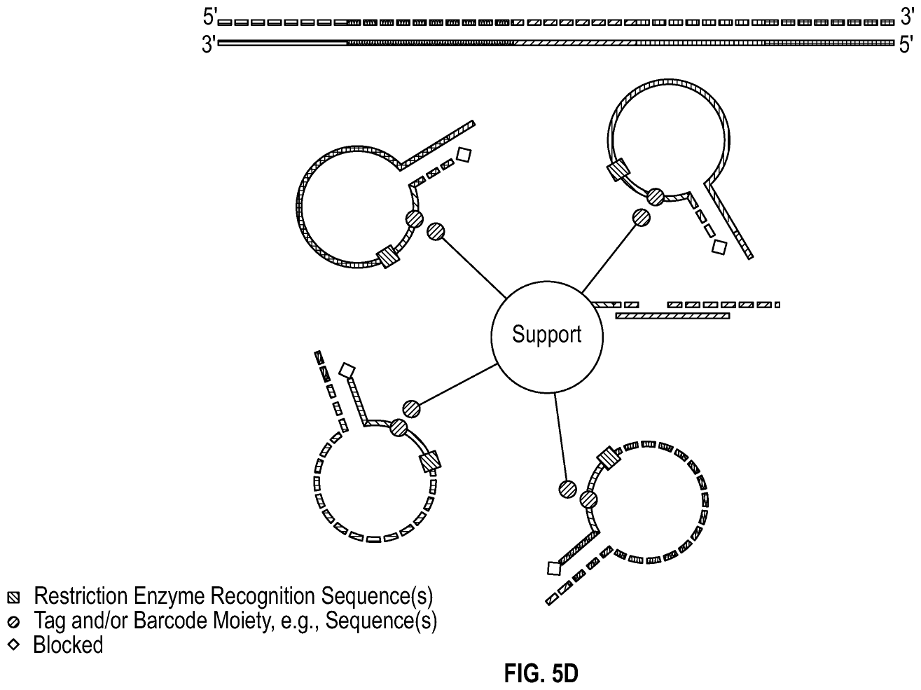

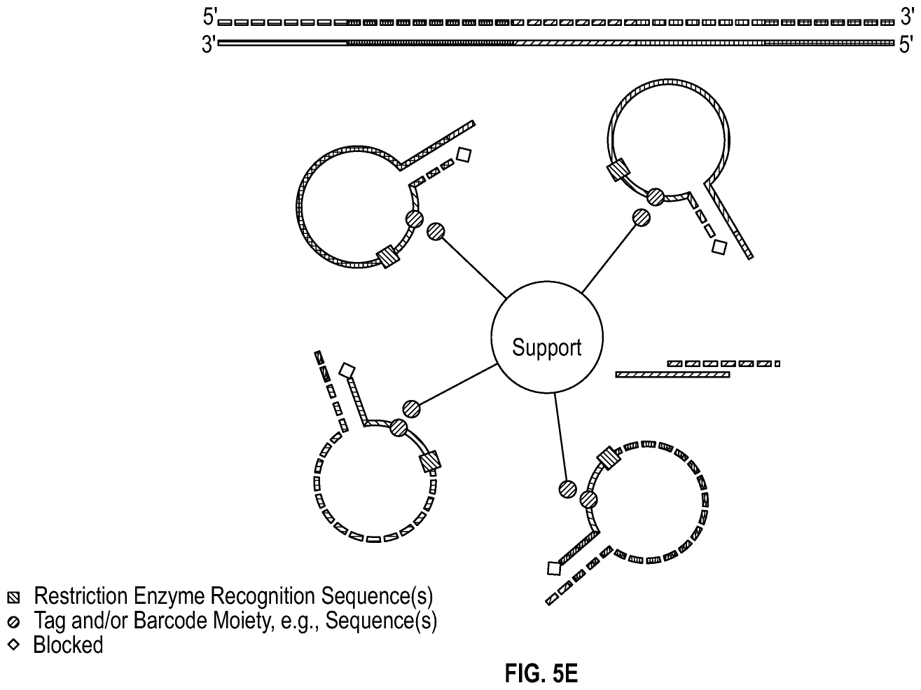

Turning to the figures, FIG. 5A shows an exemplary target polynucleotide to be assembled (top) and a support (e.g., bead or solid substrate) that can be used to capture hairpin molecules by their tag sequences, for assembling subsequences in the hairpin molecules to form one or more target sequences. For example, the target polynucleotide can be assembled in a unidirectional fashion. The first subsequence can be included in a linear polynucleotide. In some embodiments, the linear polynucleotide has a single-stranded 3′ end sequence that hybridizes to a sequence attached to the support. In other embodiments, the linear polynucleotide is covalently attached to the support either directly or indirectly. In other embodiments, the first subsequence is included in a hairpin molecule that comprises a tag moiety such as a capture tag sequence, e.g., the tag sequence can be captured via hybridization to a capture probe sequence attached to the support.

In FIG. 5A, hairpin molecules containing other subsequences to be incorporated into a target sequence are shown. In some examples, the hairpin molecules are captured by the support (e.g., bead or solid substrate) via hybridization between the tag sequences of the hairpin molecules and capture probe sequences attached to the support. In some examples, all hairpin molecules include the same tag sequence, and the support does not include capture probe sequences for other tag sequences. In certain aspects, the use of the same tag sequence across hairpin molecules allows for the capture of hairpin molecules whose subsequences are intended to be incorporated into the same target sequence. In certain aspects, the use of a support (e.g., bead or solid substrate) that specifically captures a tag sequence allows for the hairpin molecules to be isolated from hairpin molecules not to be incorporated into the same target sequence.

FIG. 5B shows an exemplary method of using a support (e.g., bead or solid substrate) to capture polynucleotides with subsequences to be incorporated into a target sequence. In some examples, the first subsequence is included in a hairpin molecule that includes a tag sequence, and the tag sequence is captured via hybridization to a capture probe sequence attached to the support. In some examples, the first subsequence may be directly attached to the support, and the single-stranded 3′ end sequence captures via hybridization the hairpin molecule containing the second subsequence. In this configuration, the hairpin molecule containing the second subsequence need not have a tag sequence. Other hairpin molecules are shown captured by the support via hybridization between the tag sequences of the hairpin molecules and capture probe sequences attached to the support. In some examples, all hairpin molecules include the same tag sequence, and the support does not include capture probe sequences for other tag sequences. The hairpin seed oligo and the hairpin addition oligos may be released from the bead, e.g., by heating.

FIG. 5C shows an exemplary method of using a support (e.g., bead or solid substrate) to capture polynucleotides with subsequences to be incorporated into a target sequence. In some examples, the first subsequence can be included in a hairpin molecule or a linear polynucleotide, which is not attached to the support. Other hairpin molecules are shown captured by the support via hybridization between the tag sequences of the hairpin molecules and capture probe sequences attached to the support. To assemble the target nucleotide, seed oligo molecules (e.g., oligos comprising the first subsequence) can be provided after capture of the hairpin molecules, either before, during, or after the beads (with oligos captured thereon) are partitioned into emulsion droplets. For example, seed oligo molecules, including common or universal seed oligos, may be provided in a bulk aqueous solution which is partitioned into a plurality of aqueous droplets containing at most one bead per droplet.

FIG. 5D shows an exemplary method of using a support (e.g., bead or solid substrate) to capture polynucleotides for bidirectional assembly of a target sequence. In some examples, a linear polynucleotide is captured via hybridization to a sequence attached to the support. The target sequence is assembled by extending from both sides of the linear polynucleotide. Hairpin molecules are shown captured by the support via hybridization between the tag sequences of the hairpin molecules and capture probe sequences attached to the support.

FIG. 5E shows an exemplary method of using a support (e.g., bead or solid substrate) to capture polynucleotides for bidirectional assembly of a target polynucleotide. In some examples, a linear seed oligo is not captured by the support, and can be provided after capture of the hairpin molecules, either before, during, or after the beads (with oligos captured thereon) are partitioned into emulsion droplets. For example, seed oligo molecules, including common or universal seed oligos, may be provided in a bulk aqueous solution which is partitioned into a plurality of aqueous droplets containing at most one bead per droplet.

In some embodiments, the oligonucleotide set corresponds to a particular target nucleic acid sequence. In some embodiments, the plurality of oligonucleotide subsequences defining an oligonucleotide set is isolated within an emulsion droplet.

In some embodiments, a barcoded library is generated, such as a bead-based library having barcoded oligonucleotides attached thereto using methods known to those of ordinary skill. For example, individual biotinylated oligonucleotides can be synthesized, attached to beads having streptavidin attached thereto (streptavidin beads), and subsequently mixed to form a library of barcoded beads. Barcode sequences can be arbitrary sequences, or they can be designed to be orthogonal to one another. Attachment chemistries to the beads can vary using chemistries known to those of skill in the art such as biotin, carboxylation, and the like). Barcoded bead libraries as described herein can be repeatedly used for assembly methods described herein.

In some embodiments, the barcode sequences are the same as or are common to the bead. Accordingly, a bead is provided having a plurality of barcode sequences having a common nucleic acid sequence. The barcode sequences are able to bind a plurality of oligonucleotides sharing the complement to the common nucleic acid barcode sequence. In this exemplary manner, only oligonucleotides having the same complementary barcode sequence can bind to same barcode sequences on the bead. If a particular set of assembly oligonucleotides (e.g., seed and/or addition oligos) are provided with the same barcode sequence, the set of assembly oligonucleotides will bind to the same bead. Therefore, the set of assembly oligonucleotides can be located within an emulsion droplet for the making of a target nucleic acid.

In a certain aspect, a library of beads with captured oligos is emulsified in a buffer and enzyme mixture that contains one or more enzymes, one or more oligos in solution (e.g., a common or universal seed oligo and/or terminal oligo, and/or one or more probes and/or primers), and/or additional reagents known to those of skill in the art and as described herein, to facilitate assembly. In some embodiments, the enzyme mixture comprises one or more ligases, one or more polymerases, one or more restriction enzymes such as Type IIS enzymes, one or more other nucleases such as exonucleases, and/or one or more other enzymes.

In some embodiments, the emulsified mixture contains a plurality of beads which may be from at least 100 beads, at least 1000 beads, at least 10,000 beads, at least 100,000 beads, at least 1,000,000 beads and higher. In some embodiments, each bead of the plurality is unique, for example, each bead comprises a unique barcode and/or a capture oligo sequence. In some embodiments, the beads can be redundant, e.g., two or more beads of the plurality may comprise the same barcode and/or the same capture oligo. In some embodiments, the plurality of beads comprise two or more copies of each bead, wherein each bead comprises a separate assembly reaction compartment. In some embodiments, a bead of the plurality comprises two or more copies of a barcode and/or a capture oligo sequence, thus in each reaction compartment, many assemblies can occur in parallel. In some embodiments, two or more copies of a barcode and/or a capture oligo sequence may be provided in one or more nucleic acid molecules on the bead. For example, a bead may comprise a clonal population of the same barcode and/or capture oligo sequences. According to one aspect, a plurality of beads are sequestered or contained within an emulsion droplet. According to one aspect, about 1 to about 5 beads are sequestered or contained within an emulsion droplet. According to one aspect, about 1 to about 2 beads are sequestered or contained within an emulsion droplet. According to one aspect, 1 bead or a single bead is sequestered or contained within an emulsion droplet.

The beads may be subject to temperature and reagents which remove or release the oligonucleotide sequences from the beads. For example, the beads may be incubated at a temperature which allows for a hybridized oligo to be released. The oligonucleotides are then contained within the emulsion droplet but are no longer attached to the beads. According to one aspect, the oligonucleotides are contained within the emulsion droplet along with reagents suitable for assembling the oligonucleotides into nucleic acids or a target nucleic acid.

### IV. Assembling Subsequences

In some embodiments, provided herein is a method of producing at least one target nucleic acid having a predefined sequence comprises providing at least a plurality of stem-loop oligonucleotides (hairpin oligos) comprising a 3′ single-stranded overhang, wherein the single-stranded 3′ overhang is capable of hybridizing (e.g., being complementary to) a sequence of a 3′ end region of another polynucleotide, e.g., a sequence of a single-stranded 3′ overhang of a double-stranded polynucleotide. Steps of synthesis can be repeated thereby generating the at least one target nucleic acid. In some embodiments, all steps are in a single reaction volume. In some embodiments, the overhang is between 3 and 20 nucleotides long. In some embodiments, the stem-loop oligonucleotide is at least 100 bps long. The stem-loop structure may be formed by designing the oligonucleotides to have complementary sequences within its single-stranded sequence whereby the single-strand folds back upon itself to form a double-stranded stem and a single-stranded loop. In some embodiments, the double-stranded stem domain can have at least about 10 base pairs and the single stranded loop has at least 3, at least 5, at least 10, at least 20, at least 50 nucleotides. The stem can comprise an overhanging single-stranded region, i.e., the stem is a partial duplex.

In some embodiments, the assembly of subsequences into an assembled product comprises concerted actions of one or more enzymes, including a ligase, a polymerase, and/or a Type IIS restriction enzyme.

A DNA ligase is an enzyme that catalyzes the formation of a phosphodiester linkage between the 5′ phosphorylated and 3′ hydroxylated ends of adjacent DNA nucleotides in dsDNA. The result is restored continuity to a DNA strand that previously harbored breaks. The value of this enzyme is clear from the process of DNA replication wherein ligation of discontinuous segments of DNA, Okazaki fragments, forms one continuous strand. DNA ligases vary in activity. Some enzymes can repair single-stranded nicks, and others play a role in fixing double-stranded breaks in DNA. Exemplary embodiments of DNA ligase include but are not limited to T4 DNA ligase, Taq DNA ligase, and DNA ligase (E. coli) Similar to the activity of DNA ligase, RNA ligase catalyzes the linkage of a 5′-phosphate terminus to a 3′-hydroxyl terminus. DNA and RNA ligase enzymes differ in their preferred substrate. RNA ligases have a greater affinity for RNA substrates and can use single-stranded RNA (ssRNA) and DNA-RNA hybrids as substrates. Exemplary embodiments of RNA ligases include but are not limited to T4 RNA ligase 1, T4 ligase 2, and TS2126 RNA ligase 1. In some embodiments, high fidelity ligases are useful in the methods of the present disclosure.

DNA polymerases catalyze the addition of a deoxyribonucleotide to a 3′ hydroxyl terminus attached to a template. Short strands of DNA or RNA nucleotides, primers, satisfy the requirement for a 3′ nucleotide terminus in a DNA duplex. Accordingly, DNA polymerase attaches the 5′ end of a new nucleotide to a 3′ of a primer. This results in polynucleotide synthesis in the 5′ to 3′ direction. Complementarity to base pairs in a template generally determines which nucleotides will be added by a DNA polymerase. Incorporation of the correct nucleotides to a growing strand of DNA, as determined by the template, is known as sequence fidelity. In an experiment where the results are heavily influenced by the DNA sequence, high fidelity DNA polymerase is of great value. Interestingly, there is wide variation in sequence fidelity among DNA polymerases. DNA polymerase can enhance sequence fidelity through the mechanisms of prevention, proofreading, and repair. Exemplary embodiments of DNA polymerase include but are not limited to Taq, Q5, and others. In some embodiments, high fidelity DNA polymerases are useful in the methods of the present disclosure.

Turning to the figures, FIG. 6A shows an exemplary method of using a support (e.g., bead or solid substrate) to capture polynucleotides for unidirectional assembly of a target polynucleotide. A single-stranded polynucleotide is directly or indirectly attached to a support. In some examples, the single-stranded polynucleotide comprises one or more useful moieties (e.g., sequences), e.g., an adapter, a tag, a primer binding moiety, a cleavage site, a UMI/UID, and/or a barcode, and does not comprise a subsequence to be assembled with other subsequences of a target sequence. In some examples, the single-stranded polynucleotide comprises one or more useful moieties (e.g., sequences) as well as a subsequence to be assembled with other subsequences of a target sequence. In Cycle 1 shown in FIG. 6A, the single-stranded polynucleotide is attached to a support, and a hairpin molecule comprises a 3′ overhang capable of hybridizing to a 3′ sequence of the single-stranded polynucleotide. A sequence in the hairpin molecule may be added to the single-stranded polynucleotide via hybridization, extension by a polymerase, and cleavage by a Type IIS restriction enzyme. These enzymes can be present during all steps of Cycle 1 and subsequent cycles (e.g., in a one-pot reaction), as explained in more detail elsewhere in the present disclosure. A ligase may also be present in the one-pot reaction, but is not necessary in Cycle 1 shown in FIG. 6A. In some embodiments, the nick in the hybridization complex shown in FIG. 6A is spaced from the 3′ end nucleotide of the hairpin molecule such that a polymerase is capable of extending the 3′ end of the single-stranded polynucleotide. In some embodiments, the nick is separated from the 3′ end nucleotide of the hairpin molecule by more than 5, more than 6, more than 7, more than 8, more than 9, more than 10, more than 11, more than 12, more than 13, more than 14, or more than 15 base pairs. In some embodiments, the 3′ overhang of the hairpin molecule is more than 5, more than 6, more than 7, more than 8, more than 9, more than 10, more than 11, more than 12, more than 13, more than 14, or more than 15 nucleotides in length. In some embodiments, the 3′ end nucleotide of the hairpin molecule may be blocked and/or not extended by a polymerase. In some embodiments, the 3′ end nucleotide of the hairpin molecule is extended by a polymerase using the single-stranded polynucleotide as a template.

FIG. 6B shows an exemplary method comprising Cycle 1 reactions where a single-stranded polynucleotide is not attached to a support (e.g., bead or solid substrate), and a hairpin molecule comprises a 3′ overhang capable of hybridizing to a 3′ sequence of the single-stranded polynucleotide. A sequence in the hairpin molecule may be added to the single-stranded polynucleotide via hybridization, extension by a polymerase, and cleavage by a Type IIS restriction enzyme, similar to the Cycle 1 reactions shown in FIG. 6A.

FIG. 6C and FIG. 6D show the first and second cycle, respectively, of an exemplary method of assembling a target polynucleotide. The first cycle as well as subsequent cycles of assembly can include individual steps of hybridization, ligation by a ligase, extension by a polymerase, and/or cleavage by a Type IIS restriction enzyme. These enzymes can be present during all steps of the cycle (e.g., in a one-pot reaction). In Cycle 1 shown in FIG. 6C, an oligo comprising a first subsequence to be incorporated into the target polynucleotide is attached to a support, and a second subsequence is contained in a second polynucleotide in the form of a hairpin molecule. In this configuration, the target polynucleotide is assembled in a unidirectional fashion extending away from the support.

In one embodiment, an oligo comprising the first subsequence has a free single-stranded 3′ end sequence but is otherwise double-stranded. In this embodiment, the free single-stranded 3′ end sequence hybridizes to the 3′ overhang of a hairpin molecule (e.g., as shown in step 1 of FIG. 6C). The hairpin molecule may contain the second subsequence, a Type IIS restriction enzyme recognition sequence, a tag sequence, and a blocked 5′ end. In some embodiments, even in the presence of a polymerase, neither strand of the hybridization complex can be extended due to the close proximity of the nicks in each strand, which nicks resemble a double stranded break (DSB). In some embodiments, the nicks can be separated from each other by 1, 2, 3, 4, 5, 6, 7, 8, 9, 10, 11, 12, 13, 14, or 15 or more base pairs. In some embodiments, the polymerase is not a Nonhomologous end-joining (NHEJ) polymerase, e.g., a polymerase that is able to fill in break termini containing 3′ overhangs that lack a primer strand. In addition, the restriction enzyme recognition sequence in the hairpin molecule cannot be cleaved by the Type IIS restriction enzyme because the restriction enzyme recognition sequence is single-stranded. Thus, after hybridization, only the ligase is capable of acting on the hybridized complex and ligates the 3′ end of the hairpin molecule to the first subsequence (e.g., as shown in step 2 of FIG. 6C). The 3′ end of the first subsequence is not ligated to the blocked 5′ end of the hairpin molecule.

In some embodiments, the oligo comprising the first subsequence is single-stranded, and the 3′ overhang of the hairpin molecule can be of a length such that after hybridization (e.g., as shown in step 1′ of FIG. 6C), there is not a second nick in close proximity to the nick at the 3′ end of the first subsequence, and a polymerase (e.g., one that is not an NHEJ polymerase) is capable of extending the 3′ end of the first subsequence using the hairpin addition oligo as a template. Thus, in some examples, ligation is not necessary to enable extension by the polymerase.

In some embodiments, extension by the polymerase occurs beginning at the 3′ end of the first subsequence (e.g., as shown in step 3 of FIG. 6C). The polymerase may displace the strand having the complementary sequence and “unfold” the stem region of the hairpin molecule, e.g., thereby linearizing the second polynucleotide and allowing the polymerase to use the second polynucleotide as a template for extension. In some embodiments, the polymerase may have a 5′ to 3′ exonuclease activity, which can be coupled to the polymerization activity to displace DNA strands. In some embodiments, primer extension by the polymerase results in a double-stranded polynucleotide containing the first subsequence, the second subsequence, the Type IIS restriction enzyme recognition sequence, the tag sequence, and the 5′ end sequence of the second polynucleotide. After primer extension, the Type IIS restriction enzyme recognition sequence is double-stranded and can be cleaved by the Type IIS restriction enzyme (e.g., as shown in step 4 of FIG. 6C). In some embodiments, this cleavage removes the tag sequences and the 5′ end sequences of the second polynucleotide. In some embodiments, cleavage is asymmetric across strands and produces a single-stranded 3′ end sequence in the second subsequence, thereby allowing for additional cycles of assembly.

As shown in FIG. 6D, the Cycle 2 assembly proceeds in a similar fashion to that described for Cycle 1. In this cycle, another hairpin molecule containing a third subsequence and a 3′ overhang complementary to the single-stranded 3′ end sequence of the second subsequence generated in Cycle 1 is provided. This hairpin molecule can be present during cycle 1 (e.g., as in a one-pot reaction) but would not have been able to hybridize prior to the sequence complementary to its 3′ overhang being made available via cleavage of the double-stranded polynucleotide generated in Cycle 1. After hybridization, ligation, extension, and cleavage, a double-stranded polynucleotide containing the first, second, and third subsequences is produced, with the third subsequence containing a single-stranded 3′ end sequence. Additional hairpin molecules each containing a 4^{th}, 5^{th}, . . . , and n^{th} subsequences can be added in serial in a predetermined order.

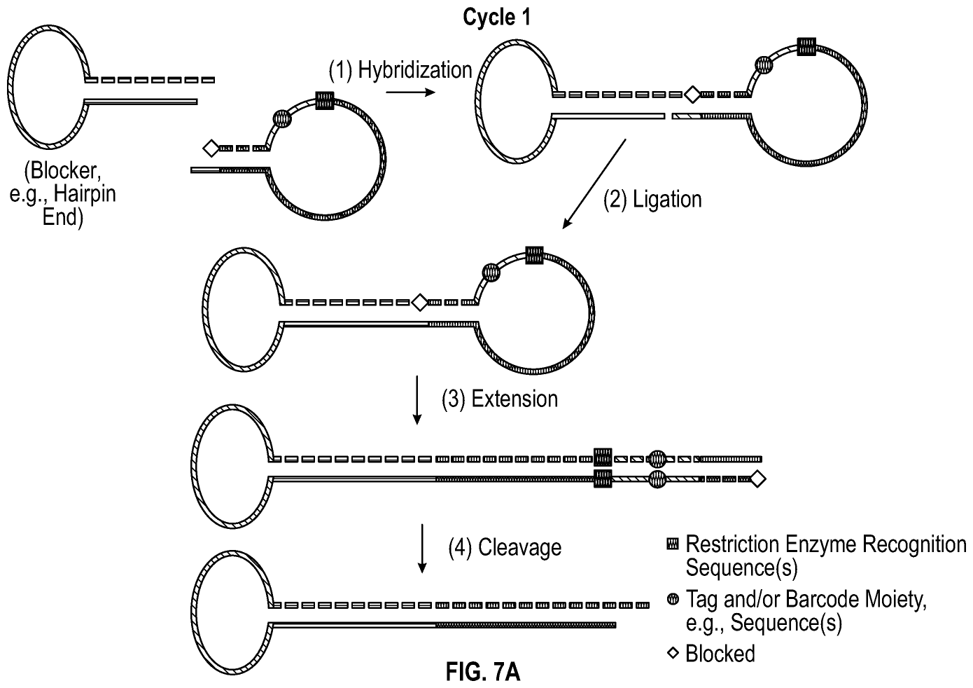

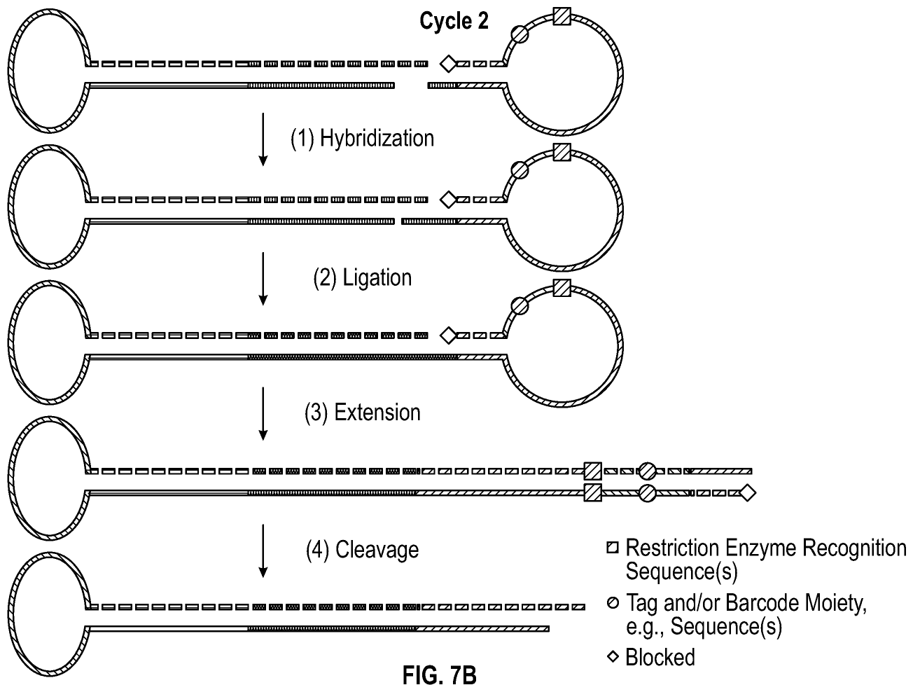

FIG. 7A and FIG. 7B show the first and second cycle, respectively, of an exemplary method of assembling a target polynucleotide. In some examples, the first subsequence with a single-stranded 3′ end sequence is incorporated into a polynucleotide containing a blocker, e.g., a hairpin end, and a second subsequence is contained in a second polynucleotide in the form of a hairpin molecule. In this manner, the target polynucleotide is assembled in a unidirectional fashion extending away from the blocker. In some embodiments, assembly proceeds as described for FIG. 6C and FIG. 6D, and the hairpin blocker may but does not have to be immobilized. For example, the reactions may occur in a homogenous format, e.g., in a solution.

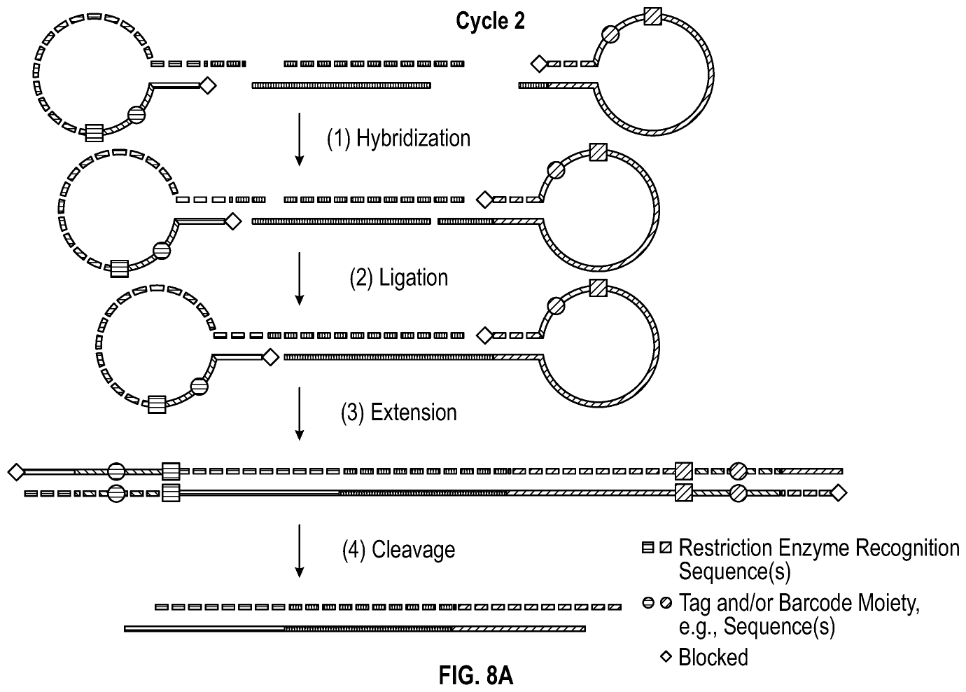

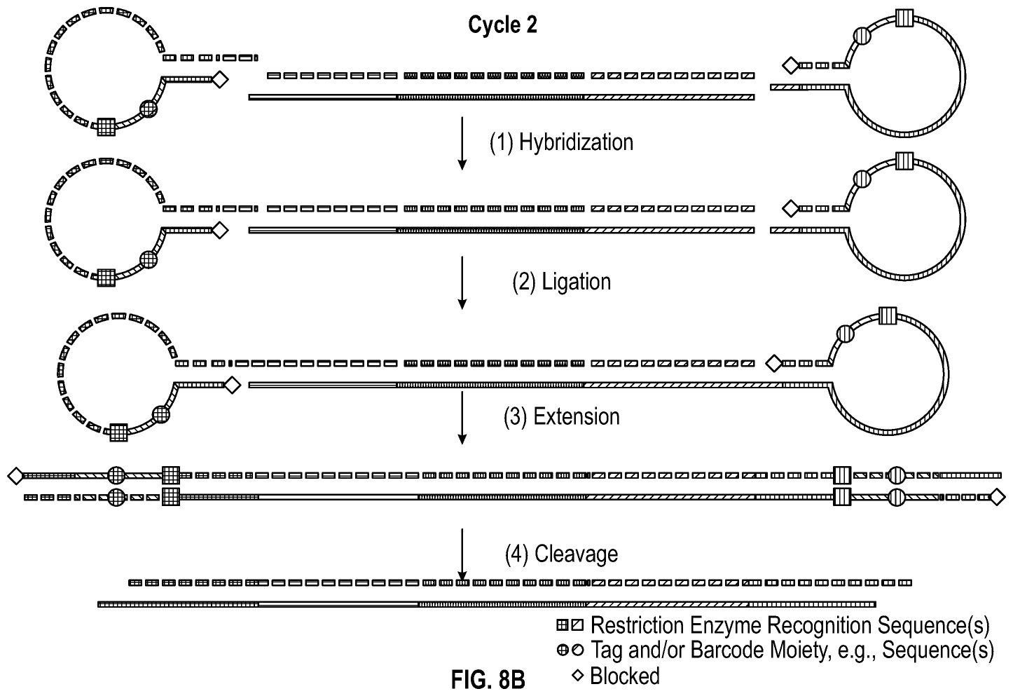

FIG. 8A and FIG. 8B show the first and second cycle, respectively, of an exemplary method of assembling a target polynucleotide. In this exemplary method, the target polynucleotide is assembled in a bidirectional fashion, i.e., from both ends of a linear first polynucleotide. In some examples, the first polynucleotide includes two single-stranded 3′ end sequences and a first subsequence to be included in the target polynucleotide. Additional subsequences to be incorporated into the target polynucleotide are contained in hairpin molecules, e.g., as shown FIG. 4A and FIG. 4B.

As shown in FIG. 8A, a first hairpin molecule contains a 3′ overhang complementary to one of the single-stranded 3′ end sequences of the linear polynucleotide, and a second hairpin molecule contains a 3′ overhang complementary to the other single-stranded 3′ end sequence of the linear polynucleotide. The subsequence of each hairpin molecule is incorporated into a target sequence, similar to the process described in FIG. 6C and FIG. 6D. After hybridization (e.g., as shown in step 1 of FIG. 8A), the 3′ ends of the hairpin molecules are ligated to the linear polynucleotide (e.g., as shown in step 2 of FIG. 8A), while the 5′ ends of the hairpins remain blocked and are not ligated. In some embodiments, prior to the ligation, the nicks on the two strands are in proximity to each other and resemble a DSB; for example, the nicks can be separated from each other by 1, 2, 3, 4, 5, 6, 7, 8, 9, 10, 11, 12, 13, 14, or 15 or more base pairs. In some examples, although a polymerase is present in a reaction volume (e.g., an emulsion droplet), the polymerase does not extend the 3′ end(s) of the first linear polynucleotide until the nick on the opposite strand is ligated by a ligase.

After ligation, both hairpins are linearized during extension and used as templates (e.g., as shown in step 3 of FIG. 8A), in this manner producing a double-stranded polynucleotide containing the subsequence of the first hairpin molecule, the subsequence of the linear polypeptide, and the subsequence of the second hairpin molecule. On each side of the double-stranded polynucleotide is a double-stranded restriction enzyme recognition sequence. These restriction enzyme recognition sequences are cleaved by a Type IIS restriction enzyme (e.g., as shown in step 4 of FIG. 8A), producing a single-stranded 3′ end sequence on each side of the double-stranded polynucleotide. It should be noted that the Type IIS restriction enzyme recognition sequences in the first and second hairpin molecules can be the same or different, and the single-stranded 3′ end sequences on each side of the double-stranded polynucleotide can be the same or different.

As shown in FIG. 8B, the second cycle of assembly proceeds in a manner similar to that described for FIG. 6D. In this cycle, additional hairpin molecules are provided, the hairpin molecules containing 3′ overhangs complementary to either single-stranded 3′ end sequence of the double-stranded polynucleotide. These hairpin molecules can be present during Cycle 1 (e.g., as in a one-pot reaction) but would not have been able to hybridize prior to sequences complementary to their 3′ overhangs being made available via cleavage of the double-stranded polynucleotide. After hybridization, ligation, extension, and cleavage, a double-stranded polynucleotide containing five subsequences is produced, with each end containing a single-stranded 3′ end sequence. Additional hairpin molecules containing subsequences can be added in serial in a predetermined order.

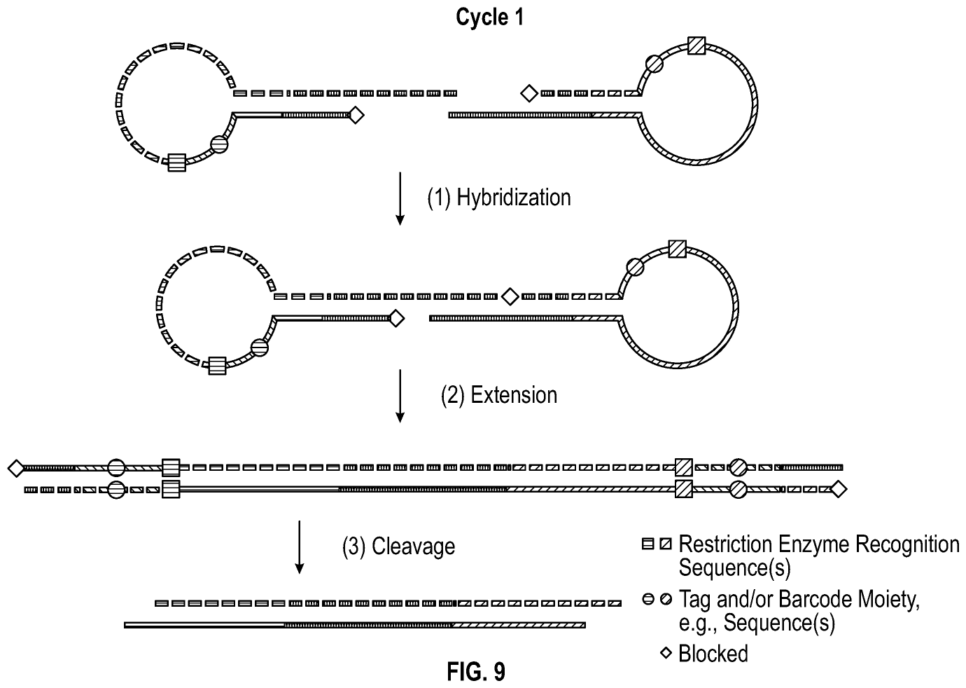

FIG. 9 shows the first cycle of an exemplary method of assembling a target polynucleotide. In some examples, assembly proceeds in a bidirectional manner but without the initial inclusion of a linear polypeptide. Instead, each of the hairpin oligos includes a longer single-stranded 3′ end sequence. Portions or all of the single-stranded 3′ end sequences of the hairpin molecules are complementary to one another. Upon hybridization (e.g., as shown in step 1 of FIG. 9), extension using the hairpin molecules as templates is possible without ligation, as the nicks are not close enough to one another to interfere with polymerase activity. After extension (e.g., as shown in step 2 of FIG. 9) and cleavage (e.g., as shown in step 3 of FIG. 9), a double-stranded polynucleotide containing the subsequences of the hairpin molecules is produced, with each end containing a single-stranded 3′ end sequence. Cycle 2 and subsequent cycles of assembly can proceed essentially as described for FIG. 6D.

In some embodiments, the emulsion, and therefore the beads within the emulsion droplets, is thermal-cycled to assemble the oligonucleotides, such as double stranded DNA in each emulsion into nucleic acids, such as target nucleic acids, such as full length fragments.

In some embodiments, in order to assemble the oligonucleotides, the emulsion does not need to be thermal-cycled. In some embodiments, one or more reactions during the assembly of the oligonucleotides is an isothermal reaction. In some embodiments, the methods disclosed herein allow for the joining of multiple nucleic acid fragments in an isothermal process, e.g., a process at about 10° C., at about 15° C., at about 20° C., at about 25° C., at about 30° C., at about 35° C., at about 40° C., at about 45° C., at about 50° C., at about 55° C., at about 60° C., at about 65° C., at about 70° C., at about 75° C., at about 80° C., or any range between the foregoing.

In some embodiments, the isothermal process comprises hybridization, ligation, primer extension, and/or Type IIS restriction enzyme cleavage. In some embodiments, the isothermal process comprises repeated cycles of hybridization, ligation, primer extension, and/or Type IIS restriction enzyme cleavage.

In some embodiments, the emulsion is then de-emulsified, and nucleic acids can be pooled, partitioned, and/or processed, e.g., for the next level assembly or for downstream analysis or application.

In some embodiments, the nucleic acids can be separated such as by gel purification or other methods known to those of skill in the art. According to one aspect, nucleic acids can be separated and correctly assembled products of desired length can be isolated and recovered using standard gel electrophoresis techniques known to those of skill in the art. Accordingly, a library of specifically assembled sequences is constructed, which can be further isolated by PCR if necessary, or used directly as a library in other cases.

### V. Multiplexed and/or Serial Subsequence Assembly

In some embodiments, a plurality of oligonucleotides can be assembled in parallel into a single or a plurality of desired polynucleotide constructs using the methods described herein. In some embodiments, the assembly procedure may include several parallel and/or sequential reaction steps in which a plurality of different nucleic acids or oligonucleotides are immobilized, partitioned, and are combined (e.g., released into a partition) in order to be assembled to generate a longer nucleic acid product to be used for further assembly, cloning, or other applications.

In certain exemplary embodiments, methods are provided for synthesizing between about 1 to about 100,000 target nucleic acid sequences, between about 1 to about 75,000 target nucleic acid sequences, between about 1 to about 50,000 target nucleic acid sequences, between about 1 to about 10,000 target nucleic acid sequences, between about 100 to about 5,000 target nucleic acid sequences, between about 500 to about 1,000 target nucleic acid sequences or any range or value in between whether overlapping or not. According to certain aspects, methods are provided for simultaneously synthesizing between about 1 to about 10,000 target nucleic acid sequences, between about 100 to about 5,000 target nucleic acid sequences, between about 500 to about 1,000 target nucleic acid sequences or any range or value in between whether overlapping or not. The synthesis of a plurality of target nucleic acids describe herein is considered simultaneous to the extent that a plurality of emulsion droplets are created with each droplet within the plurality of droplets having an oligonucleotide set therein under conditions and with reagents capable of synthesizing a target nucleic acid sequence. Accordingly, each emulsion droplet is considered a discrete reaction volume within which a target nucleic acid sequence is synthesized. Accordingly, methods of the present disclosure include synthesizing between about 1 and about 10,000 target nucleic acids having lengths between about 300 to about 10,000 nucleotides, for example, between about 300 and about 5,000 nucleotides, or between about 1,000 and about 5,000 nucleotides. Still accordingly, methods of the present disclosure include synthesizing within emulsion droplets between about 1 and about 10,000 target nucleic acids having lengths between about 300 to about 5,000 nucleotides. According to a certain aspect, one target nucleic acid is synthesized within a single emulsion droplet. According to a certain aspect, a plurality of target nucleic acids are synthesized simultaneously within an emulsion where a target nucleic acid is synthesized in each of a plurality of emulsion droplets.

Also provided herein are method comprising consecutive levels of assembly, e.g., assembling all or a subset of assembled products from a previous level of assembly into even longer products.

FIG. 10 shows an exemplary method comprising consecutive levels of assembly using sequential addition of hairpin oligos. In this example, the 5′ end of Oligo 1 is blocked from ligation, and subsequent oligos up until Oligo N-1 are also blocked at their 5′ ends (e.g., due to dephosphorylation). After assembly of subsequence N-1 into the growing double-stranded product, Oligo N (optionally comprising subsequence N) hybridizes to the product. Because Oligo N is not blocked at its 5′ end, a ligase in the emulsion droplet ligates the 3′ end of the overhang of the double-stranded product to the 5′ end of Oligo N, as well as the 3′ end of Oligo N to the recessed 5′ end of the double-stranded product. Thus, a polymerase in the emulsion droplet is not able to extend the 3′ end overhang of the double-stranded product as in previous cycles of oligo addition. The product is a hairpin molecule that resembles the hairpin addition oligos (e.g., having a 3′ end overhang, a blocked 5′ end, a stem region, and a loop region comprising a Type IIS restriction enzyme recognition sequence and a useful sequence, such as a capture tag sequence) but is much longer. The product can be used as building blocks in a higher level assembly, employing the sequential addition of hairpin molecules disclosed herein and/or one or more other methods of assembly.

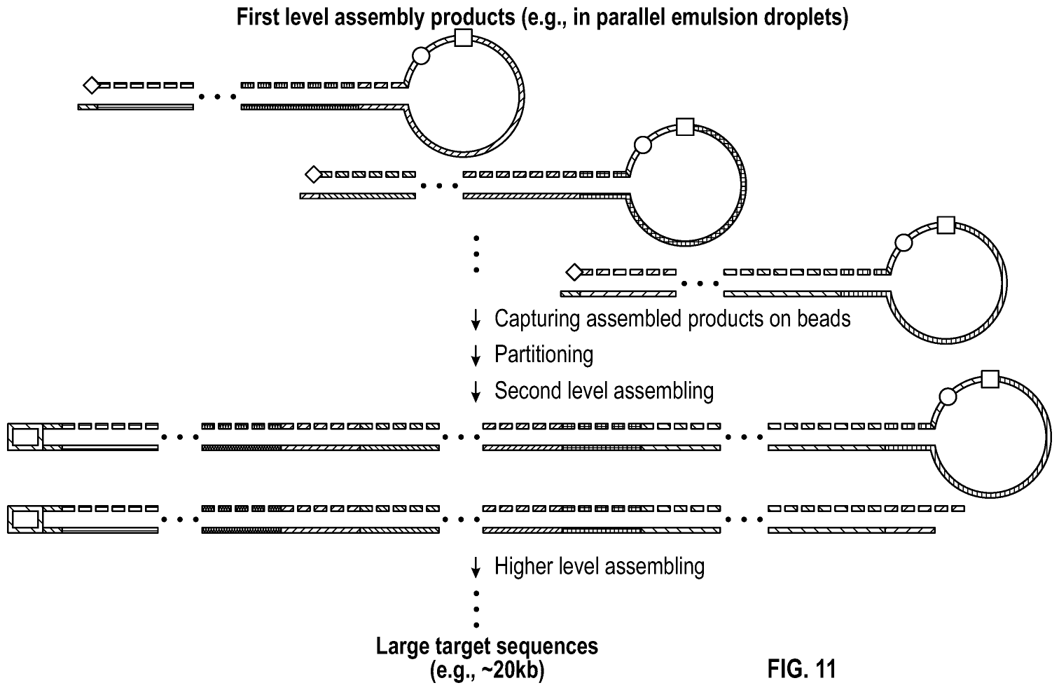

FIG. 11 shows an exemplary method comprising a first level and a second level of assembly and optionally even higher levels of assembly. Hairpin products of a first level assembly process may be generated in parallel from emulsion droplets, e.g., as shown in FIG. 10. The emulsion is broken and the products are pooled. The hairpin products may comprise a plurality of subsets, and products in each subset can be designed such that they are added sequentially in a predetermined order to form a growing assembly product. A subset of hairpin products of the plurality of subsets may be captured on a bead by virtue of the bead comprising one or more capture oligos complementary to one or more capture tag sequences of the hairpin products of the subset. The beads having captured hairpin products are then partitioned into emulsion droplets, the hairpin products of the same subset are released in a emulsion droplet, and a second level assembly is carried out essentially as described for the first level assembly. Products of the second level assembly may comprise a hairpin end (e.g., for a third level assembly using sequential addition of hairpin molecules) or other types of end, e.g., a sticky end, a blunt end, an end having an overlapping sequence with other sequences, an end having an adapter sequence, and/or an end immobilized on a support.

By way of example, a first level assembly may generate 1,000 different assembled sequences. Each sequence is assembled in an emulsion droplet comprising the oligos that comprise subsequences of the assembled sequence. Oligos in each droplet are captured onto a bead by virtual of having a common level 1 capture tag sequence (e.g., barcode) unique to the oligos. In other words, a bead library comprising capture oligos for the 1,000 different level 1 barcodes may be used to pull down and partition the oligos. The seed oligos and/or terminal oligos for assembling level 1 assembled sequences 1-10, 11-20, 21-30, . . . , 981-990, and 991-1,000 share a common level 2 capture tag sequence (e.g., barcode) T1 to T100, respectively. In some embodiments, T1-T100 are provided in the single-stranded loop of the terminal oligo for assembling a level 1 assembled sequence, e.g., as shown in FIG. 10 and FIG. 11. For instance, T1 is shared by and specific to all level 1 assembled sequences 1-10, T2 is shared by and specific to all level 1 assembled sequences 11-20, etc. Thus, level 1 assembled sequences 1-10, 11-20, 21-30, . . . , 981-990, and 991-1,000 can be pooled following the level 1 assembly reactions and captured onto beads each comprising a level 2 capture oligo that specifically hybridizes to one of T1-T100. In this way, level 2 assembly reactions each assembling 10 level 1 assembled sequences can be performed in parallel, generating 100 different level 2 assembled sequences. Even higher level assembly may be performed similarly, using sequential hairpin oligo addition and/or other assembly methods disclosed herein.

In some embodiments, the next tier or higher level assembly comprises one or more other assembly reactions, such as an in vitro or in vivo assembly reaction. For instance, a higher level assembly may comprise a polymerase cycle assembly (PCA, also known as assembly PCR) (e.g., using a DNA polymerase), SLIC (sequence- and ligation-independent cloning) (e.g., using a T4 DNA polymerase), Golden Gate assembly (e.g., using adapters on both ends of a double-stranded DNA fragments), Gibson assembly (e.g., Gibson et al., Nature Methods 6:343-345 (2009), e.g., using a T5 exonuclease, a DNA polymerase, and a Taq ligase), an in vivo (e.g., in yeast) assembly using oligonucleotide with overlaps, and/or a transformation-associated recombination. Exemplary assembly methods are reviewed in Zhang et al. (2020) Annu. Rev. Biochem. 89: 77-101, which is incorporated herein by reference in its entirety.

In some embodiments, the methods comprise assembling products from a lower level assembly using sequential addition of hairpin oligos disclosed herein. In some embodiments, hairpin oligos are designed and generated from products of the lower level assembly. The lower level assembly may comprise one or more other assembly reactions, such as an in vitro or in vivo assembly reaction, e.g., PCA, SLIC, Golden Gate assembly, Gibson assembly, an in vivo assembly using oligonucleotide with overlaps, and/or a transformation-associated recombination.

In certain exemplary embodiments, the method disclosed herein comprise using assembly PCR (PCA) to produce a nucleic acid sequence from a plurality of oligonucleotide sequences that are members of a particular oligonucleotide set. “Assembly PCR” refers to the synthesis of long, double stranded nucleic acid sequences by performing PCR on a pool of oligonucleotides having overlapping segments. Assembly PCR is discussed further in Stemmer et al. (1995) Gene 164:49. In certain aspects, PCR assembly is used to assemble single stranded nucleic acid sequences (e.g., ssDNA) into a nucleic acid sequence of interest. In other aspects, PCR assembly is used to assemble double stranded nucleic acid sequences (e.g., dsDNA) into a nucleic acid sequence of interest. Assembly PCR, as well as any other suitable in vitro or in vivo assembly reactions, may be used in any step of any level of assembly disclosed herein.

### VI. Processing, Analyzing, and/or Selecting Assembled Sequences

Also provided herein are methods and compositions for the processing, analysis, and/or selection of one or more assembled sequences.

In some embodiments, it is desirable to remove one or more moieties (e.g., sequences) from an assembled product, for example, to process the assembled product for a next level assembly process, and/or for a downstream analysis or application, e.g., for transfecting or transforming a cell using the assembled product.

In some embodiments, it is desirable to remove one or more sequences from an assembled product, and the sequence(s) to be removed may be contributed by a seed oligo, an addition oligo, and/or a terminal oligo. In particular embodiments, one or more sequences from a seed oligo are removed from the assembled product. These sequences may comprises one or more useful sequences disclosed herein, e.g., in Section II-B-d, such as a primer binding sequence or a barcode sequence. In particular embodiments, a restriction enzyme recognition site can be present within the seed oligo, and a restriction enzyme can be used to cleave the assembled product at or near the restriction enzyme recognition site thereby separating a sequence to be removed from the remaining assembled product sequence. In particular embodiments, one or more uracil residues may be introduced into the seed oligo and/or an assembled product comprising a sequence from the seed oligo, and a USER (Uracil-Specific Excision Reagent) enzyme can be used to nick and/or cleave the assembled product, thereby separating a sequence to be removed from the remaining assembled product sequence. In some embodiments, all of the seed oligo sequence is part of the desired assembled product sequence, and no remove of a seed oligo sequence is needed.

In particular embodiments, one or more sequences from a terminal oligo are removed from the assembled product. These sequences may comprises one or more useful sequences disclosed herein, e.g., in Section II-B-d, such as a primer binding sequence or a barcode sequence. In particular embodiments, a restriction enzyme recognition site can be present within the terminal oligo (e.g., in the double-stranded stem region of a hairpin oligo), and a restriction enzyme can be used to cleave the assembled product at or near the restriction enzyme recognition site thereby separating a sequence to be removed from the remaining assembled product sequence. In particular embodiments, one or more uracil (U) residues may be introduced into the terminal oligo and/or an assembled product (e.g., Us in a single-stranded loop region of a hairpin oligo), and a USER enzyme then nicks the single-stranded loop region in the assembled product, thereby separating a sequence to be removed from the remaining assembled product. In some embodiments, processing the hairpin loop region is not necessary, e.g., for using the assembled product in a next level assembly, e.g., as shown in FIG. 10 and FIG. 11.

In some embodiments, an assembled product (e.g., a full length target nucleic acid to be produced or any intermediate thereof during assembly) can include primer binding sequences, so that the assembled product can be amplified, e.g. using PCR primers. The primer binding sequences can be located at one or both ends of the assembled product, for example, one provided by a seed oligo and another provided by a terminal oligo. In some embodiments, one or more of the primer binding sequences can be provided by a seed oligo, an addition oligo, and/or a terminal oligo, for example, one provided by a seed oligo and another provided by an addition oligo (e.g., as a sequence of the target nucleic acid sequence such as a sequence spanning the junction of two subsequences provided in separate addition oligos during assembly). In other examples, one primer binding sequence is provided by an internal addition oligo (e.g., as a sequence of the target nucleic acid sequence such as a sequence spanning the junction of two subsequences provided in separate addition oligos during assembly) and another primer binding sequence is provided in the terminal oligo, which may or may not comprise a subsequence of the target nucleic acid sequence. In some embodiments, one or more of the primer binding sequences can be different from a sequence of the target nucleic acid sequence. In some embodiments, one or more of the primer binding sequences can be a sequence of the target nucleic acid sequence.

In some embodiments, the primer sequences and primer binding sequences can be designed to facilitate amplification of long products, e.g., of about 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb, 17 kb, 18 kb, 19 kb, 20 kb, 21 kb, 22 kb, 23 kb, 24 kb, 25 kb, 26 kb, 27 kb, 28 kb, 29 kb, 30 kb, 31 kb, 32 kb, 33 kb, 34 kb, 35 kb, 36 kb, 37 kb, 38 kb, 39 kb, 40 kb, 41 kb, 42 kb, 43 kb, 44 kb, 45 kb, 46 kb, 47 kb, 48 kb, 49 kb, 50 kb, or longer, or in a range between any of the foregoing sizes. In some embodiments, an assembled product is amplified using a long range PCR reaction, and the PCR primers and primer binding sequences and other conditions are designed for such long range PCR.

In some embodiments, the primer Tₘ is a low Tₘ, e.g., at or at about 50° C., at or at about 45° C., at or at about 40° C., or lower than 40° C., or in a range between any of the foregoing. In some embodiments, the PCR reaction is performed using an optimal annealing temperatures (Tₐ), e.g., the value for the primer with the lowest Tₘ (Tₘ^{min}):

Tₐ(° C.)=Tₘ^{min}+ln L.

where L is length of the PCR product. In some embodiments, the PCR reaction is performed at a high Tₐ, e.g., at or at about 50° C., at or at about 55° C., at or at about 60° C., at or at about 65° C., at or at about 70° C., or higher than 70° C., or in a range between any of the foregoing.

In some embodiments, an assembled product (e.g., a full length target nucleic acid to be produced or any intermediate thereof during assembly) can be separated such as by gel purification or other methods known to those of skill in the art. According to one aspect, nucleic acids can be separated and correctly assembled products of desired length can be isolated and recovered using standard gel electrophoresis techniques known to those of skill in the art. Accordingly, a library of specifically assembled sequences is constructed, which can be further isolated by PCR if necessary, or used directly as a library in other cases.

Errors may be introduced into an assembled product, including errors due to polymerase activity, oligo synthesis, and/or errors during assembly of oligos. Thus, provided herein are methods for analyzing the sequence of an assembled product, selecting assembled molecules of the correct sequence, and/or correcting errors in assembled molecules. In certain embodiments, these method comprises amplification of an assembled product, e.g., using PCR, and/or determining a sequence of an assembled product, e.g., using a direct sequencing or an indirect sequencing method.

In certain embodiments, methods of determining the sequence of one or more nucleic acid sequences of interest are provided. Sequencing methods include, but are not limited to, Maxam-Gilbert sequencing-based techniques, chain-termination-based techniques, shotgun sequencing, bridge PCR sequencing, single-molecule real-time sequencing, ion semiconductor sequencing (Ion Torrent sequencing), nanopore sequencing, pyrosequencing (454), sequencing by synthesis, sequencing by ligation (SOLiD sequencing), sequencing by electron microscopy, dideoxy sequencing reactions (Sanger method), massively parallel sequencing, polony sequencing, and DNA nanoball sequencing. High-throughput sequencing methods, e.g., on cyclic array sequencing using platforms such as Roche 454, Illumina Solexa, AB-SOLiD, Helicos, Polonator platforms and the like, can also be utilized. Exemplary high-throughput sequencing methods are described in U.S. Ser. No. 61/162,913, filed Mar. 24, 2009. In certain embodiments, a Next Generation Sequencing (NGS) method is used, e.g., sequencing methods that allow for massively parallel sequencing of clonally amplified and of single nucleic acid molecules during which a plurality, e.g., millions, of nucleic acid fragments from a single sample or from multiple different samples are sequenced in unison. Non-limiting examples of NGS include sequencing-by-synthesis, sequencing-by-ligation, real-time sequencing, and nanopore sequencing.

Contiguous sequences may be derived from an individual sequence read, including either short or long read-length sequencing. Long read-length sequencing technologies include, for example, single molecule sequencing, such as SMRT Sequencing and nanopore sequencing technologies. See, e.g., Koren et al., One chromosome, one contig: Complete microbial genomes from long-read sequencing and assembly, Curr. Opin. Microbiol., vol. 23, pp. 110-120 (2014); and Branton et al., The potential and challenges of nanopore sequencing, Nat. Biotechnol., vol. 26, pp. 1146-1153 (2008). Contiguous sequences may also be derived from assembly of sequence reads that are aligned and assembled based upon overlapping sequences within the reads. When using multiple sequence reads, phasing can be determine by physically partitioning the originating molecular structures or by using other known linkage data, e.g., the tagging with molecular barcodes (e.g., UMIs or UIDs). Methods and compositions of using UMIs or UIDs are described, e.g., in U.S. Pat. Nos. 9,085,798 and 9,476,095, incorporated herein by reference. The overlapping sequence reads may include short reads, e.g., less than 500 bases, such as, in some cases from approximately 100 to 500 bases, and in some cases from 100 to 250 bases, or based upon longer sequence reads, e.g., greater than 500 bases, 1000 bases or even greater than 10,000 bases. The short reads are phased by using, for example, 10× or Illumina synthetic long read molecular phasing technology.

In some embodiments, an assembled product comprises one or more unique molecular identifier (UMI) sequences, which may be used to identify products having the correct target sequences. In some embodiments, one or more primers that are complementary or capable of hybridizing to the one or more UMI sequences are used to amplify and/or select products having the correct target sequences. In some embodiments, one or more capture oligos (e.g., on a bead) that are complementary or capable of hybridizing to the one or more UMI sequences are used to capture and/or select products having the correct target sequences. In some embodiments, the one or more UMI sequences are complementary or capable of hybridizing to both the one or more primers and the one or more capture oligos.

In some embodiments, products having the correct target sequences may be identified and/or selected for using an in vitro method and/or an in vivo method.

In some embodiments, products having the correct target sequences are identified and/or selected for by one or more primers and/or probes that are complementary or capable of hybridizing to one or more sequences that span the junction of two consecutive subsequences in a correctly assembled target sequence. In some embodiments, one or more capture oligos (e.g., on a bead) that are complementary or capable of hybridizing one or more sequences that span the junction of two consecutive subsequences in a correctly assembled target sequence are used to capture and/or select molecules having the correct target sequences.

In some embodiments, assembled products are introduced into a population of viruses or cells, and molecules having the correct target sequences may be identified and/or selected for by analyzing a viral or cell phenotype. In some embodiments, the assembled products comprise linear molecules (e.g., as shown in FIG. 4A) and/or circular molecules (e.g., as shown in FIG. 4B). In some embodiments, the linear molecules and/or circular molecules are introduced into a population of viruses or cells, e.g., to transfect or transform a cell. In some embodiments, viruses and/or cells comprising only one assembled molecule per virus or cell can be identified and/or selected from further analysis. For example, a correctly assembled sequence may comprise a marker, e.g., a sequence that can be expressed by a virus or cell to lead to a detectable change in a phenotype, e.g., a change from the presence of a phenotype to the absence of the phenotype or vice versa, or a change of a detectable signal in magnitude, duration, or other spatial and/or temporal characteristics. The population of viruses or cells can be analyzed so that individual clones or cells containing a correctly assembled target sequence may be identified, for example, using a single cell analysis. Technologies such as fluorescence-activated cell sorting (FACS) allow the precise isolation of selected single cells from complex samples, while high throughput single cell partitioning technologies, enable the simultaneous molecular analysis of hundreds or thousands of single unsorted cells. Exemplary methods for single cell isolation include: dielectrophoretic digital sorting, enzymatic digestion, FACS, hydrodynamic traps, laser capture microdissection, manual picking, microfluidics, micromanipulation, serial dilution, and Raman tweezers.

In certain exemplary embodiments, various error correction methods are provided to remove errors in oligonucleotide sequences, subassemblies and/or nucleic acid sequences of interest. The term “error correction” refers to a process by which a sequence error in a nucleic acid molecule is corrected (e.g., an incorrect nucleotide at a particular location is changed to the nucleic acid that should be present based on the predetermined sequence). Methods for error correction include, for example, homologous recombination or sequence correction using DNA repair proteins.

The term “DNA repair enzyme” refers to one or more enzymes that correct errors in nucleic acid structure and sequence, i.e., recognizes, binds and corrects abnormal base-pairing in a nucleic acid duplex. Examples of DNA repair enzymes include, but are not limited to, proteins such as mutH, mutL, mutM, mutS, mutY, dam, thymidine DNA glycosylase (TDG), uracil DNA glycosylase, AlkA, MLH1, MSH2, MSH3, MSH6, Exonuclease I, T4 endonuclease V, Exonuclease V, RecJ exonuclease, FEN1 (RAD27), dnaQ (mutD), polC (dnaE), or combinations thereof, as well as homologs, orthologs, paralogs, variants, or fragments of the forgoing. In certain exemplary embodiments, the ErrASE system is used for error correction (Novici Biotech, Vacaville, Calif.). Enzymatic systems capable of recognition and correction of base pairing errors within the DNA helix have been demonstrated in bacteria, fungi and mammalian cells and the like.

According to one aspect, nucleic acids made according to the methods described herein can be error corrected by the formation of hetero-duplexes in the emulsion using techniques known to those of skill in the art and described herein such as MutS-based, resolvase-based, ErrASE-based and the like. Exemplary methods include those described in Can et al., Nucl. Acids Res., 32(20):e162 (2004) and Saaem et al., Nucl. Acids Res., doi: 10.1093/nar/gkr887 (2011) each of which are hereby incorporated by reference in their entireties.

### VII. Compositions and Kits

Provided are compositions and kits, for example, comprising one or more polynucleotides disclosed herein for performing the methods provided herein, for example, reagents required for one or more steps including designing of oligos, oligo capturing and partitioning, hybridization, ligation, primer extension, restriction enzyme digestion, amplification, detection, sequencing, selecting correctly assembled sequences, and/or sample preparation.

In some aspects, provided herein are compositions, including molecules, complexes, conjugates, and products and intermediates of any method disclosed herein, including those described in words and/or in the drawings. Kits comprising these compositions, optionally with instruction to use, are also encompassed in the present disclosure.

In some aspects, provided herein is a pool of polynucleotides comprising polynucleotide sets P11, . . . , and P1j₁; . . . ; Pk1, . . . , and Pkjₖ; . . . ; and Pi1, . . . , and Pijᵢ, wherein i, j₁, . . . , jₖ, . . . , jᵢ, and k are integers, i, j₁, . . . , jₖ, . . . , and jᵢ are independently 2 or greater, and 1≤k≤i, wherein Pk1, . . . , and Pkjₖ comprise subsequences Sk1, . . . , and Skjₖ, respectively, which form target sequence S′k, wherein at least one of Pk1, . . . , and Pkjₖ comprises, in the 3′ to 5′ direction: (i) a single-stranded 3′ end sequence, (ii) the subsequence of target sequence S′k, (iii) a Type IIS restriction enzyme recognition sequence, and (iv) a complementary sequence capable of hybridizing to all or a portion of the subsequence of target sequence S′k, wherein the at least one of Pk1, . . . , and Pkjₖ further comprises a tag Tk in all or a subset of Pk1, . . . , and Pkjₖ, wherein the at least one of Pk1, . . . , and Pkjₖ is capable of forming a hairpin molecule comprising a 3′ overhang, a stem formed by intramolecular nucleotide base pairing between all or a portion of the subsequence of target sequence S′k and the complementary sequence, and a loop, and wherein the hairpin molecule is in a configuration that is not cleaved by a Type IIS restriction enzyme.

The various components of the kit may be present in separate containers or certain compatible components may be precombined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents, such as enzymes and buffers for oligo capturing and partitioning, hybridization, ligation, primer extension, restriction enzyme digestion, amplification, detection, sequencing, selecting correctly assembled sequences, and/or sample preparation, such as ligases, polymerases, and/or Type IIS enzymes. In some aspects, the kit can also include any of the reagents described herein, e.g., wash buffer, and ligation buffer. In some embodiments, the kits contain reagents for detection and/or sequencing. In some embodiments, the kits optionally contain other components, for example: nucleic acid primers, enzymes and reagents, buffers, nucleotides, modified nucleotides, reagents for additional assays.

### VIII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

1. A method of assembling a target polynucleotide, comprising:
partitioning a plurality of polynucleotides into a contained reaction volume, wherein:
the plurality of polynucleotides comprise a first polynucleotide and a second polynucleotide, wherein the second polynucleotide is attached to a support,
the first polynucleotide comprises a first subsequence of a target polynucleotide, wherein the first polynucleotide comprises a single-stranded 3′ end sequence,
the second polynucleotide comprises, in the 3′ to 5′ direction:
(i) a single-stranded 3′ end sequence,
(ii) a second subsequence of the target polynucleotide,
(iii) a Type IIS restriction enzyme recognition sequence, and
(iv) a complementary sequence capable of hybridizing to all or a portion of the second subsequence, and
the second polynucleotide is capable of forming a hairpin molecule comprising a 3′ overhang, a stem formed by intramolecular nucleotide base pairing between all or a portion of the second subsequence and the complementary sequence, and a loop, wherein the hairpin molecule is in a configuration that is not cleaved by a Type IIS restriction enzyme;
wherein the first polynucleotide and/or the second polynucleotide optionally further comprise a tag, a barcode, an amplification site, a unique molecular identifier (UMI), or any combination thereof; and
wherein the first and second polynucleotides are connected within the contained reaction volume, thereby assembling the first and second subsequences.

2. The method of claim 1, wherein the first polynucleotide comprises two nucleic acid strands forming a duplex.

3. The method of claim 1 or 2, wherein the first polynucleotide is capable of forming one or more hairpins.

4. The method of any of claims 1-3, wherein the first polynucleotide comprises one or more barcodes and/or one or more tags, e.g., a capture tag sequence.

5. The method of any of claims 1-4, wherein prior to connecting the first and second polynucleotides, the first polynucleotide is not attached to the support.

6. The method of any of claims 1-4, wherein prior to connecting the first and second polynucleotides, the first polynucleotide is attached to the support.

7. The method of claim 6, wherein the first polynucleotide is directly or indirectly attached to the support.

8. The method of claim 6 or 7, wherein the first polynucleotide is covalently or noncovalently attached to the support or a linker, e.g., a cleavable linker.

9. The method of any of claims 6-8, wherein the first polynucleotide is attached to the support via hybridization (e.g., between a capture probe sequence directly or indirectly on the support and a capture tag sequence of the first polynucleotide), the interaction between a binding pair (e.g., biotin/streptavidin binding), a covalent bond, or any combination thereof.

10. The method of any of claims 6-9, wherein the first polynucleotide remains attached to the support during and/or after connecting the first and second polynucleotides.

11. The method of any of claims 6-10, wherein the first polynucleotide is released from the support after the first and second polynucleotides are connected.

12. The method of any of claims 6-9, wherein the first polynucleotide is released from the support before the first and second polynucleotides are connected.

13. The method of any of claims 10-12, wherein the releasing comprises heating the contained reaction volume and/or enzymatic cleavage of the first polynucleotide or a linker, e.g., a cleavable linker.

14. The method of any of claims 1-13, wherein the second polynucleotide comprises one or more barcodes and/or one or more tags, e.g., a capture tag sequence.

15. The method of any of claims 1-14, wherein the second polynucleotide is directly or indirectly attached to the support.

16. The method of any of claims 1-15, wherein the second polynucleotide is covalently or noncovalently attached to the support or a linker, e.g., a cleavable linker.

17. The method of any of claims 1-16, wherein the second polynucleotide is attached to the support via hybridization (e.g., between a capture probe sequence directly or indirectly on the support and a capture tag sequence of the second polynucleotide), the interaction between a binding pair (e.g., biotin/streptavidin binding), a covalent bond, or any combination thereof.

18. The method of any of claims 1-17, wherein prior to connecting the first and second polynucleotides, the second polynucleotide is not released from the support.

19. The method of claim 18, wherein the second polynucleotide remains attached to the support during and/or after connecting the first and second polynucleotides.

20. The method of claim 18 or 19, wherein the second polynucleotide is released from the support after the first and second polynucleotides are connected.

21. The method of any of claims 1-17, wherein prior to connecting the first and second polynucleotides, the second polynucleotide is released from the support.

22. The method of claim 20 or 21, wherein the releasing comprises heating the contained reaction volume and/or enzymatic cleavage of the second polynucleotide or a linker, e.g., a cleavable linker.

23. The method of any of claims 1-22, wherein the first and second polynucleotides are connected in the contained reaction volume when both are not attached to the support.

24. The method of any of claims 1-23, wherein the second polynucleotide forms the hairpin molecule before and/or during connecting the first and second polynucleotides.

25. The method of any of claims 1-24, wherein the 5′ end of the second polynucleotide is blocked from ligation, extension, and/or hybridization.

26. The method of any of claims 1-25, wherein the second polynucleotide further comprises, between the second subsequence and the complementary sequence, a sequence comprising one or more barcodes and/or one or more tags, e.g., a capture tag sequence.

27. The method of claim 26, wherein the sequence comprising one or more barcodes and/or one or more tags is between the Type IIS restriction enzyme recognition sequence and the complementary sequence.

28. The method of any of claims 1-27, wherein the second polynucleotide further comprises a 5′ end sequence that does not hybridize to the single-stranded 3′ end sequence or the second subsequence.

29. The method of claim 28, wherein the 5′ end sequence comprises one or more barcodes and/or one or more tags, e.g., a capture tag sequence.

30. The method of claim 28 or 29, wherein the 5′ end sequence is blocked from ligation, extension, and/or hybridization.

31. The method of any of claims 1-30, wherein the stem comprises one or more bulged bases in either one or both strands of the stem.

32. The method of claim 31, wherein the stem comprises a bulge sequence in the strand comprising the complementary sequence.

33. The method of claim 31 or 32, wherein the bulge sequence is capable of forming one or more internal hairpins.

34. The method of any of claims 31-33, wherein the bulge sequence comprises one or more barcodes and/or one or more tags, e.g., a capture tag sequence.

35. The method of any of claims 31-34, wherein the stem comprises a bulge sequence in the strand comprising the second subsequence.

36. The method of any of claims 1-35, wherein the second subsequence is capable of forming one or more hairpins internal to the hairpin molecule formed by the second polynucleotide.

37. The method of any of claims 1-36, wherein the second polynucleotide further comprises an intervening sequence between the second subsequence and the Type IIS restriction enzyme recognition sequence.

38. The method of claim 37, wherein the intervening sequence is capable of being cleaved from the second subsequence by the Type IIS restriction enzyme when the second polynucleotide forms a duplex with a complementary strand.

39. The method of any of claims 1-36, wherein there is no intervening sequence between the second subsequence and the Type IIS restriction enzyme recognition sequence.

40. The method of any of claims 1-39, wherein the 3′ end of the 3′ overhang is not blocked from ligation, extension, and/or hybridization.

41. The method of any of claims 1-40, wherein the 3′ overhang is between about 1 and about 100 nucleotides in length.

42. The method of any of claims 1-41, wherein the 3′ overhang is between about 2 and about 20 nucleotides in length.

43. The method of any of claims 1-42, wherein the 3′ overhang is between about 2 and about 15 nucleotides in length, e.g., 2, 3, 4, 5, 6, 7, 8, 9, or 10 nucleotides in length.

44. The method of any of claims 1-43, wherein the contained reaction volume is an emulsion droplet.

45. The method of any of claims 1-44, wherein the contained reaction volume comprises one or more Type IIS restriction enzymes.

46. The method of any of claims 1-45, wherein the contained reaction volume comprises one or more polymerases.

47. The method of any of claims 1-46, wherein the contained reaction volume comprises one or more ligases.

48. The method of any of claims 1-47, wherein the contained reaction volume comprises one or more nucleases other than a Type IIS restriction enzyme, e.g., one or more exonucleases and/or one or more endonucleases.

49. The method of any of claims 1-48, wherein the second polynucleotide forms the hairpin molecule, and all or a portion of the 3′ overhang hybridizes to all or a portion of the single-stranded 3′ end sequence of the first subsequence to form a hybridization complex.

50. The method of claim 49, wherein the hybridization complex comprises (i) a nick or gap between the 3′ end of the first polynucleotide and the 5′ end of the second polynucleotide, and (ii) a nick or gap between the 5′ end of the first polynucleotide and the 3′ end of the second polynucleotide.

51. The method of claim 49 or 50, wherein a polymerase is capable of extending the 3′ end sequence of the first subsequence in the hybridization complex using the second polynucleotide as template.

52. The method of claim 49 or 50, wherein a polymerase is incapable of extending the 3′ end sequence of the first subsequence in the hybridization complex using the second polynucleotide as template, e.g., when the hybridization complex comprises two nicks, one on each strand, that are between about 1 and about 10 nucleotides apart, e.g., between about 1 and about 6 nucleotides apart.

53. The method of claim 52, wherein the nick or gap between the 5′ end of the first polynucleotide and the 3′ end of the second polynucleotide is filled, e.g., by ligation of the nick, or by hybridization of a filler sequence to fill in the gap followed by ligation of the filler sequence.

54. The method of claim 52 or 53, wherein the nick between the 5′ end of the first polynucleotide and the 3′ end of the second polynucleotide is ligated by a ligase, whereas the nick between the 3′ end of the first polynucleotide and the 5′ end of the second polynucleotide is not ligated by the ligase, e.g., wherein the 5′ end of the second polynucleotide is blocked from ligation, e.g., wherein the 5′ end nucleotide of the second polynucleotide is dephosphorylated.

55. The method of any of claims 51-54, wherein a double-stranded polynucleotide comprising the first subsequence, the second subsequence, the Type IIS restriction enzyme recognition sequence, and optionally the complementary sequence, is generated by a polymerase that extends the 3′ end sequence of the first subsequence using the second polynucleotide as template.

56. The method of claim 55, wherein a Type IIS restriction enzyme recognizes the Type IIS restriction enzyme recognition sequence and cleaves the double-stranded polynucleotide, thereby generating a cleaved double-stranded polynucleotide comprising the first subsequence connected to the second subsequence.

57. The method of claim 56, wherein the cleaved double-stranded polynucleotide comprises a single-stranded 3′ end sequence.

58. The method of claim 57, wherein the single-stranded 3′ end sequence of the cleaved double-stranded polynucleotide is between about 2 and about 10 nucleotides in length.

59. The method of any of claims 1-58, wherein the plurality of polynucleotides further comprise a third polynucleotide.

60. The method of claim 59, wherein the third polynucleotides is attached to the support and comprises, in the 3′ to 5′ direction:
(i) a single-stranded 3′ end sequence,
(ii) a third subsequence of the target polynucleotide,
(iii) a Type IIS restriction enzyme recognition sequence, and
(iv) a complementary sequence capable of hybridizing to all or a portion of the third subsequence,
wherein the third polynucleotide is capable of forming a hairpin molecule comprising a 3′ overhang, a stem formed by intramolecular nucleotide base pairing between all or a portion of the third subsequence and the complementary sequence, and a loop, wherein the hairpin molecule is in a configuration that is not cleaved by a Type IIS restriction enzyme, and
wherein the first, second, and third polynucleotides are connected sequentially within the contained reaction volume, thereby assembling the first, second, and third subsequences.

61. The method of any of claims 1-60, wherein the support comprises a particle, a bead, a solid substrate, a plate, a well, an array, a membrane, or a combination thereof.

62. The method of any of claims 1-61, wherein the target polynucleotide is at least about 100, about 250, about 500, about 1,000, about 2,500, about 5,000, about 10,000, about 25,000, or about 50,000 nucleotides in length.

63. The method of any of claims 1-62, wherein the plurality of polynucleotides comprise 3, 4, 5, 6, 7, 8, 9, 10 or more polynucleotides each comprising a subsequence of the target polynucleotide.

64. The method of any of claims 1-63, wherein the target polynucleotide is a DNA molecule, and the target polynucleotide optionally comprises a gene or fragment thereof, a gene cluster, a mitochondrial DNA or fragment thereof, a chromosome or fragment thereof, or a genome.

65. The method of any of claims 1-64, wherein the first polynucleotide and/or the second polynucleotide further comprise a capture tag sequence, an amplification site, and a UMI, wherein the UMI sequence is complementary to the capture tag sequence and/or the amplification site.

66. A method of assembling a plurality of target polynucleotides, comprising:
(a) for each target polynucleotide, partitioning a plurality of polynucleotides into a contained reaction volume, wherein:
the plurality of polynucleotides comprise a first polynucleotide and a second polynucleotide, wherein the second polynucleotide is attached to a support,
the first polynucleotide comprises a first subsequence of the target polynucleotide, wherein the first polynucleotide comprises a single-stranded 3′ end sequence,
the second polynucleotide comprises, in the 3′ to 5′ direction:
(i) a single-stranded 3′ end sequence,
(ii) a second subsequence of the target polynucleotide,
(iii) a Type IIS restriction enzyme recognition sequence, and
(iv) a complementary sequence capable of hybridizing to all or a portion of the second subsequence, and
the second polynucleotide is capable of forming a hairpin molecule comprising a 3′ overhang, a stem formed by intramolecular nucleotide base pairing between all or a portion of the second subsequence and the complementary sequence, and a loop, wherein the hairpin molecule is in a configuration that is not cleaved by a Type IIS restriction enzyme; and
(b) within each contained reaction volume, connecting the first and second polynucleotides, thereby assembling the first and second subsequences,
wherein the assembly of subsequences of each target polynucleotide is carried out in parallel.

67. The method of claim 66, further comprising designing and/or obtaining the plurality of polynucleotides for each target polynucleotide.

68. The method of claim 66 or 67, wherein the subsequences in the plurality of polynucleotides for each target polynucleotide are between about 20 and about 200 nucleotides in length.

69. The method of any of claims 66-68, wherein the plurality of polynucleotides for each target polynucleotide are synthesized, and the synthesis comprises base-by-base synthesis.

70. The method of any of claims 66-69, wherein the partitioning comprises enriching polynucleotides comprising subsequences of a given target polynucleotide, but not polynucleotides comprising subsequences of other target polynucleotides, in the contained reaction volume.

71. The method of any of claims 66-70, wherein the partitioning comprises capturing all or a subset of the plurality of polynucleotides for each target polynucleotide on a bead that is specific for the target polynucleotide.

72. The method of claim 71, wherein the bead comprises a capture probe that specifically binds to a capture tag that is unique for the target polynucleotide, wherein the capture tag is common in all or a subset of the plurality of polynucleotides comprising subsequences of the target polynucleotide.

73. The method of claim 71 or 72, wherein the partitioning comprises encapsulating the bead in an emulsion droplet, thereby generating a plurality of emulsion droplets for parallel assembly of the plurality of target polynucleotides.

74. The method of claim 73, further comprising releasing all or a subset of the polynucleotides captured on the beads into the emulsion droplets.

75. The method of claim 73 or 74, wherein the parallel assembly of the plurality of target polynucleotides is carried out in each emulsion droplet by one or more concerted reaction cycles.

76. The method of claim 75, wherein the one or more concerted reaction cycles comprise an isothermal reaction.

77. The method of claim 75 or 76, wherein the one or more concerted reaction cycles comprise sequential reactions of hybridization, ligation by a ligase, primer extension by a polymerase, and cleavage by a Type IIS restriction enzyme.

78. The method of any of claims 66-77, wherein the assembly of all or a subset of the plurality of target polynucleotides is unidirectional.

79. The method of any of claims 66-78, wherein the assembly of all or a subset of the plurality of target polynucleotides is bidirectional.

80. A method of assembling a target polynucleotide, comprising:
(a) partitioning a plurality of polynucleotides into an emulsion droplet, wherein:
the plurality of polynucleotides comprise: (i) a first polynucleotide optionally attached to a bead, and (ii) a second polynucleotide attached to the bead,
the first polynucleotide comprises a first subsequence of a target polynucleotide, wherein the first polynucleotide comprises a single-stranded 3′ end sequence,
the second polynucleotide comprises, in the 3′ to 5′ direction:
(i) a single-stranded 3′ end sequence capable of hybridizing to the single-stranded 3′ end sequence of the first polynucleotide,
(ii) a second subsequence of the target polynucleotide,
(iii) a Type IIS restriction enzyme recognition sequence, and
(iv) a complementary sequence capable of hybridizing to all or a portion of the second subsequence, and
the second polynucleotide further comprises a tag sequence and/or a barcode sequence 5′ to the Type IIS restriction enzyme recognition sequence;
(b) in the emulsion droplet, releasing the second polynucleotide from the bead, wherein the second polynucleotide forms a hairpin molecule comprising a 3′ overhang, a stem formed by intramolecular nucleotide base pairing between all or a portion of the second subsequence and the complementary sequence, and a loop, wherein the hairpin molecule is in a configuration that is not cleaved by a Type IIS restriction enzyme;
(c) allowing the 3′ overhang of the hairpin molecule to hybridize to the single-stranded 3′ end sequence of the first polynucleotide, wherein the 5′ end of the hairpin molecule is optionally blocked from ligation to the 3′ end of the first polynucleotide after hybridization;
(d) optionally ligating the 3′ end of the hairpin molecule to the 5′ end of the first polynucleotide;
(e) extending the 3′ end sequence of the first polynucleotide using the second polynucleotide as template, thereby generating a double-stranded polynucleotide comprising the first subsequence, the second subsequence, the Type IIS restriction enzyme recognition sequence, and optionally the complementary sequence, the tag sequence, and/or the barcode sequence; and
(f) cleaving the double-stranded polynucleotide using a Type IIS restriction enzyme, thereby generating a cleaved double-stranded polynucleotide comprising the first subsequence and the second subsequence, wherein the cleaved double-stranded polynucleotide comprises a single-stranded 3′ end sequence, and optionally wherein the single-stranded 3′ end sequence is between about 2 and about 10 nucleotides in length,
thereby assembling the first and second subsequences.

81. The method of claim 80, wherein the first polynucleotide is attached to the bead prior to the partitioning step.

82. The method of claim 80, wherein the partitioning step comprises attaching the first polynucleotide and the second polynucleotide to the bead, and the releasing step optionally comprises releasing the first polynucleotide from the bead.

83. The method of any of claims 80-82, wherein the first polynucleotide and/or the second polynucleotide are directly or indirectly attached to the bead.

84. The method of any of claims 80-83, wherein the first polynucleotide and/or the second polynucleotide are covalently or noncovalently attached to the bead or a linker, e.g., a cleavable linker.

85. The method of any of claims 80-84, wherein the first polynucleotide and/or the second polynucleotide are attached to the bead via hybridization (e.g., between a capture probe sequence directly or indirectly on the bead and a capture tag sequence of the first polynucleotide and/or the second polynucleotide), the interaction between a binding pair (e.g., biotin/streptavidin binding), a covalent bond, or any combination thereof.

86. The method of claim 80, wherein the first polynucleotide is not attached to the bead prior to, during, or after the partitioning step.

87. The method of claim 86, wherein the first polynucleotide is provided in a reaction volume that is partitioned to form the emulsion droplet.

88. The method of claim 87, wherein the reaction volume further comprises a ligase, a polymerase, a Type IIS restriction enzyme, and/or a nuclease other than a Type IIS restriction enzyme.

89. The method of any of claims 80-88, wherein the first polynucleotide comprises a hairpin.

90. The method of claim 89, wherein the first polynucleotide comprises a stem comprising all or a portion of the first subsequence and a loop comprising a tag sequence and/or a barcode sequence.

91. The method of any of claims 80-90, wherein:
in the partitioning step, the plurality of polynucleotides further comprise (iii) a third polynucleotide attached to the bead,
the third polynucleotide comprises, in the 3′ to 5′ direction:
(i) a single-stranded 3′ end sequence capable of hybridizing to the single-stranded 3′ end sequence of the cleaved double-stranded polynucleotide,
(ii) a third subsequence of the target polynucleotide,
(iii) a Type IIS restriction enzyme recognition sequence, and
(iv) a complementary sequence capable of hybridizing to all or a portion of the third subsequence, and
the third polynucleotide further comprises a tag sequence and/or a barcode sequence 5′ to the Type IIS restriction enzyme recognition sequence.

92. The method of claim 91, wherein:
the releasing step further comprises releasing the third polynucleotide from the bead, wherein the third polynucleotide forms a hairpin molecule comprising a 3′ overhang, a stem formed by intramolecular nucleotide base pairing between all or a portion of the third subsequence and the complementary sequence, and a loop, wherein the hairpin molecule is in a configuration that is not cleaved by a Type IIS restriction enzyme.

93. The method of claim 92, further comprising:
(g) hybridizing the 3′ overhang of the hairpin molecule formed by the third polynucleotide to the single-stranded 3′ end sequence of the cleaved double-stranded polynucleotide, wherein the 5′ end of the hairpin molecule formed by the third polynucleotide is blocked from ligation to the 3′ end of the first polynucleotide after hybridization.

94. The method of claim 93, further comprising:
(h) ligating the 3′ end of the hairpin molecule formed by the third polynucleotide to the 5′ end of the cleaved double-stranded polynucleotide.

95. The method of claim 94, further comprising:
(i) extending the 3′ end sequence of the cleaved double-stranded polynucleotide using the third polynucleotide as template, thereby generating a double-stranded polynucleotide comprising the first subsequence, the second subsequence, the third subsequence, the Type IIS restriction enzyme recognition sequence of the third polynucleotide, and optionally the complementary sequence, the tag sequence, and/or the barcode sequence of the third polynucleotide.

96. The method of claim 95, further comprising:
(j) cleaving the double-stranded polynucleotide using a Type IIS restriction enzyme, thereby generating a cleaved double-stranded polynucleotide comprising the first subsequence, the second subsequence, and the third subsequence, wherein the cleaved double-stranded polynucleotide comprises a single-stranded 3′ end sequence, and optionally wherein the single-stranded 3′ end sequence is between about 2 and about 10 nucleotides in length, thereby assembling the first, second, and third subsequences.

97. The method of any of claims 80-96, wherein:
in the partitioning step, the plurality of polynucleotides further comprise an n^{th} polynucleotide attached to the bead, wherein n is an integer of 4 or greater,
the n^{th} polynucleotide comprises, in the 3′ to 5′ direction:
(i) a single-stranded 3′ end sequence capable of hybridizing to the single-stranded 3′ end sequence of a cleaved double-stranded polynucleotide comprising the first, second, . . . , and the (n−1)^{th} subsequences of the target polynucleotide,
(ii) an n^{th} subsequence of the target polynucleotide,
(iii) a Type IIS restriction enzyme recognition sequence, and
(iv) a complementary sequence capable of hybridizing to all or a portion of the n^{th} subsequence, and
the n^{th} polynucleotide further comprises a tag sequence and/or a barcode sequence 5′ to the Type IIS restriction enzyme recognition sequence.

98. The method of claim 97, wherein:
the releasing step further comprises releasing the n^{th} polynucleotide from the bead, wherein the n^{th} polynucleotide forms a hairpin molecule comprising a 3′ overhang, a stem formed by intramolecular nucleotide base pairing between all or a portion of the n^{th} subsequence and the complementary sequence, and a loop, wherein the hairpin molecule is in a configuration that is not cleaved by a Type IIS restriction enzyme.

99. The method of claim 98, further comprising repeating a concerted reaction cycle comprising sequential reactions of hybridization, ligation by a ligase, primer extension by a polymerase, and cleavage by a Type IIS restriction enzyme, thereby assembling the first, second, . . . , and the (n−1)^{th} subsequences.

100. A method of assembling a target polynucleotide, comprising:
(a) partitioning a plurality of polynucleotides into an emulsion droplet, wherein:
the plurality of polynucleotides comprise: (i) a first polynucleotide optionally attached to a bead, (ii) a second polynucleotide attached to the bead, and (iii) a third polynucleotide attached to the bead,
the first polynucleotide comprises a first subsequence of a target polynucleotide and is double-stranded, comprising a single-stranded 3′ end sequence in the top strand and a single-stranded 3′ end sequence in the bottom strand,
the second polynucleotide comprises, in the 3′ to 5′ direction:
(i) a single-stranded 3′ end sequence capable of hybridizing to the top strand single-stranded 3′ end sequence of the first polynucleotide,
(ii) a second subsequence of the target polynucleotide,
(iii) a Type IIS restriction enzyme recognition sequence, and
(iv) a complementary sequence capable of hybridizing to all or a portion of the second subsequence,
the second polynucleotide optionally further comprises a tag sequence and/or a barcode sequence 5′ to the Type IIS restriction enzyme recognition sequence,
the third polynucleotide comprises, in the 3′ to 5′ direction:
(i) a single-stranded 3′ end sequence capable of hybridizing to the bottom strand single-stranded 3′ end sequence of the first polynucleotide,
(ii) a third subsequence of the target polynucleotide,
(iii) a Type IIS restriction enzyme recognition sequence, and
(iv) a complementary sequence capable of hybridizing to all or a portion of the third subsequence,
the third polynucleotide optionally further comprises a tag sequence and/or a barcode sequence 5′ to the Type IIS restriction enzyme recognition sequence;
(b) in the emulsion droplet, releasing the second and third polynucleotides, and optionally the first polynucleotide, from the bead, wherein:
the second polynucleotide forms a hairpin molecule comprising a 3′ overhang, a stem formed by intramolecular nucleotide base pairing between all or a portion of the second subsequence and the complementary sequence, and a loop, wherein the hairpin molecule is in a configuration that is not cleaved by a Type IIS restriction enzyme, and
the third polynucleotide forms a hairpin molecule comprising a 3′ overhang, a stem formed by intramolecular nucleotide base pairing between all or a portion of the third subsequence and the complementary sequence, and a loop, wherein the hairpin molecule is in a configuration that is not cleaved by a Type IIS restriction enzyme;
(c) allowing the 3′ overhangs of the hairpin molecules formed by the second and third polynucleotides to hybridize to the top strand single-stranded 3′ end sequence and the bottom strand single-stranded 3′ end sequence, respectively, of the first polynucleotide, wherein the 5′ ends of the hairpin molecules are blocked from ligation to the 3′ ends of the first polynucleotide after hybridization;
(d) ligating the 3′ ends of the hairpin molecules to the 5′ ends of the first polynucleotide;
(e) extending the 3′ end sequences of the first polynucleotide using the second and third polynucleotides as template, thereby generating a double-stranded polynucleotide comprising the first subsequence flanked by the second subsequence on one side and the third subsequence on the other side, the Type IIS restriction enzyme recognition sequences, and optionally the complementary sequences, the tag sequence(s), and/or the barcode sequence(s); and
(f) cleaving the double-stranded polynucleotide using a Type IIS restriction enzyme, thereby generating a cleaved double-stranded polynucleotide comprising the first subsequence flanked by the second subsequence on one side and the third subsequence on the other side, wherein the cleaved double-stranded polynucleotide comprises a single-stranded 3′ end sequence in the top strand and a single-stranded 3′ end sequence in the bottom strand, and optionally wherein the single-stranded 3′ end sequences are between about 2 and about 10 nucleotides in length,
thereby assembling the first, second, and third subsequences.

101. The method of claim 100, wherein:
in the partitioning step, the plurality of polynucleotides further comprise a fourth polynucleotide attached to the bead and optionally a fifth polynucleotide attached to the bead,
the fourth polynucleotide comprises, in the 3′ to 5′ direction:
(i) a single-stranded 3′ end sequence capable of hybridizing to the top strand single-stranded 3′ end sequence of the cleaved double-stranded polynucleotide,
(ii) a fourth subsequence of the target polynucleotide,
(iii) a Type IIS restriction enzyme recognition sequence, and
(iv) a complementary sequence capable of hybridizing to all or a portion of the fourth subsequence, and
the fourth polynucleotide optionally further comprises a tag sequence and/or a barcode sequence 5′ to the Type IIS restriction enzyme recognition sequence,
the optional fifth polynucleotide comprises, in the 3′ to 5′ direction:
(i) a single-stranded 3′ end sequence capable of hybridizing to the bottom strand single-stranded 3′ end sequence of the cleaved double-stranded polynucleotide,
(ii) a fifth subsequence of the target polynucleotide,
(iii) a Type IIS restriction enzyme recognition sequence, and
(iv) a complementary sequence capable of hybridizing to all or a portion of the fifth subsequence, and
the fifth polynucleotide optionally further comprises a tag sequence and/or a barcode sequence 5′ to the Type IIS restriction enzyme recognition sequence.

102. The method of claim 101, wherein:
the releasing step further comprises releasing the fourth and fifth polynucleotides from the bead, wherein the fourth polynucleotide forms a hairpin molecule comprising a 3′ overhang, a stem formed by intramolecular nucleotide base pairing between all or a portion of the fourth subsequence and the complementary sequence, and a loop, wherein the hairpin molecule is in a configuration that is not cleaved by a Type IIS restriction enzyme, and
the fifth polynucleotide forms a hairpin molecule comprising a 3′ overhang, a stem formed by intramolecular nucleotide base pairing between all or a portion of the fifth subsequence and the complementary sequence, and a loop comprising the Type IIS restriction enzyme recognition sequence in a configuration that is not cleaved by a Type IIS restriction enzyme.

103. The method of claim 102, further comprising:
(g) hybridizing the 3′ overhangs of the hairpin molecules formed by the fourth and fifth polynucleotides to the top strand single-stranded 3′ end sequence and the bottom strand single-stranded 3′ end sequence, respectively, of the cleaved double-stranded polynucleotide, wherein the 5′ ends of the hairpin molecules are blocked from ligation to the 3′ ends of the cleaved double-stranded polynucleotide after hybridization.

104. The method of claim 103, further comprising:
(h) ligating the 3′ ends of the hairpin molecules formed by the fourth and fifth polynucleotides to the 5′ ends of the cleaved double-stranded polynucleotide.

105. The method of claim 104, further comprising:
(i) extending the 3′ end sequences of the cleaved double-stranded polynucleotide using the fourth and fifth polynucleotides as template, thereby generating a double-stranded polynucleotide comprising: the first subsequence flanked by the second subsequence on one side and the third subsequence on the other side, which are in turn flanked by the fourth subsequence and the fifth subsequence, respectively; the Type IIS restriction enzyme recognition sequences of the fourth and fifth polynucleotides; and optionally the complementary sequences, the tag sequence(s), and/or the barcode sequence(s) of the fourth and fifth polynucleotides.

106. The method of claim 105, further comprising:
(j) cleaving the double-stranded polynucleotide using a Type IIS restriction enzyme, thereby generating a cleaved double-stranded polynucleotide comprising the first subsequence flanked by the second subsequence on one side and the third subsequence on the other side, which are in turn flanked by the fourth subsequence and the fifth subsequence, respectively, wherein the cleaved double-stranded polynucleotide comprises a single-stranded 3′ end sequence in the top strand and a single-stranded 3′ end sequence in the bottom strand, and optionally wherein the single-stranded 3′ end sequences are between about 2 and about 10 nucleotides in length,
thereby assembling the first, second, third, fourth, and fifth subsequences.

107. A method of assembling a target polynucleotide, comprising:
(a) partitioning a plurality of polynucleotides into an emulsion droplet, wherein:
the plurality of polynucleotides comprise: (i) a first polynucleotide optionally attached to a bead, and (ii) a second polynucleotide attached to the bead,
the first polynucleotide comprises a first subsequence of a target polynucleotide, wherein the first polynucleotide comprises a single-stranded 3′ end sequence,
the second polynucleotide comprises, in the 3′ to 5′ direction:
(i) a single-stranded 3′ end sequence capable of hybridizing to the single-stranded 3′ end sequence of the first polynucleotide,
(ii) a second subsequence of the target polynucleotide,
(iii) a Type IIS restriction enzyme recognition sequence, and
(iv) a complementary sequence capable of hybridizing to all or a portion of the second subsequence, and
the second polynucleotide further comprises a tag sequence and/or a barcode sequence 5′ to the Type IIS restriction enzyme recognition sequence;
(b) in the emulsion droplet, releasing the second polynucleotide from the bead, wherein the second polynucleotide forms a hairpin molecule comprising a 3′ overhang, a stem formed by intramolecular nucleotide base pairing between all or a portion of the second subsequence and the complementary sequence, and a loop, wherein the hairpin molecule is in a configuration that is not cleaved by a Type IIS restriction enzyme;
(c) allowing the 3′ overhang of the hairpin molecule to hybridize to the single-stranded 3′ end sequence of the first polynucleotide to form a hybridization complex, wherein:
the 5′ end of the hairpin molecule is blocked from ligation to the 3′ end of the first polynucleotide after hybridization, and
the hybridization complex comprises (i) a nick or gap between the 3′ end of the first polynucleotide and the 5′ end of the second polynucleotide, and (ii) a nick or gap between the 5′ end of the first polynucleotide and the 3′ end of the second polynucleotide,
optionally wherein the nicks and gaps are more than about 6-10 nucleotides apart;
(d) extending the 3′ end sequence of the first polynucleotide using the second polynucleotide as template, thereby generating a double-stranded polynucleotide comprising the first subsequence, the second subsequence, the Type IIS restriction enzyme recognition sequence, and optionally the complementary sequence, the tag sequence, and/or the barcode sequence; and
(e) cleaving the double-stranded polynucleotide using a Type IIS restriction enzyme, thereby generating a cleaved double-stranded polynucleotide comprising the first subsequence and the second subsequence, wherein the cleaved double-stranded polynucleotide comprises a single-stranded 3′ end sequence, and optionally wherein the single-stranded 3′ end sequence is between about 2 and about 10 nucleotides in length,
thereby assembling the first and second subsequences.

108. The method of claim 107, wherein the emulsion droplet comprises a ligase, a polymerase, and a Type IIS restriction enzyme, and optionally a nuclease other than a Type IIS restriction enzyme.

109. A method, comprising contacting a pool of polynucleotides with a library of beads, wherein:
the pool of polynucleotides comprises polynucleotide sets P11, . . . , and P1j₁; . . . ; Pk1, . . . , and Pkjₖ; . . . ; and Pi1, . . . , and Pijᵢ, wherein i, j₁, . . . , jₖ, . . . , jᵢ, and k are integers, i, j₁, . . . , jₖ, . . . , and jᵢ are independently 2 or greater, and 1≤k≤i,
Pk1, . . . , and Pkjₖ comprise subsequences Sk1, . . . , and Skjₖ, respectively, which form target sequence S′k,
at least one of Pk1, . . . , and Pkjₖ comprises, in the 3′ to 5′ direction:
(i) a single-stranded 3′ end sequence,
(ii) the subsequence of target sequence S′k,
(iii) a Type IIS restriction enzyme recognition sequence, and
(iv) a complementary sequence capable of hybridizing to all or a portion of the subsequence of target sequence S′k,
the at least one of Pk1, . . . , and Pkjₖ further comprises a tag Tk in all or a subset of Pk1, . . . , and Pkjₖ, and
the at least one of Pk1, . . . , and Pkjₖ is capable of forming a hairpin molecule comprising a 3′ overhang, a stem formed by intramolecular nucleotide base pairing between all or a portion of the subsequence of target sequence S′k and the complementary sequence, and a loop, wherein the hairpin molecule is in a configuration that is not cleaved by a Type IIS restriction enzyme;
beads B1, . . . , Bk, . . . , and Bi in the library comprise capture moieties C1, . . . , Ck, . . . , and Ci, respectively, that specifically binds to tags T1, . . . , Tk, . . . , and Ti, respectively, thereby specifically capturing the at least one of Pk1, . . . , and Pkjₖ on one of the beads in the library.

110. The method of claim 109, further comprising placing all or a subset of the beads in emulsion droplets, one bead per emulsion droplet.

111. The method of claim 110, further comprising releasing all or a subset of the polynucleotides captured on each of all or a subset of the beads in the emulsion droplets.

112. The method of claim 111, further comprising within each emulsion droplet, connecting two or more of Pk1, . . . , and Pkjₖ, thereby assembling two or more of subsequences Sk1, . . . , and Skjₖ, in the emulsion droplet.

113. The method of claim 112, wherein Pk1, . . . , and Pkjₖ are assembled in the emulsion droplet by one or more concerted reaction cycles.

114. The method of claim 113, wherein the one or more concerted reaction cycles comprise an isothermal reaction.

115. The method of claim 113 or 114, wherein the one or more concerted reaction cycles comprise sequential reactions of hybridization, ligation by a ligase, primer extension by a polymerase, and cleavage by a Type IIS restriction enzyme.

116. The method of any of claims 113-115, wherein the one or more concerted reaction cycles comprise sequential assembly of all or a subset of Pk1, . . . , and Pkjₖ in a predetermined order.

117. The method of any of claims 112-116, wherein subsequence sets S11, . . . , and S1j₁; . . . ; Sk1, . . . , and Skjₖ; . . . ; and Si1, . . . , and Sijᵢ comprise one or more common subsequences among two or more of the subsequence sets.

118. The method of any of claims 112-117, wherein polynucleotide sets P11, . . . , and P1j₁; . . . ; Pk1, . . . , and Pkjₖ; . . . ; and Pi1, . . . , and Pijᵢ comprise one or more common polynucleotides among two or more of the polynucleotide sets.

119. The method of any of claims 112-116, wherein subsequence sets S11, . . . , and S1j₁; . . . ; Sk1, . . . , and Skjₖ; . . . ; and Si1, . . . , and Sijᵢ do not contain a common subsequence.

120. The method of any of claims 112-119, wherein Pk1, . . . , and Pkjₖ are assembled to form target sequence S′k or a portion thereof.

121. The method of any of claims 112-120, wherein polynucleotide sets P11, . . . , and P1j₁; . . . ; Pk1, . . . , and Pkjₖ; . . . ; and Pi1, . . . , and Pijᵢ are assembled to form target sequences S′1, . . . , S′k, . . . , and S′i or a portion thereof, respectively, in parallel.

122. The method of any of claims 112-121, further comprising breaking the emulsion droplets and pooling all or a subset of the assembled target sequences or portions thereof.

123. The method of any of claims 112-122, wherein all or a subset of the assembled target sequences or portions thereof are subjected to further assembly.

124. The method of claim 123, wherein the further assembly comprises higher order assembly of all or a subset of the assembled target sequences or portions thereof.

125. The method of claim 123 or 124, wherein the further assembly comprises polymerase cycling assembly (PCA), sequence- and ligation-independent cloning (SLIC), Golden Gate assembly, Gibson assembly, in vivo assembly, or any combination thereof.

126. The method of any of claims 1-125, wherein the target sequence comprises a sequence difficult to synthesize, difficult to amplify, and/or difficult to sequence verify.

127. The method of any of claims 1-126, wherein the target sequence comprises a sequence difficult to synthesize base-by-base.

128. The method of any of claims 1-127, wherein the target sequence comprises a homopolymer sequence, e.g., Aₙ; a homocopolymer sequence, e.g., [AT]ₙ; a sequence comprising direct repeats; an AT-rich sequence; a GC-rich sequence, or any combination thereof.


SEQUENCE LISTING (108 sequences)

SEQ ID NO: 1 - DNA, 22 bp, Artificial Sequence
  Synthetic construct
  misc_feature 7, 8, 9, 10, 11, 12, 13, 14, 15, 16, 17, 18, 19, 20, 21,22: n = A,T,C or G
ctgaagnnnnnnnnnnnnnnnn

SEQ ID NO: 2 - DNA, 20 bp, Artificial Sequence
  Synthetic construct
  misc_feature 1, 2, 3, 4, 5, 6, 7, 8, 9, 10, 11, 12, 13, 14: n = A,T,C or G
nnnnnnnnnnnnnncttcag

SEQ ID NO: 3 - DNA, 9 bp, Artificial Sequence
  Synthetic construct
  misc_feature 6, 7, 8, 9: n = A,T,C or G
ggatcnnnn

SEQ ID NO: 4 - DNA, 10 bp, Artificial Sequence
  Synthetic construct
  misc_feature 1, 2, 3, 4, 5: n = A,T,C or G
nnnnngatcc

SEQ ID NO: 5 - DNA, 33 bp, Artificial Sequence
  Synthetic construct
  misc_feature 1, 2, 3, 4, 5, 6, 7, 8, 9, 10, 13, 14, 15, 16, 22, 23,24, 25, 26, 27, 28, 29, 30, 31, 32, 33: n = A,T,C or G
nnnnnnnnnnacnnnngtaycnnnnnnnnnnnn

SEQ ID NO: 6 - DNA, 33 bp, Artificial Sequence
  Synthetic construct
  misc_feature 1, 2, 3, 4, 5, 6, 7, 13, 14, 15, 16, 19, 20, 21, 22, 23,24, 25, 26, 27, 28, 29, 30, 31, 32, 33: n = A,T,C or G
nnnnnnngrtacnnnngtnnnnnnnnnnnnnnn

SEQ ID NO: 7 - DNA, 8 bp, Artificial Sequence
  Synthetic construct
  misc_feature 7, 8: n = A,T,C or G
gaagacnn

SEQ ID NO: 8 - DNA, 12 bp, Artificial Sequence
  Synthetic construct
  misc_feature 1, 2, 3, 4, 5, 6: n = A,T,C or G
nnnnnngtcttc

SEQ ID NO: 9 - DNA, 8 bp, Artificial Sequence
  Synthetic construct
  misc_feature 7, 8: n = A,T,C or G
gaagacnn

SEQ ID NO: 10 - DNA, 12 bp, Artificial Sequence
  Synthetic construct
  misc_feature 1, 2, 3, 4, 5, 6: n = A,T,C or G
nnnnnngtcttc

SEQ ID NO: 11 - DNA, 13 bp, Artificial Sequence
  Synthetic construct
  misc_feature 6, 7, 8, 9, 10, 11, 12, 13: n = A,T,C or G
gcagcnnnnnnnn

SEQ ID NO: 12 - DNA, 17 bp, Artificial Sequence
  Synthetic construct
  misc_feature 1, 2, 3, 4, 5, 6, 7, 8, 9, 10, 11, 12: n = A,T,C or G
nnnnnnnnnnnngctgc

SEQ ID NO: 13 - DNA, 9 bp, Artificial Sequence
  Synthetic construct
  misc_feature 6, 7, 8, 9: n = A,T,C or G
ccatcnnnn

SEQ ID NO: 14 - DNA, 10 bp, Artificial Sequence
  Synthetic construct
  misc_feature 1, 2, 3, 4, 5: n = A,T,C or G
nnnnngatgg

SEQ ID NO: 15 - DNA, 17 bp, Artificial Sequence
  Synthetic construct
  misc_feature 6, 7, 8, 9, 10, 11, 12, 13, 14, 15, 16, 17: n = A,T,C or G
acggcnnnnnnnnnnnn

SEQ ID NO: 16 - DNA, 19 bp, Artificial Sequence
  Synthetic construct
  misc_feature 1, 2, 3, 4, 5, 6, 7, 8, 9, 10, 11, 12, 13, 14: n = A,T,C or G
nnnnnnnnnnnnnngccgt

SEQ ID NO: 17 - DNA, 34 bp, Artificial Sequence
  Synthetic construct
  misc_feature 1, 2, 3, 4, 5, 6, 7, 8, 9, 10, 14, 15, 16, 17, 18, 19,23, 24, 25, 26, 27, 28, 29, 30, 31, 32, 33, 34: n = A,T,C or G
nnnnnnnnnncgannnnnntgcnnnnnnnnnnnn

SEQ ID NO: 18 - DNA, 34 bp, Artificial Sequence
  Synthetic construct
  misc_feature 1, 2, 3, 4, 5, 6, 7, 8, 9, 10, 14, 15, 16, 17, 18, 19,23, 24, 25, 26, 27, 28, 29, 30, 31, 32, 33, 34: n = A,T,C or G
nnnnnnnnnngcannnnnntcgnnnnnnnnnnnn

SEQ ID NO: 19 - DNA, 12 bp, Artificial Sequence
  Synthetic construct
  misc_feature 7, 8, 9, 10, 11, 12: n = A,T,C or G
gtatccnnnnnn

SEQ ID NO: 20 - DNA, 11 bp, Artificial Sequence
  Synthetic construct
  misc_feature 1, 2, 3, 4, 5: n = A,T,C or G
nnnnnggatac

SEQ ID NO: 21 - DNA, 6 bp, Artificial Sequence
  Synthetic construct
  misc_feature 6: n = A,T,C or G
gtctcn

SEQ ID NO: 22 - DNA, 10 bp, Artificial Sequence
  Synthetic construct
  misc_feature 1, 2, 3, 4, 5: n = A,T,C or G
nnnnngagac

SEQ ID NO: 23 - DNA, 11 bp, Artificial Sequence
  Synthetic construct
  misc_feature 7, 8, 9, 10, 11: n = A,T,C or G
actgggnnnnn

SEQ ID NO: 24 - DNA, 10 bp, Artificial Sequence
  Synthetic construct
  misc_feature 1, 2, 3, 4: n = A,T,C or G
nnnncccagt

SEQ ID NO: 25 - DNA, 10 bp, Artificial Sequence
  Synthetic construct
  misc_feature 7, 8, 9, 10: n = A,T,C or G
acctgcnnnn

SEQ ID NO: 26 - DNA, 14 bp, Artificial Sequence
  Synthetic construct
  misc_feature 1, 2, 3, 4, 5, 6, 7, 8: n = A,T,C or G
nnnnnnnngcaggt

SEQ ID NO: 27 - DNA, 11 bp, Artificial Sequence
  Synthetic construct
  misc_feature 7, 8, 9, 10, 11: n = A,T,C or G
actgggnnnnn

SEQ ID NO: 28 - DNA, 10 bp, Artificial Sequence
  Synthetic construct
  misc_feature 1, 2, 3, 4: n = A,T,C or G
nnnncccagt

SEQ ID NO: 29 - DNA, 22 bp, Artificial Sequence
  Synthetic construct
  misc_feature 7, 8, 9, 10, 11, 12, 13, 14, 15, 16, 17, 18, 19, 20, 21,22: n = A,T,C or G
ctggagnnnnnnnnnnnnnnnn

SEQ ID NO: 30 - DNA, 20 bp, Artificial Sequence
  Synthetic construct
  misc_feature 1, 2, 3, 4, 5, 6, 7, 8, 9, 10, 11, 12, 13, 14: n = A,T,C or G
nnnnnnnnnnnnnnctccag

SEQ ID NO: 31 - DNA, 22 bp, Artificial Sequence
  Synthetic construct
  misc_feature 7, 8, 9, 10, 11, 12, 13, 14, 15, 16, 17, 18, 19, 20, 21,22: n = A,T,C or G
cttgagnnnnnnnnnnnnnnnn

SEQ ID NO: 32 - DNA, 20 bp, Artificial Sequence
  Synthetic construct
  misc_feature 1, 2, 3, 4, 5, 6, 7, 8, 9, 10, 11, 12, 13, 14: n = A,T,C or G
nnnnnnnnnnnnnnctcaag

SEQ ID NO: 33 - DNA, 7 bp, Artificial Sequence
  Synthetic construct
  misc_feature 7: n = A,T,C or G
ggtctcn

SEQ ID NO: 34 - DNA, 11 bp, Artificial Sequence
  Synthetic construct
  misc_feature 1, 2, 3, 4, 5: n = A,T,C or G
nnnnngagacc

SEQ ID NO: 35 - DNA, 7 bp, Artificial Sequence
  Synthetic construct
  misc_feature 7: n = A,T,C or G
ggtctcn

SEQ ID NO: 36 - DNA, 11 bp, Artificial Sequence
  Synthetic construct
  misc_feature 1, 2, 3, 4, 5: n = A,T,C or G
nnnnngagacc

SEQ ID NO: 37 - DNA, 30 bp, Artificial Sequence
  Synthetic construct
  misc_feature 1, 2, 3, 4, 5, 6, 7, 8, 9, 12, 13, 14, 15, 16, 21, 22,23, 24, 25, 26, 27, 28, 29, 30: n = A,T,C or G
nnnnnnnnnacnnnnnctccnnnnnnnnnn

SEQ ID NO: 38 - DNA, 30 bp, Artificial Sequence
  Synthetic construct
  misc_feature 1, 2, 3, 4, 5, 6, 7, 12, 13, 14, 15, 16, 19, 20, 21, 22,23, 24, 25, 26, 27, 28, 29, 30: n = A,T,C or G
nnnnnnnggagnnnnngtnnnnnnnnnnnn

SEQ ID NO: 39 - DNA, 16 bp, Artificial Sequence
  Synthetic construct
  misc_feature 7, 8, 9, 10, 11, 12, 13, 14, 15, 16: n = A,T,C or G
gaggagnnnnnnnnnn

SEQ ID NO: 40 - DNA, 14 bp, Artificial Sequence
  Synthetic construct
  misc_feature 1, 2, 3, 4, 5, 6, 7, 8: n = A,T,C or G
nnnnnnnnctcctc

SEQ ID NO: 41 - DNA, 22 bp, Artificial Sequence
  Synthetic construct
  misc_feature 7, 8, 9, 10, 11, 12, 13, 14, 15, 16, 17, 18, 19, 20, 21,22: n = A,T,C or G
gtgcagnnnnnnnnnnnnnnnn

SEQ ID NO: 42 - DNA, 20 bp, Artificial Sequence
  Synthetic construct
  misc_feature 1, 2, 3, 4, 5, 6, 7, 8, 9, 10, 11, 12, 13, 14: n = A,T,C or G
nnnnnnnnnnnnnnctgcac

SEQ ID NO: 43 - DNA, 6 bp, Artificial Sequence
  Synthetic construct
  misc_feature 6: n = A,T,C or G
gtctcn

SEQ ID NO: 44 - DNA, 10 bp, Artificial Sequence
  Synthetic construct
  misc_feature 1, 2, 3, 4, 5: n = A,T,C or G
nnnnngagac

SEQ ID NO: 45 - DNA, 7 bp, Artificial Sequence
  Synthetic construct
  misc_feature 7: n = A,T,C or G
cgtctcn

SEQ ID NO: 46 - DNA, 11 bp, Artificial Sequence
  Synthetic construct
  misc_feature 1, 2, 3, 4, 5: n = A,T,C or G
nnnnngagacg

SEQ ID NO: 47 - DNA, 7 bp, Artificial Sequence
  Synthetic construct
  misc_feature 7: n = A,T,C or G
cgtctcn

SEQ ID NO: 48 - DNA, 11 bp, Artificial Sequence
  Synthetic construct
  misc_feature 1, 2, 3, 4, 5: n = A,T,C or G
nnnnngagacg

SEQ ID NO: 49 - DNA, 15 bp, Artificial Sequence
  Synthetic construct
  misc_feature 6, 7, 8, 9, 10, 11, 12, 13, 14, 15: n = A,T,C or G
gggacnnnnnnnnnn

SEQ ID NO: 50 - DNA, 19 bp, Artificial Sequence
  Synthetic construct
  misc_feature 1, 2, 3, 4, 5, 6, 7, 8, 9, 10, 11, 12, 13, 14: n = A,T,C or G
nnnnnnnnnnnnnngtccc

SEQ ID NO: 51 - DNA, 7 bp, Artificial Sequence
  Synthetic construct
  misc_feature 7: n = A,T,C or G
gaatgcn

SEQ ID NO: 52 - DNA, 7 bp, Artificial Sequence
  Synthetic construct
  misc_feature 1: n = A,T,C or G
ngcattc

SEQ ID NO: 53 - DNA, 14 bp, Artificial Sequence
  Synthetic construct
  misc_feature 6, 7, 8, 9, 10, 11, 12, 13, 14: n = A,T,C or G
ctcagnnnnnnnnn

SEQ ID NO: 54 - DNA, 12 bp, Artificial Sequence
  Synthetic construct
  misc_feature 1, 2, 3, 4, 5, 6, 7: n = A,T,C or G
nnnnnnnctgag

SEQ ID NO: 55 - DNA, 10 bp, Artificial Sequence
  Synthetic construct
  misc_feature 7, 8, 9, 10: n = A,T,C or G
acctgcnnnn

SEQ ID NO: 56 - DNA, 13 bp, Artificial Sequence
  Synthetic construct
  misc_feature 1, 2, 3, 4, 5, 6, 7: n = A,T,C or G
nnnnnnngcaggt

SEQ ID NO: 57 - DNA, 8 bp, Artificial Sequence
  Synthetic construct
  misc_feature 8: n = A,T,C or G
gctcttcn

SEQ ID NO: 58 - DNA, 11 bp, Artificial Sequence
  Synthetic construct
  misc_feature 1, 2, 3, 4: n = A,T,C or G
nnnngaagagc

SEQ ID NO: 59 - DNA, 8 bp, Artificial Sequence
  Synthetic construct
  misc_feature 7, 8: n = A,T,C or G
gcaatgnn

SEQ ID NO: 60 - DNA, 6 bp, Artificial Sequence
  Synthetic construct
cattgc

SEQ ID NO: 61 - DNA, 6 bp, Artificial Sequence
  Synthetic construct
  misc_feature 6: n = A,T,C or G
actggn

SEQ ID NO: 62 - DNA, 6 bp, Artificial Sequence
  Synthetic construct
  misc_feature 1: n = A,T,C or G
nccagt

SEQ ID NO: 63 - DNA, 7 bp, Artificial Sequence
  Synthetic construct
  misc_feature 6, 7: n = A,T,C or G
ggatgnn

SEQ ID NO: 64 - DNA, 5 bp, Artificial Sequence
  Synthetic construct
catcc

SEQ ID NO: 65 - DNA, 16 bp, Artificial Sequence
  Synthetic construct
  misc_feature 7, 8, 9, 10, 11, 12, 13, 14, 15, 16: n = A,T,C or G
gcgatgnnnnnnnnnn

SEQ ID NO: 66 - DNA, 20 bp, Artificial Sequence
  Synthetic construct
  misc_feature 1, 2, 3, 4, 5, 6, 7, 8, 9, 10, 11, 12, 13, 14: n = A,T,C or G
nnnnnnnnnnnnnncatcgc

SEQ ID NO: 67 - DNA, 8 bp, Artificial Sequence
  Synthetic construct
  misc_feature 7, 8: n = A,T,C or G
gcagtgnn

SEQ ID NO: 68 - DNA, 6 bp, Artificial Sequence
  Synthetic construct
cactgc

SEQ ID NO: 69 - DNA, 7 bp, Artificial Sequence
  Synthetic construct
  misc_feature 6, 7: n = A,T,C or G
ggatgnn

SEQ ID NO: 70 - DNA, 5 bp, Artificial Sequence
  Synthetic construct
catcc

SEQ ID NO: 71 - DNA, 8 bp, Artificial Sequence
  Synthetic construct
  misc_feature 7, 8: n = A,T,C or G
gcagtgnn

SEQ ID NO: 72 - DNA, 6 bp, Artificial Sequence
  Synthetic construct
cactgc

SEQ ID NO: 73 - DNA, 7 bp, Artificial Sequence
  Synthetic construct
  misc_feature 6, 7: n = A,T,C or G
cagtgnn

SEQ ID NO: 74 - DNA, 5 bp, Artificial Sequence
  Synthetic construct
cactg

SEQ ID NO: 75 - DNA, 35 bp, Artificial Sequence
  Synthetic construct
  misc_feature 1, 2, 3, 4, 5, 6, 7, 8, 9, 10, 11, 15, 16, 17, 18, 19,24, 25, 26, 27, 28, 29, 30, 31, 32, 33, 34, 35: n = A,T,C or G
nnnnnnnnnnncaannnnngtggnnnnnnnnnnnn

SEQ ID NO: 76 - DNA, 35 bp, Artificial Sequence
  Synthetic construct
  misc_feature 1, 2, 3, 4, 5, 6, 7, 8, 9, 10, 15, 16, 17, 18, 19, 23,24, 25, 26, 27, 28, 29, 30, 31, 32, 33, 34, 35: n = A,T,C or G
nnnnnnnnnnccacnnnnnttgnnnnnnnnnnnnn

SEQ ID NO: 77 - DNA, 7 bp, Artificial Sequence
  Synthetic construct
  misc_feature 7: n = A,T,C or G
ctcttcn

SEQ ID NO: 78 - DNA, 10 bp, Artificial Sequence
  Synthetic construct
  misc_feature 1, 2, 3, 4: n = A,T,C or G
nnnngaagag

SEQ ID NO: 79 - DNA, 17 bp, Artificial Sequence
  Synthetic construct
  misc_feature 7, 8, 9, 10, 11, 12, 13, 14, 15, 16, 17: n = A,T,C or G
ggcggannnnnnnnnnn

SEQ ID NO: 80 - DNA, 15 bp, Artificial Sequence
  Synthetic construct
  misc_feature 1, 2, 3, 4, 5, 6, 7, 8, 9: n = A,T,C or G
nnnnnnnnntccgcc

SEQ ID NO: 81 - DNA, 7 bp, Artificial Sequence
  Synthetic construct
  misc_feature 7: n = A,T,C or G
cgtctcn

SEQ ID NO: 82 - DNA, 11 bp, Artificial Sequence
  Synthetic construct
  misc_feature 1, 2, 3, 4, 5: n = A,T,C or G
nnnnngagacg

SEQ ID NO: 83 - DNA, 9 bp, Artificial Sequence
  Synthetic construct
  misc_feature 6, 7, 8, 9: n = A,T,C or G
cccgcnnnn

SEQ ID NO: 84 - DNA, 11 bp, Artificial Sequence
  Synthetic construct
  misc_feature 1, 2, 3, 4, 5, 6: n = A,T,C or G
nnnnnngcggg

SEQ ID NO: 85 - DNA, 14 bp, Artificial Sequence
  Synthetic construct
  misc_feature 6, 7, 8, 9, 10, 11, 12, 13, 14: n = A,T,C or G
ggatgnnnnnnnnn

SEQ ID NO: 86 - DNA, 18 bp, Artificial Sequence
  Synthetic construct
  misc_feature 1, 2, 3, 4, 5, 6, 7, 8, 9, 10, 11, 12, 13: n = A,T,C or G
nnnnnnnnnnnnncatcc

SEQ ID NO: 87 - DNA, 10 bp, Artificial Sequence
  Synthetic construct
  misc_feature 6, 7, 8, 9, 10: n = A,T,C or G
gacgcnnnnn

SEQ ID NO: 88 - DNA, 15 bp, Artificial Sequence
  Synthetic construct
  misc_feature 1, 2, 3, 4, 5, 6, 7, 8, 9, 10: n = A,T,C or G
nnnnnnnnnngcgtc

SEQ ID NO: 89 - DNA, 13 bp, Artificial Sequence
  Synthetic construct
  misc_feature 6, 7, 8, 9, 10, 11, 12, 13: n = A,T,C or G
ggtgannnnnnnn

SEQ ID NO: 90 - DNA, 12 bp, Artificial Sequence
  Synthetic construct
  misc_feature 1, 2, 3, 4, 5, 6, 7: n = A,T,C or G
nnnnnnntcacc

SEQ ID NO: 91 - DNA, 11 bp, Artificial Sequence
  Synthetic construct
  misc_feature 6, 7, 8, 9, 10, 11: n = A,T,C or G
ccttcnnnnnn

SEQ ID NO: 92 - DNA, 10 bp, Artificial Sequence
  Synthetic construct
  misc_feature 1, 2, 3, 4, 5: n = A,T,C or G
nnnnngaagg

SEQ ID NO: 93 - DNA, 13 bp, Artificial Sequence
  Synthetic construct
  misc_feature 6, 7, 8, 9, 10, 11, 12, 13: n = A,T,C or G
gaagannnnnnnn

SEQ ID NO: 94 - DNA, 12 bp, Artificial Sequence
  Synthetic construct
  misc_feature 1, 2, 3, 4, 5, 6, 7: n = A,T,C or G
nnnnnnntcttc

SEQ ID NO: 95 - DNA, 10 bp, Artificial Sequence
  Synthetic construct
  misc_feature 6, 7, 8, 9, 10: n = A,T,C or G
gagtcnnnnn

SEQ ID NO: 96 - DNA, 10 bp, Artificial Sequence
  Synthetic construct
  misc_feature 1, 2, 3, 4, 5: n = A,T,C or G
nnnnngactc

SEQ ID NO: 97 - DNA, 26 bp, Artificial Sequence
  Synthetic construct
  misc_feature 7, 8, 9, 10, 11, 12, 13, 14, 15, 16, 17, 18, 19, 20, 21,22, 23, 24, 25, 26: n = A,T,C or G
tccracnnnnnnnnnnnnnnnnnnnn

SEQ ID NO: 98 - DNA, 24 bp, Artificial Sequence
  Synthetic construct
  misc_feature 1, 2, 3, 4, 5, 6, 7, 8, 9, 10, 11, 12, 13, 14, 15, 16,17, 18: n = A,T,C or G
nnnnnnnnnnnnnnnnnngtygga

SEQ ID NO: 99 - DNA, 11 bp, Artificial Sequence
  Synthetic construct
  misc_feature 5, 6, 7, 8, 9, 10, 11: n = A,T,C or G
cctcnnnnnnn

SEQ ID NO: 100 - DNA, 10 bp, Artificial Sequence
  Synthetic construct
  misc_feature 1, 2, 3, 4, 5, 6: n = A,T,C or G
nnnnnngagg

SEQ ID NO: 101 - DNA, 27 bp, Artificial Sequence
  Synthetic construct
  misc_feature 7, 8, 9, 10, 11, 12, 13, 14, 15, 16, 17, 18, 19, 20, 21,22, 23, 24, 25, 26, 27: n = A,T,C or G
gccgagnnnnnnnnnnnnnnnnnnnnn

SEQ ID NO: 102 - DNA, 25 bp, Artificial Sequence
  Synthetic construct
  misc_feature 1, 2, 3, 4, 5, 6, 7, 8, 9, 10, 11, 12, 13, 14, 15, 16,17, 18, 19: n = A,T,C or G
nnnnnnnnnnnnnnnnnnnctcggc

SEQ ID NO: 103 - DNA, 9 bp, Artificial Sequence
  Synthetic construct
  misc_feature 6, 7, 8, 9: n = A,T,C or G
gagtcnnnn

SEQ ID NO: 104 - DNA, 10 bp, Artificial Sequence
  Synthetic construct
  misc_feature 1, 2, 3, 4, 5: n = A,T,C or G
nnnnngactc

SEQ ID NO: 105 - DNA, 8 bp, Artificial Sequence
  Synthetic construct
  misc_feature 8: n = A,T,C or G
gctcttcn

SEQ ID NO: 106 - DNA, 11 bp, Artificial Sequence
  Synthetic construct
  misc_feature 1, 2, 3, 4: n = A,T,C or G
nnnngaagagc

SEQ ID NO: 107 - DNA, 10 bp, Artificial Sequence
  Synthetic construct
  misc_feature 6, 7, 8, 9, 10: n = A,T,C or G
gcatcnnnnn

SEQ ID NO: 108 - DNA, 14 bp, Artificial Sequence
  Synthetic construct
  misc_feature 1, 2, 3, 4, 5, 6, 7, 8, 9: n = A,T,C or G
nnnnnnnnngatgc